SEQUENCE LISTING (430 sequences)

SEQ ID NO: 1 - protein, 16 aa, synthetic construct
  REGION 1..16: note = Synthetic Peptide
RADARADARADARADA

SEQ ID NO: 2 - protein, 16 aa, synthetic construct
  REGION 1..16: note = Synthetic Peptide
SGSGSGSGSGSGSGSG

SEQ ID NO: 3 - protein, 16 aa, synthetic construct
  REGION 1..16: note = Synthetic Peptide
SASASASASASASASA

SEQ ID NO: 4 - protein, 16 aa, synthetic construct
  REGION 1..16: note = Synthetic Peptide
SVSVSVSVSVSVSVSV

SEQ ID NO: 5 - protein, 16 aa, synthetic construct
  REGION 1..16: note = Synthetic Peptide
SLSLSLSLSLSLSLSL

SEQ ID NO: 6 - protein, 16 aa, synthetic construct
  REGION 1..16: note = Synthetic Peptide
SISISISISISISISI

SEQ ID NO: 7 - protein, 16 aa, synthetic construct
  REGION 1..16: note = Synthetic Peptide
SMSMSMSMSMSMSMSM

SEQ ID NO: 8 - protein, 16 aa, synthetic construct
  REGION 1..16: note = Synthetic Peptide
SFSFSFSFSFSFSFSF

SEQ ID NO: 9 - protein, 16 aa, synthetic construct
  REGION 1..16: note = Synthetic Peptide
SWSWSWSWSWSWSWSW

SEQ ID NO: 10 - protein, 16 aa, synthetic construct
  REGION 1..16: note = Synthetic Peptide
SPSPSPSPSPSPSPSP

SEQ ID NO: 11 - protein, 16 aa, synthetic construct
  REGION 1..16: note = Synthetic Peptide
TGTGTGTGTGTGTGTG

SEQ ID NO: 12 - protein, 16 aa, synthetic construct
  REGION 1..16: note = Synthetic Peptide
TATATATATATATATA

SEQ ID NO: 13 - protein, 16 aa, synthetic construct
  REGION 1..16: note = Synthetic Peptide
TVTVTVTVTVTVTVTV

SEQ ID NO: 14 - protein, 16 aa, synthetic construct
  REGION 1..16: note = Synthetic Peptide
TLTLTLTLTLTLTLTL

SEQ ID NO: 15 - protein, 16 aa, synthetic construct
  REGION 1..16: note = Synthetic Peptide
TITITITITITITITI

SEQ ID NO: 16 - protein, 16 aa, synthetic construct
  REGION 1..16: note = Synthetic Peptide
TMTMTMTMTMTMTMTM

SEQ ID NO: 17 - protein, 16 aa, synthetic construct
  REGION 1..16: note = Synthetic Peptide
TFTFTFTFTFTFTFTF

SEQ ID NO: 18 - protein, 16 aa, synthetic construct
  REGION 1..16: note = Synthetic Peptide
TWTWTWTWTWTWTWTW

SEQ ID NO: 19 - protein, 16 aa, synthetic construct
  REGION 1..16: note = Synthetic Peptide
TPTPTPTPTPTPTPTP

SEQ ID NO: 20 - protein, 16 aa, synthetic construct
  REGION 1..16: note = Synthetic Peptide
CGCGCGCGCGCGCGCG

SEQ ID NO: 21 - protein, 16 aa, synthetic construct
  REGION 1..16: note = Synthetic Peptide
CACACACACACACACA

SEQ ID NO: 22 - protein, 16 aa, synthetic construct
  REGION 1..16: note = Synthetic Peptide
CVCVCVCVCVCVCVCV

SEQ ID NO: 23 - protein, 16 aa, synthetic construct
  REGION 1..16: note = Synthetic Peptide
CLCLCLCLCLCLCLCL

SEQ ID NO: 24 - protein, 16 aa, synthetic construct
  REGION 1..16: note = Synthetic Peptide
CICICICICICICICI

SEQ ID NO: 25 - protein, 16 aa, synthetic construct
  REGION 1..16: note = Synthetic Peptide
CMCMCMCMCMCMCMCM

SEQ ID NO: 26 - protein, 16 aa, synthetic construct
  REGION 1..16: note = Synthetic Peptide
CFCFCFCFCFCFCFCF

SEQ ID NO: 27 - protein, 15 aa, synthetic construct
  REGION 1..15: note = Synthetic Peptide
CWCWCWCWCWCWCWC

SEQ ID NO: 28 - protein, 16 aa, synthetic construct
  REGION 1..16: note = Synthetic Peptide
CPCPCPCPCPCPCPCP

SEQ ID NO: 29 - protein, 16 aa, synthetic construct
  REGION 1..16: note = Synthetic Peptide
YGYGYGYGYGYGYGYG

SEQ ID NO: 30 - protein, 16 aa, synthetic construct
  REGION 1..16: note = Synthetic Peptide
YAYAYAYAYAYAYAYA

SEQ ID NO: 31 - protein, 16 aa, synthetic construct
  REGION 1..16: note = Synthetic Peptide
YVYVYVYVYVYVYVYV

SEQ ID NO: 32 - protein, 16 aa, synthetic construct
  REGION 1..16: note = Synthetic Peptide
YLYLYLYLYLYLYLYL

SEQ ID NO: 33 - protein, 16 aa, synthetic construct
  REGION 1..16: note = Synthetic Peptide
YIYIYIYIYIYIYIYI

SEQ ID NO: 34 - protein, 16 aa, synthetic construct
  REGION 1..16: note = Synthetic Peptide
YMYMYMYMYMYMYMYM

SEQ ID NO: 35 - protein, 16 aa, synthetic construct
  REGION 1..16: note = Synthetic Peptide
YFYFYFYFYFYFYFYF

SEQ ID NO: 36 - protein, 16 aa, synthetic construct
  REGION 1..16: note = Synthetic Peptide
YWYWYWYWYWYWYWYW

SEQ ID NO: 37 - protein, 16 aa, synthetic construct
  REGION 1..16: note = Synthetic Peptide
YPYPYPYPYPYPYPYP

SEQ ID NO: 38 - protein, 16 aa, synthetic construct
  REGION 1..16: note = Synthetic Peptide
NGNGNGNGNGNGNGNG

SEQ ID NO: 39 - protein, 16 aa, synthetic construct
  REGION 1..16: note = Synthetic Peptide
NANANANANANANANA

SEQ ID NO: 40 - protein, 16 aa, synthetic construct
  REGION 1..16: note = Synthetic Peptide
NVNVNVNVNVNVNVNV

SEQ ID NO: 41 - protein, 16 aa, synthetic construct
  REGION 1..16: note = Synthetic Peptide
NLNLNLNLNLNLNLNL

SEQ ID NO: 42 - protein, 16 aa, synthetic construct
  REGION 1..16: note = Synthetic Peptide
NINININININININI

SEQ ID NO: 43 - protein, 16 aa, synthetic construct
  REGION 1..16: note = Synthetic Peptide
NMNMNMNMNMNMNMNM

SEQ ID NO: 44 - protein, 16 aa, synthetic construct
  REGION 1..16: note = Synthetic Peptide
NFNFNFNFNFNFNFNF

SEQ ID NO: 45 - protein, 16 aa, synthetic construct
  REGION 1..16: note = Synthetic Peptide
NWNWNWNWNWNWNWNW

SEQ ID NO: 46 - protein, 16 aa, synthetic construct
  REGION 1..16: note = Synthetic Peptide
NPNPNPNPNPNPNPNP

SEQ ID NO: 47 - protein, 16 aa, synthetic construct
  REGION 1..16: note = Synthetic Peptide
QGQGQGQGQGQGQGQG

SEQ ID NO: 48 - protein, 16 aa, synthetic construct
  REGION 1..16: note = Synthetic Peptide
QAQAQAQAQAQAQAQA

SEQ ID NO: 49 - protein, 16 aa, synthetic construct
  REGION 1..16: note = Synthetic Peptide
QVQVQVQVQVQVQVQV

SEQ ID NO: 50 - protein, 16 aa, synthetic construct
  REGION 1..16: note = Synthetic Peptide
QLQLQLQLQLQLQLQL

SEQ ID NO: 51 - protein, 16 aa, synthetic construct
  REGION 1..16: note = Synthetic Peptide
QIQIQIQIQIQIQIQI

SEQ ID NO: 52 - protein, 16 aa, synthetic construct
  REGION 1..16: note = Synthetic Peptide
QMQMQMQMQMQMQMQM

SEQ ID NO: 53 - protein, 16 aa, synthetic construct
  REGION 1..16: note = Synthetic Peptide
QFQFQFQFQFQFQFQF

SEQ ID NO: 54 - protein, 16 aa, synthetic construct
  REGION 1..16: note = Synthetic Peptide
QWQWQWQWQWQWQWQW

SEQ ID NO: 55 - protein, 16 aa, synthetic construct
  REGION 1..16: note = Synthetic Peptide
QPQPQPQPQPQPQPQP

SEQ ID NO: 56 - protein, 16 aa, synthetic construct
  REGION 1..16: note = Synthetic Peptide
AEAKAEAKAEAKAEAK

SEQ ID NO: 57 - protein, 4 aa, synthetic construct
  REGION 1..4: note = Synthetic Peptide
RADA

SEQ ID NO: 58 - protein, 16 aa, synthetic construct
  REGION 1..16: note = Synthetic Peptide
RAEARAEARAEARAEA

SEQ ID NO: 59 - protein, 16 aa, synthetic construct
  REGION 1..16: note = Synthetic Peptide
KADAKADAKADAKADA

SEQ ID NO: 60 - protein, 13 aa, synthetic construct
  REGION 1..13: note = Synthetic Peptide
ARADARADARADA

SEQ ID NO: 61 - protein, 20 aa, synthetic construct
  REGION 1..20: note = Synthetic Peptide
RADARADARADARADARADA

SEQ ID NO: 62 - protein, 21 aa, synthetic construct
  REGION 1..21: note = Synthetic Peptide
ARADARADARADARADARADA

SEQ ID NO: 63 - protein, 17 aa, synthetic construct
  REGION 1..17: note = Synthetic Peptide
ARADARADARADARADA

SEQ ID NO: 64 - protein, 16 aa, synthetic construct
  REGION 1..16: note = Synthetic Peptide
RLDLRLDLRLDLRLDL

SEQ ID NO: 65 - protein, 4 aa, synthetic construct
  REGION 1..4: note = Synthetic Peptide
RLDL

SEQ ID NO: 66 - protein, 6 aa, synthetic construct
  REGION 1..6: note = Synthetic Peptide
RLDLRL

SEQ ID NO: 67 - protein, 6 aa, synthetic construct
  REGION 1..6: note = Synthetic Peptide
RADARA

SEQ ID NO: 68 - protein, 6 aa, synthetic construct
  REGION 1..6: note = Synthetic Peptide
LRLDLR

SEQ ID NO: 69 - protein, 13 aa, synthetic construct
  REGION 1..13: note = Synthetic Peptide
IEIKIEIKIEIKI

SEQ ID NO: 70 - protein, 16 aa, synthetic construct
  REGION 1..16: note = Synthetic Peptide
IEIKIEIKIEIKIEIK

SEQ ID NO: 71 - protein, 17 aa, synthetic construct
  REGION 1..17: note = Synthetic Peptide
IEIKIEIKIEIKIEIKI

SEQ ID NO: 72 - protein, 20 aa, synthetic construct
  REGION 1..20: note = Synthetic Peptide
IEIKIEIKIEIKIEIKIEIK

SEQ ID NO: 73 - protein, 21 aa, synthetic construct
  REGION 1..21: note = Synthetic Peptide
IEIKIEIKIEIKIEIKIEIKI

SEQ ID NO: 74 - protein, 12 aa, synthetic construct
  REGION 1..12: note = Synthetic Peptide
IEIKIEIKIEIK

SEQ ID NO: 75 - protein, 16 aa, synthetic construct
  REGION 1..16: note = Synthetic Peptide
EIKIEIKIEIKIEIKI

SEQ ID NO: 76 - protein, 16 aa, synthetic construct
  REGION 1..16: note = Synthetic Peptide
EAKAEAKAEAKAEAKA

SEQ ID NO: 77 - protein, 4 aa, synthetic construct
  REGION 1..4: note = Synthetic Peptide
EAKA

SEQ ID NO: 78 - protein, 10 aa, synthetic construct
  REGION 1..10: note = Synthetic Peptide
EAKAEAKAEA

SEQ ID NO: 79 - protein, 20 aa, synthetic construct
  REGION 1..20: note = Synthetic Peptide
EAKAEAKAEAKAEAKAEAKA

SEQ ID NO: 80 - protein, 17 aa, synthetic construct
  REGION 1..17: note = Synthetic Peptide
AEAKAEAKAEAKAEAKA

SEQ ID NO: 81 - protein, 13 aa, synthetic construct
  REGION 1..13: note = Synthetic Peptide
AEAKAEAKAEAKA

SEQ ID NO: 82 - protein, 18 aa, synthetic construct
  REGION 1..18: note = Synthetic Peptide
RADARADARADARADLRA

SEQ ID NO: 83 - protein, 16 aa, synthetic construct
  REGION 1..16: note = Synthetic Peptide
  SITE 1: note = D-arginine
  SITE 2: note = D-alanine
  SITE 3: note = D-aspartic acid
  SITE 4: note = D-alanine
  SITE 5: note = D-arginine
  SITE 6: note = D-alanine
  SITE 7: note = D-aspartic acid
  SITE 8: note = D-alanine
  SITE 9: note = D-arginine
  SITE 10: note = D-alanine
  SITE 11: note = D-aspartic acid
  SITE 12: note = D-alanine
  SITE 13: note = D-arginine
  SITE 14: note = D-alanine
  SITE 15: note = D-aspartic acid
  SITE 16: note = D-alanine
RADARADARADARADA

SEQ ID NO: 84 - protein, 12 aa, synthetic construct
  REGION 1..12: note = Synthetic Peptide
  SITE 1: note = D-arginine
  SITE 2: note = D-alanine
  SITE 3: note = D-aspartic acid
  SITE 4: note = D-alanine
  SITE 5: note = D-arginine
  SITE 6: note = D-alanine
  SITE 7: note = D-aspartic acid
  SITE 8: note = D-alanine
  SITE 9: note = D-arginine
  SITE 10: note = D-alanine
  SITE 11: note = D-aspartic acid
  SITE 12: note = D-alanine
RADARADARADA

SEQ ID NO: 85 - protein, 12 aa, synthetic construct
  REGION 1..12: note = Synthetic Peptide
  SITE 1: note = D-glutamic acid
  SITE 2: note = D-alanine
  SITE 3: note = D-lysine
  SITE 4: note = D-alanine
  SITE 5: note = D-glutamic acid
  SITE 6: note = D-alanine
  SITE 7: note = D-lysine
  SITE 8: note = D-alanine
  SITE 9: note = D-glutamic acid
  SITE 10: note = D-alanine
  SITE 11: note = D-lysine
  SITE 12: note = D-alanine
EAKAEAKAEAKA

SEQ ID NO: 86 - protein, 4 aa, synthetic construct
  REGION 1..4: note = Synthetic Peptide
  SITE 1: note = D-glutamic acid
  SITE 2: note = D-alanine
  SITE 3: note = D-lysine
  SITE 4: note = D-alanine
EAKA

SEQ ID NO: 87 - protein, 4 aa, synthetic construct
  REGION 1..4: note = Synthetic Peptide
  SITE 1: note = D-arginine
  SITE 2: note = D-alanine
  SITE 3: note = D-aspartic acid
  SITE 4: note = D-alanine
RADA

SEQ ID NO: 88 - protein, 8 aa, synthetic construct
  REGION 1..8: note = Synthetic Peptide
RADARADA

SEQ ID NO: 89 - protein, 16 aa, synthetic construct
  REGION 1..16: note = Synthetic Peptide
EARAEARAEARAEARA

SEQ ID NO: 90 - protein, 12 aa, synthetic construct
  REGION 1..12: note = Synthetic Peptide
EARAEARAEARA

SEQ ID NO: 91 - protein, 9 aa, synthetic construct
  REGION 1..9: note = Synthetic Peptide
EARAEARAE

SEQ ID NO: 92 - protein, 4 aa, synthetic construct
  REGION 1..4: note = Synthetic Peptide
EARA

SEQ ID NO: 93 - protein, 9 aa, synthetic construct
  REGION 1..9: note = Synthetic Peptide
  SITE 9: note = C-terminal amine (NH2)
CYIQNCPRG

SEQ ID NO: 94 - protein, 7 aa, synthetic construct
  REGION 1..7: note = Synthetic Peptide
  REGION 4..5: note = amino succinyl modification
  SITE 7: note = C-terminal amine (NH2)
YFQNPRG

SEQ ID NO: 95 - protein, 7 aa, synthetic construct
  REGION 1..7: note = Synthetic Peptide
  REGION 4..5: note = amino succinyl modification
  SITE 7: note = C-terminal amine (NH2)
YIQNPRG

SEQ ID NO: 96 - protein, 7 aa, synthetic construct
  SITE 1: note = MISC_FEATURE - 3-mercaptopropionyl-D-Pyridylanine
  SITE 7: note = C-terminal amine (NH2)
FQNCPRG

SEQ ID NO: 97 - protein, 10 aa, synthetic construct
  SITE 1: note = X = deamino penicillamine
  SITE 10: note = C-terminal amine (NH2)
XYFVNCPDRG

SEQ ID NO: 98 - protein, 8 aa, synthetic construct
  SITE 1: note = MISC_FEATURE - 3-mercaptopropionyl group
  SITE 8: note = NH2 - C-terminal amine
  REGION 1..8: note = Synthetic Peptide
YFQNCPRG

SEQ ID NO: 99 - protein, 9 aa, synthetic construct
  REGION 1..9: note = Synthetic Peptide
  SITE 1: note = MISC_FEATURE - 3-mercaptopropionyl group
  SITE 9: note = C-terminal amine (NH2)
YFQNCPDRG

SEQ ID NO: 100 - protein, 7 aa, synthetic construct
  REGION 1..7: note = Synthetic Peptide
  SITE 1: note = MISC_FEATURE - 3-mercaptopropionyl group
YFQNCPK

SEQ ID NO: 101 - protein, 9 aa, synthetic construct
  REGION 1..9: note = Synthetic Peptide
  SITE 9: note = C-terminal amine (NH2)
CYFQNCPKG

SEQ ID NO: 102 - protein, 8 aa, synthetic construct
  REGION 1..8: note = Synthetic Peptide
CYFQNCPK

SEQ ID NO: 103 - protein, 9 aa, synthetic construct
  REGION 1..9: note = Synthetic Peptide
  SITE 1: note = MISC_FEATURE - 3-mercaptopropionyl group
  SITE 9: note = C-terminal amine (NH2)
YFVNCPDRG

SEQ ID NO: 104 - protein, 9 aa, synthetic construct
  REGION 1..9: note = Synthetic Peptide
  SITE 8: note = X = ornithine
  SITE 9: note = C-terminal amine (NH2)
CFIQNCPXG

SEQ ID NO: 105 - protein, 9 aa, synthetic construct
  REGION 1..9: note = Synthetic Peptide
  SITE 1: note = MISC_FEATURE - pyridoxamine phosphate group
  SITE 2: note = MISC_FEATURE - ethoxy group
  SITE 8: note = X = citrulline
  SITE 9: note = C-terminal amine (NH2)
DYFVNCPXG

SEQ ID NO: 106 - protein, 8 aa, synthetic construct
  REGION 1..8: note = Synthetic Peptide
  SITE 1: note = MISC_FEATURE - pyridoxamine phosphate group
  SITE 1: note = MISC_FEATURE - ethoxy (OET) group
  SITE 8: note = C-terminal amine (NH2)
YFVNCPRG

SEQ ID NO: 107 - protein, 8 aa, synthetic construct
  REGION 1..8: note = Synthetic Peptide
  SITE 1: note = MISC_FEATURE - pyridoxamine phosphate modification
  MOD_RES 1: note = Methylated tyrosine
  SITE 8: note = C-terminal amine (NH2)
YFQNCPRG

SEQ ID NO: 108 - protein, 8 aa, synthetic construct
  REGION 1..8: note = Synthetic Peptide
  SITE 1: note = MISC_FEATURE - pyridoxamine phosphate group
  MOD_RES 1: note = Methylation
  SITE 7: note = X = ornithine
  SITE 8: note = C-terminal amine (NH2)
YIQNCPXG

SEQ ID NO: 109 - protein, 7 aa, synthetic construct
  REGION 1..7: note = Synthetic Peptide
GDRGDSP

SEQ ID NO: 110 - protein, 11 aa, synthetic construct
  REGION 1..11: note = Synthetic Peptide
GDRGDSPASSK

SEQ ID NO: 111 - protein, 10 aa, synthetic construct
  REGION 1..10: note = Synthetic Peptide
  SITE 2: note = X = penicillamine
GXGRGDSPCA

SEQ ID NO: 112 - protein, 6 aa, synthetic construct
  REGION 1..6: note = Synthetic Peptide
GRADSP

SEQ ID NO: 113 - protein, 7 aa, synthetic construct
  REGION 1..7: note = Synthetic Peptide
GRGDDSP

SEQ ID NO: 114 - protein, 6 aa, synthetic construct
  REGION 1..6: note = Synthetic Peptide
GRGDNP

SEQ ID NO: 115 - protein, 5 aa, synthetic construct
  REGION 1..5: note = Synthetic Peptide
GRGDS

SEQ ID NO: 116 - protein, 6 aa, synthetic construct
  REGION 1..6: note = Synthetic Peptide
GRGDSP

SEQ ID NO: 117 - protein, 7 aa, synthetic construct
  REGION 1..7: note = Synthetic Peptide
GRGDSPC

SEQ ID NO: 118 - protein, 7 aa, synthetic construct
  REGION 1..7: note = Synthetic Peptide
GRGDSPK

SEQ ID NO: 119 - protein, 6 aa, synthetic construct
  REGION 1..6: note = Synthetic Peptide
GRGDTP

SEQ ID NO: 120 - protein, 5 aa, synthetic construct
  REGION 1..5: note = Synthetic Peptide
GRGES

SEQ ID NO: 121 - protein, 6 aa, synthetic construct
  REGION 1..6: note = Synthetic Peptide
GRGESP

SEQ ID NO: 122 - protein, 6 aa, synthetic construct
  REGION 1..6: note = Synthetic Peptide
GRGETP

SEQ ID NO: 123 - protein, 4 aa, synthetic construct
  REGION 1..4: note = Synthetic Peptide
KGDS

SEQ ID NO: 124 - protein, 6 aa, synthetic construct
  REGION 1..6: note = Synthetic Peptide
GAVSTA

SEQ ID NO: 125 - protein, 6 aa, synthetic construct
  REGION 1..6: note = Synthetic Peptide
WTVPTA

SEQ ID NO: 126 - protein, 11 aa, synthetic construct
  REGION 1..11: note = Synthetic Peptide
TDVNGDGRHDL

SEQ ID NO: 127 - protein, 4 aa, synthetic construct
  REGION 1..4: note = Synthetic Peptide
REDV

SEQ ID NO: 128 - protein, 4 aa, synthetic construct
  REGION 1..4: note = Synthetic Peptide
RGDC

SEQ ID NO: 129 - protein, 4 aa, synthetic construct
  REGION 1..4: note = Synthetic Peptide
RGDS

SEQ ID NO: 130 - protein, 10 aa, synthetic construct
  REGION 1..10: note = Synthetic Peptide
RGDSPASSKP

SEQ ID NO: 131 - protein, 4 aa, synthetic construct
  REGION 1..4: note = Synthetic Peptide
RGDT

SEQ ID NO: 132 - protein, 4 aa, synthetic construct
  REGION 1..4: note = Synthetic Peptide
RGDV

SEQ ID NO: 133 - protein, 4 aa, synthetic construct
  REGION 1..4: note = Synthetic Peptide
RGES

SEQ ID NO: 134 - protein, 4 aa, synthetic construct
  REGION 1..4: note = Synthetic Peptide
SDGR

SEQ ID NO: 135 - protein, 5 aa, synthetic construct
  REGION 1..5: note = Synthetic Peptide
SDGRG

SEQ ID NO: 136 - protein, 5 aa, synthetic construct
  REGION 1..5: note = Synthetic Peptide
YRGDS

SEQ ID NO: 137 - protein, 14 aa, synthetic construct [sequence omitted]

SEQ ID NO: 138 - protein, 17 aa, synthetic construct
  REGION 1..17: note = Synthetic Peptide
YADSGEGDFLAEGGGVR

SEQ ID NO: 139 - protein, 14 aa, synthetic construct
  REGION 1..14: note = Synthetic Peptide
  SITE 1: note = MISC_FEATURE - Glp modification
GVNDNEEGFFSARY

SEQ ID NO: 140 - protein, 8 aa, synthetic construct
  REGION 1..8: note = Synthetic Peptide
CVSVPQAL

SEQ ID NO: 141 - protein, 7 aa, synthetic construct
  REGION 1..7: note = Synthetic Peptide
MSCRAMM

SEQ ID NO: 142 - protein, 16 aa, synthetic construct
  REGION 1..16: note = Synthetic Peptide
GGGGGDGDGDGDGDGD

SEQ ID NO: 143 - protein, 16 aa, synthetic construct
  REGION 1..16: note = Synthetic Peptide
GGGGGEGEGEGEGEGE

SEQ ID NO: 144 - protein, 16 aa, synthetic construct
  REGION 1..16: note = Synthetic Peptide
GGGGGKGKGKGKGKGK

SEQ ID NO: 145 - protein, 16 aa, synthetic construct
  REGION 1..16: note = Synthetic Peptide
GGGGGRGRGRGRGRGR

SEQ ID NO: 146 - protein, 16 aa, synthetic construct
  REGION 1..16: note = Synthetic Peptide
GGGGGHGHGHGHGHGH

SEQ ID NO: 147 - protein, 16 aa, synthetic construct
  REGION 1..16: note = Synthetic Peptide
AAAAADADADADADAD

SEQ ID NO: 148 - protein, 16 aa, synthetic construct
  REGION 1..16: note = Synthetic Peptide
AAAAAEAEAEAEAEAE

SEQ ID NO: 149 - protein, 16 aa, synthetic construct
  REGION 1..16: note = Synthetic Peptide
AAAAAKAKAKAKAKAK

SEQ ID NO: 150 - protein, 16 aa, synthetic construct
  REGION 1..16: note = Synthetic Peptide
AAAAARARARARARAR

SEQ ID NO: 151 - protein, 16 aa, synthetic construct
  REGION 1..16: note = Synthetic Peptide
AAAAAHAHAHAHAHAH

SEQ ID NO: 152 - protein, 16 aa, synthetic construct
  REGION 1..16: note = Synthetic Peptide
VVVVVDVDVDVDVDVD

SEQ ID NO: 153 - protein, 16 aa, synthetic construct
  REGION 1..16: note = Synthetic Peptide
VVVVVEVEVEVEVEVE

SEQ ID NO: 154 - protein, 16 aa, synthetic construct
  REGION 1..16: note = Synthetic Peptide
VVVVVKVKVKVKVKVK

SEQ ID NO: 155 - protein, 16 aa, synthetic construct
  REGION 1..16: note = Synthetic Peptide
VVVVVRVRVRVRVRVR

SEQ ID NO: 156 - protein, 16 aa, synthetic construct
  REGION 1..16: note = Synthetic Peptide
VVVVVHVHVHVHVHVH

SEQ ID NO: 157 - protein, 16 aa, synthetic construct
  REGION 1..16: note = Synthetic Peptide
LLLLLDLDLDLDLDLD

SEQ ID NO: 158 - protein, 16 aa, synthetic construct
  REGION 1..16: note = Synthetic Peptide
LLLLLELELELELELE

SEQ ID NO: 159 - protein, 16 aa, synthetic construct
  REGION 1..16: note = Synthetic Peptide
LLLLLKLKLKLKLKLK

SEQ ID NO: 160 - protein, 16 aa, synthetic construct
  REGION 1..16: note = Synthetic Peptide
LLLLLRLRLRLRLRLR

SEQ ID NO: 161 - protein, 16 aa, synthetic construct
  REGION 1..16: note = Synthetic Peptide
LLLLLHLHLHLHLHLH

SEQ ID NO: 162 - protein, 16 aa, synthetic construct
  REGION 1..16: note = Synthetic Peptide
IIIIIDIDIDIDIDID

SEQ ID NO: 163 - protein, 16 aa, synthetic construct
  REGION 1..16: note = Synthetic Peptide
IIIIIEIEIEIEIEIE

SEQ ID NO: 164 - protein, 16 aa, synthetic construct
  REGION 1..16: note = Synthetic Peptide
IIIIIKIKIKIKIKIK

SEQ ID NO: 165 - protein, 16 aa, synthetic construct
  REGION 1..16: note = Synthetic Peptide
IIIIIRIRIRIRIRIR

SEQ ID NO: 166 - protein, 16 aa, synthetic construct
  REGION 1..16: note = Synthetic Peptide
IIIIIHIHIHIHIHIH

SEQ ID NO: 167 - protein, 16 aa, synthetic construct
  REGION 1..16: note = Synthetic Peptide
MMMMMDMDMDMDMDMD

SEQ ID NO: 168 - protein, 16 aa, synthetic construct
  REGION 1..16: note = Synthetic Peptide
MMMMMEMEMEMEMEME

SEQ ID NO: 169 - protein, 16 aa, synthetic construct
  REGION 1..16: note = Synthetic Peptide
MMMMMKMKMKMKMKMK

SEQ ID NO: 170 - protein, 16 aa, synthetic construct
  REGION 1..16: note = Synthetic Peptide
MMMMMRMRMRMRMRMR

SEQ ID NO: 171 - protein, 16 aa, synthetic construct
  REGION 1..16: note = Synthetic Peptide
MMMMMHMHMHMHMHMH

SEQ ID NO: 172 - protein, 16 aa, synthetic construct
  REGION 1..16: note = Synthetic Peptide
FFFFFDFDFDFDFDFD

SEQ ID NO: 173 - protein, 16 aa, synthetic construct
  REGION 1..16: note = Synthetic Peptide
FFFFFEFEFEFEFEFE

SEQ ID NO: 174 - protein, 16 aa, synthetic construct
  REGION 1..16: note = Synthetic Peptide
FFFFFKFKFKFKFKFK

SEQ ID NO: 175 - protein, 16 aa, synthetic construct
  REGION 1..16: note = Synthetic Peptide
FFFFFRFRFRFRFRFR

SEQ ID NO: 176 - protein, 16 aa, synthetic construct
  REGION 1..16: note = Synthetic Peptide
FFFFFHFHFHFHFHFH

SEQ ID NO: 177 - protein, 16 aa, synthetic construct
  REGION 1..16: note = Synthetic Peptide
WWWWWDWDWDWDWDWD

SEQ ID NO: 178 - protein, 16 aa, synthetic construct
  REGION 1..16: note = Synthetic Peptide
WWWWWEWEWEWEWEWE

SEQ ID NO: 179 - protein, 16 aa, synthetic construct
  REGION 1..16: note = Synthetic Peptide
WWWWWKWKWKWKWKWK

SEQ ID NO: 180 - protein, 16 aa, synthetic construct
  REGION 1..16: note = Synthetic Peptide
WWWWWRWRWRWRWRWR

SEQ ID NO: 181 - protein, 16 aa, synthetic construct
  REGION 1..16: note = Synthetic Peptide
WWWWWHWHWHWHWHWH

SEQ ID NO: 182 - protein, 16 aa, synthetic construct
  REGION 1..16: note = Synthetic Peptide
PPPPPDPDPDPDPDPD

SEQ ID NO: 183 - protein, 16 aa, synthetic construct
  REGION 1..16: note = Synthetic Peptide
PPPPPEPEPEPEPEPE

SEQ ID NO: 184 - protein, 16 aa, synthetic construct
  REGION 1..16: note = Synthetic Peptide
PPPPPKPKPKPKPKPK

SEQ ID NO: 185 - protein, 16 aa, synthetic construct
  REGION 1..16: note = Synthetic Peptide
PPPPPRPRPRPRPRPR

SEQ ID NO: 186 - protein, 16 aa, synthetic construct
  REGION 1..16: note = Synthetic Peptide
PPPPPHPHPHPHPHPH

SEQ ID NO: 187 - protein, 16 aa, synthetic construct
  REGION 1..16: note = Synthetic Peptide
AAAAARADARADARAD

SEQ ID NO: 188 - protein, 16 aa, synthetic construct
  REGION 1..16: note = Synthetic Peptide
AAAAARARADADARAR

SEQ ID NO: 189 - protein, 16 aa, synthetic construct
  REGION 1..16: note = Synthetic Peptide
AAAAAEAKAEAKAEAK

SEQ ID NO: 190 - protein, 16 aa, synthetic construct
  REGION 1..16: note = Synthetic Peptide
AAAAAEAEAKAKAEAE

SEQ ID NO: 191 - protein, 16 aa, synthetic construct
  REGION 1..16: note = Synthetic Peptide
AAAAARAEARAEARAE

SEQ ID NO: 192 - protein, 16 aa, synthetic construct
  REGION 1..16: note = Synthetic Peptide
AAAAARARAEAEARAE

SEQ ID NO: 193 - protein, 16 aa, synthetic construct
  REGION 1..16: note = Synthetic Peptide
AAAAAKADAKADAKAD

SEQ ID NO: 194 - protein, 16 aa, synthetic construct
  REGION 1..16: note = Synthetic Peptide
AAAAAEAHAEAHAEAH

SEQ ID NO: 195 - protein, 16 aa, synthetic construct
  REGION 1..16: note = Synthetic Peptide
AAAAAEAEAHAHAEAE

SEQ ID NO: 196 - protein, 16 aa, synthetic construct
  REGION 1..16: note = Synthetic Peptide
AAAAARARARARADAD

SEQ ID NO: 197 - protein, 16 aa, synthetic construct
  REGION 1..16: note = Synthetic Peptide
AAAAARARARADADAD

SEQ ID NO: 198 - protein, 16 aa, synthetic construct
  REGION 1..16: note = Synthetic Peptide
AAAAAHADAHADAHAD

SEQ ID NO: 199 - protein, 16 aa, synthetic construct
  REGION 1..16: note = Synthetic Peptide
AAAAAHADADAHADAD

SEQ ID NO: 200 - protein, 16 aa, synthetic construct
  REGION 1..16: note = Synthetic Peptide
AAAAAHAEAEAHAEAE

SEQ ID NO: 201 - protein, 16 aa, synthetic construct
  REGION 1..16: note = Synthetic Peptide
GGGGGRGDGRGDGRGD

SEQ ID NO: 202 - protein, 16 aa, synthetic construct
  REGION 1..16: note = Synthetic Peptide
GGGGGRGRGDGDGRGR

SEQ ID NO: 203 - protein, 16 aa, synthetic construct
  REGION 1..16: note = Synthetic Peptide
GGGGGEGKGEGKGEGK

SEQ ID NO: 204 - protein, 16 aa, synthetic construct
  REGION 1..16: note = Synthetic Peptide
GGGGGEGEGKGKGEGE

SEQ ID NO: 205 - protein, 16 aa, synthetic construct
  REGION 1..16: note = Synthetic Peptide
GGGGGRGEGRGEGRGE

SEQ ID NO: 206 - protein, 16 aa, synthetic construct
  REGION 1..16: note = Synthetic Peptide
GGGGGRGRGEGEGRGE

SEQ ID NO: 207 - protein, 16 aa, synthetic construct
  REGION 1..16: note = Synthetic Peptide
GGGGGKGDGKGDGKGD

SEQ ID NO: 208 - protein, 16 aa, synthetic construct
  REGION 1..16: note = Synthetic Peptide
GGGGGEGHGEGHGEGH

SEQ ID NO: 209 - protein, 16 aa, synthetic construct
  REGION 1..16: note = Synthetic Peptide
GGGGGEGEGHGHGEGE

SEQ ID NO: 210 - protein, 16 aa, synthetic construct
  REGION 1..16: note = Synthetic Peptide
GGGGGRGRGRGRGDGD

SEQ ID NO: 211 - protein, 16 aa, synthetic construct
  REGION 1..16: note = Synthetic Peptide
GGGGGRGRGRGDGDGD

SEQ ID NO: 212 - protein, 16 aa, synthetic construct
  REGION 1..16: note = Synthetic Peptide
GGGGGHGDGHGDGHGD

SEQ ID NO: 213 - protein, 16 aa, synthetic construct
  REGION 1..16: note = Synthetic Peptide
GGGGGHGDGDGHGDGD

SEQ ID NO: 214 - protein, 16 aa, synthetic construct
  REGION 1..16: note = Synthetic Peptide
GGGGGHGEGEGHGEGE

SEQ ID NO: 215 - protein, 16 aa, synthetic construct
  REGION 1..16: note = Synthetic Peptide
VVVVVRVDVRVDVRVD

SEQ ID NO: 216 - protein, 16 aa, synthetic construct
  REGION 1..16: note = Synthetic Peptide
VVVVVRVRVDVDVRVR

SEQ ID NO: 217 - protein, 16 aa, synthetic construct
  REGION 1..16: note = Synthetic Peptide
VVVVVEVKVEVKVEVK

SEQ ID NO: 218 - protein, 16 aa, synthetic construct
  REGION 1..16: note = Synthetic Peptide
VVVVVEVEVKVKVEVE

SEQ ID NO: 219 - protein, 16 aa, synthetic construct
  REGION 1..16: note = Synthetic Peptide
VVVVVRVEVRVEVRVE

SEQ ID NO: 220 - protein, 16 aa, synthetic construct
  REGION 1..16: note = Synthetic Peptide
VVVVVRVRVEVEVRVE

SEQ ID NO: 221 - protein, 16 aa, synthetic construct
  REGION 1..16: note = Synthetic Peptide
VVVVVKVDVKVDVKVD

SEQ ID NO: 222 - protein, 16 aa, synthetic construct
  REGION 1..16: note = Synthetic Peptide
VVVVVEVHVEVHVEVH

SEQ ID NO: 223 - protein, 16 aa, synthetic construct
  REGION 1..16: note = Synthetic Peptide
VVVVVEVEVHVHVEVE

SEQ ID NO: 224 - protein, 16 aa, synthetic construct
  REGION 1..16: note = Synthetic Peptide
VVVVVRVRVRVRVDVD

SEQ ID NO: 225 - protein, 16 aa, synthetic construct
  REGION 1..16: note = Synthetic Peptide
VVVVVRVRVRVDVDVD

SEQ ID NO: 226 - protein, 16 aa, synthetic construct
  REGION 1..16: note = Synthetic Peptide
VVVVVHVDVHVDVHVD

SEQ ID NO: 227 - protein, 16 aa, synthetic construct
  REGION 1..16: note = Synthetic Peptide
VVVVVHVDVDVHVDVD

SEQ ID NO: 228 - protein, 16 aa, synthetic construct
  REGION 1..16: note = Synthetic Peptide
VVVVVHVEVEVHVEVE

SEQ ID NO: 229 - protein, 16 aa, synthetic construct
  REGION 1..16: note = Synthetic Peptide
LLLLLRLDLRLDLRLD

SEQ ID NO: 230 - protein, 16 aa, synthetic construct
  REGION 1..16: note = Synthetic Peptide
LLLLLRLRLDLDLRLR

SEQ ID NO: 231 - protein, 16 aa, synthetic construct
  REGION 1..16: note = Synthetic Peptide
LLLLLELKLELKLELK

SEQ ID NO: 232 - protein, 16 aa, synthetic construct
  REGION 1..16: note = Synthetic Peptide
LLLLLELELKLKLELE

SEQ ID NO: 233 - protein, 16 aa, synthetic construct
  REGION 1..16: note = Synthetic Peptide
LLLLLRLELRLELRLE

SEQ ID NO: 234 - protein, 16 aa, synthetic construct
  REGION 1..16: note = Synthetic Peptide
LLLLLRLRLELELRLE

SEQ ID NO: 235 - protein, 16 aa, synthetic construct
  REGION 1..16: note = Synthetic Peptide
LLLLLKLDLKLDLKLD

SEQ ID NO: 236 - protein, 16 aa, synthetic construct
  REGION 1..16: note = Synthetic Peptide
LLLLLELHLELHLELH

SEQ ID NO: 237 - protein, 16 aa, synthetic construct
  REGION 1..16: note = Synthetic Peptide
LLLLLELELHLHLELE

SEQ ID NO: 238 - protein, 16 aa, synthetic construct
  REGION 1..16: note = Synthetic Peptide
LLLLLRLRLRLRLDLD

SEQ ID NO: 239 - protein, 16 aa, synthetic construct
  REGION 1..16: note = Synthetic Peptide
LLLLLRLRLRLDLDLD

SEQ ID NO: 240 - protein, 16 aa, synthetic construct
  REGION 1..16: note = Synthetic Peptide
LLLLLHLDLHLDLHLD

SEQ ID NO: 241 - protein, 16 aa, synthetic construct
  REGION 1..16: note = Synthetic Peptide
LLLLLHLDLDLHLDLD

SEQ ID NO: 242 - protein, 16 aa, synthetic construct
  REGION 1..16: note = Synthetic Peptide
LLLLLHLELELHLELE

SEQ ID NO: 243 - protein, 16 aa, synthetic construct
  REGION 1..16: note = Synthetic Peptide
IIIIIRIDIRIDIRID

SEQ ID NO: 244 - protein, 16 aa, synthetic construct
  REGION 1..16: note = Synthetic Peptide
IIIIIRIRIDIDIRIR

SEQ ID NO: 245 - protein, 16 aa, synthetic construct
  REGION 1..16: note = Synthetic Peptide
IIIIIEIKIEIKIEIK

SEQ ID NO: 246 - protein, 16 aa, synthetic construct
  REGION 1..16: note = Synthetic Peptide
IIIIIEIEIKIKIEIE

SEQ ID NO: 247 - protein, 16 aa, synthetic construct
  REGION 1..16: note = Synthetic Peptide
IIIIIRIEIRIEIRIE

SEQ ID NO: 248 - protein, 16 aa, synthetic construct
  REGION 1..16: note = Synthetic Peptide
IIIIIRIRIEIEIRIE

SEQ ID NO: 249 - protein, 16 aa, synthetic construct
  REGION 1..16: note = Synthetic Peptide
IIIIIKIDIKIDIKID

SEQ ID NO: 250 - protein, 16 aa, synthetic construct
  REGION 1..16: note = Synthetic Peptide
IIIIIEIHIEIHIEIH

SEQ ID NO: 251 - protein, 16 aa, synthetic construct
  REGION 1..16: note = Synthetic Peptide
IIIIIEIEIHIHIEIE

SEQ ID NO: 252 - protein, 16 aa, synthetic construct
  REGION 1..16: note = Synthetic Peptide
IIIIIRIRIRIRIDID

SEQ ID NO: 253 - protein, 16 aa, synthetic construct
  REGION 1..16: note = Synthetic Peptide
IIIIIRIRIRIDIDID

SEQ ID NO: 254 - protein, 16 aa, synthetic construct
  REGION 1..16: note = Synthetic Peptide
IIIIIHIDIHIDIHID

SEQ ID NO: 255 - protein, 16 aa, synthetic construct
  REGION 1..16: note = Synthetic Peptide
IIIIIHIDIDIHIDID

SEQ ID NO: 256 - protein, 16 aa, synthetic construct
  REGION 1..16: note = Synthetic Peptide
IIIIIHIEIEIHIEIE

SEQ ID NO: 257 - protein, 16 aa, synthetic construct
  REGION 1..16: note = Synthetic Peptide
MMMMMRMDMRMDMRMD

SEQ ID NO: 258 - protein, 16 aa, synthetic construct
  REGION 1..16: note = Synthetic Peptide
MMMMMRMRMDMDMRMR

SEQ ID NO: 259 - protein, 16 aa, synthetic construct
  REGION 1..16: note = Synthetic Peptide
MMMMMEMKMEMKMEMK

SEQ ID NO: 260 - protein, 16 aa, synthetic construct
  REGION 1..16: note = Synthetic Peptide
MMMMMEMEMKMKMEME

SEQ ID NO: 261 - protein, 16 aa, synthetic construct
  REGION 1..16: note = Synthetic Peptide
MMMMMRMEMRMEMRME

SEQ ID NO: 262 - protein, 16 aa, synthetic construct
  REGION 1..16: note = Synthetic Peptide
MMMMMRMRMEMEMRME

SEQ ID NO: 263 - protein, 16 aa, synthetic construct
  REGION 1..16: note = Synthetic Peptide
MMMMMKMDMKMDMKMD

SEQ ID NO: 264 - protein, 16 aa, synthetic construct
  REGION 1..16: note = Synthetic Peptide
MMMMMEMHMEMHMEMH

SEQ ID NO: 265 - protein, 16 aa, synthetic construct
  REGION 1..16: note = Synthetic Peptide
MMMMMEMEMHMHMEME

SEQ ID NO: 266 - protein, 16 aa, synthetic construct
  REGION 1..16: note = Synthetic Peptide
MMMMMRMRMRMRMDMD

SEQ ID NO: 267 - protein, 16 aa, synthetic construct
  REGION 1..16: note = Synthetic Peptide
MMMMMRMRMRMDMDMD

SEQ ID NO: 268 - protein, 16 aa, synthetic construct
  REGION 1..16: note = Synthetic Peptide
MMMMMHMDMHMDMHMD

SEQ ID NO: 269 - protein, 16 aa, synthetic construct
  REGION 1..16: note = Synthetic Peptide
MMMMMHMDMDMHMDMD

SEQ ID NO: 270 - protein, 16 aa, synthetic construct
  REGION 1..16: note = Synthetic Peptide
MMMMMHMEMEMHMEME

SEQ ID NO: 271 - protein, 16 aa, synthetic construct
  REGION 1..16: note = Synthetic Peptide
FFFFFRFDFRFDFRFD

SEQ ID NO: 272 - protein, 16 aa, synthetic construct
  REGION 1..16: note = Synthetic Peptide
FFFFFRFRFDFDFRFR

SEQ ID NO: 273 - protein, 16 aa, synthetic construct
  REGION 1..16: note = Synthetic Peptide
FFFFFEFKFEFKFEFK

SEQ ID NO: 274 - protein, 16 aa, synthetic construct
  REGION 1..16: note = Synthetic Peptide
FFFFFEFEFKFKFEFE

SEQ ID NO: 275 - protein, 16 aa, synthetic construct
  REGION 1..16: note = Synthetic Peptide
FFFFFRFEFRFEFRFE

SEQ ID NO: 276 - protein, 16 aa, synthetic construct
  REGION 1..16: note = Synthetic Peptide
FFFFFRFRFEFEFRFE

SEQ ID NO: 277 - protein, 16 aa, synthetic construct
  REGION 1..16: note = Synthetic Peptide
FFFFFKFDFKFDFKFD

SEQ ID NO: 278 - protein, 16 aa, synthetic construct
  REGION 1..16: note = Synthetic Peptide
FFFFFEFHFEFHFEFH

SEQ ID NO: 279 - protein, 16 aa, synthetic construct
  REGION 1..16: note = Synthetic Peptide
FFFFFEFEFHFHFEFE

SEQ ID NO: 280 - protein, 16 aa, synthetic construct
  REGION 1..16: note = Synthetic Peptide
FFFFFRFRFRFRFDFD

SEQ ID NO: 281 - protein, 16 aa, synthetic construct
  REGION 1..16: note = Synthetic Peptide
FFFFFRFRFRFDFDFD

SEQ ID NO: 282 - protein, 16 aa, synthetic construct
  REGION 1..16: note = Synthetic Peptide
FFFFFHFDFHFDFHFD

SEQ ID NO: 283 - protein, 16 aa, synthetic construct
  REGION 1..16: note = Synthetic Peptide
FFFFFHFDFDFHFDFD

SEQ ID NO: 284 - protein, 16 aa, synthetic construct
  REGION 1..16: note = Synthetic Peptide
FFFFFHFEFEFHFEFE

SEQ ID NO: 285 - protein, 16 aa, synthetic construct
  REGION 1..16: note = Synthetic Peptide
WWWWWRWDWRWDWRWD

SEQ ID NO: 286 - protein, 16 aa, synthetic construct
  REGION 1..16: note = Synthetic Peptide
WWWWWRWRWDWDWRWR

SEQ ID NO: 287 - protein, 16 aa, synthetic construct
  REGION 1..16: note = Synthetic Peptide
WWWWWEWKWEWKWEWK

SEQ ID NO: 288 - protein, 16 aa, synthetic construct
  REGION 1..16: note = Synthetic Peptide
WWWWWEWEWKWKWEWE

SEQ ID NO: 289 - protein, 16 aa, synthetic construct
  REGION 1..16: note = Synthetic Peptide
WWWWWRWEWRWEWRWE

SEQ ID NO: 290 - protein, 16 aa, synthetic construct
  REGION 1..16: note = Synthetic Peptide
WWWWWRWRWEWEWRWE

SEQ ID NO: 291 - protein, 16 aa, synthetic construct
  REGION 1..16: note = Synthetic Peptide
WWWWWKWDWKWDWKWD

SEQ ID NO: 292 - protein, 16 aa, synthetic construct
  REGION 1..16: note = Synthetic Peptide
WWWWWEWHWEWHWEWH

SEQ ID NO: 293 - protein, 16 aa, synthetic construct
  REGION 1..16: note = Synthetic Peptide
WWWWWEWEWHWHWEWE

SEQ ID NO: 294 - protein, 16 aa, synthetic construct
  REGION 1..16: note = Synthetic Peptide
WWWWWRWRWRWRWDWD

SEQ ID NO: 295 - protein, 16 aa, synthetic construct
  REGION 1..16: note = Synthetic Peptide
WWWWWRWRWRWDWDWD

SEQ ID NO: 296 - protein, 16 aa, synthetic construct
  REGION 1..16: note = Synthetic Peptide
WWWWWHWDWHWDWHWD

SEQ ID NO: 297 - protein, 16 aa, synthetic construct
  REGION 1..16: note = Synthetic Peptide
WWWWWHWDWDWHWDWD

SEQ ID NO: 298 - protein, 16 aa, synthetic construct
  REGION 1..16: note = Synthetic Peptide
WWWWWHWEWEWHWEWE

SEQ ID NO: 299 - protein, 16 aa, synthetic construct
  REGION 1..16: note = Synthetic Peptide
PPPPPRPDPRPDPRPD

SEQ ID NO: 300 - protein, 16 aa, synthetic construct
  REGION 1..16: note = Synthetic Peptide
PPPPPRPRPDPDPRPR

SEQ ID NO: 301 - protein, 16 aa, synthetic construct
  REGION 1..16: note = Synthetic Peptide
PPPPPEPKPEPKPEPK

SEQ ID NO: 302 - protein, 16 aa, synthetic construct
  REGION 1..16: note = Synthetic Peptide
PPPPPEPEPKPKPEPE

SEQ ID NO: 303 - protein, 16 aa, synthetic construct
  REGION 1..16: note = Synthetic Peptide
PPPPPRPEPRPEPRPE

SEQ ID NO: 304 - protein, 16 aa, synthetic construct
  REGION 1..16: note = Synthetic Peptide
PPPPPRPRPEPEPRPE

SEQ ID NO: 305 - protein, 16 aa, synthetic construct
  REGION 1..16: note = Synthetic Peptide
PPPPPKPDPKPDPKPD

SEQ ID NO: 306 - protein, 16 aa, synthetic construct
  REGION 1..16: note = Synthetic Peptide
PPPPPEPHPEPHPEPH

SEQ ID NO: 307 - protein, 16 aa, synthetic construct
  REGION 1..16: note = Synthetic Peptide
PPPPPEPEPHPHPEPE

SEQ ID NO: 308 - protein, 16 aa, synthetic construct
  REGION 1..16: note = Synthetic Peptide
PPPPPRPRPRPRPDPD

SEQ ID NO: 309 - protein, 16 aa, synthetic construct
  REGION 1..16: note = Synthetic Peptide
PPPPPRPRPRPDPDPD

SEQ ID NO: 310 - protein, 16 aa, synthetic construct
  REGION 1..16: note = Synthetic Peptide
PPPPPHPDPHPDPHPD

SEQ ID NO: 311 - protein, 16 aa, synthetic construct
  REGION 1..16: note = Synthetic Peptide
PPPPPHPDPDPHPDPD

SEQ ID NO: 312 - protein, 16 aa, synthetic construct
  REGION 1..16: note = Synthetic Peptide
PPPPPHPEPEPHPEPE

SEQ ID NO: 313 - protein, 16 aa, synthetic construct
  REGION 1..16: note = Synthetic Peptide
SSSSSRSDSRSDSRSD

SEQ ID NO: 314 - protein, 16 aa, synthetic construct
  REGION 1..16: note = Synthetic Peptide
SSSSSRSRSDSDSRSR

SEQ ID NO: 315 - protein, 16 aa, synthetic construct
  REGION 1..16: note = Synthetic Peptide
SSSSSESKSESKSESK

SEQ ID NO: 316 - protein, 16 aa, synthetic construct
  REGION 1..16: note = Synthetic Peptide
SSSSSESESKSKSESE

SEQ ID NO: 317 - protein, 16 aa, synthetic construct
  REGION 1..16: note = Synthetic Peptide
SSSSSRSESRSESRSE

SEQ ID NO: 318 - protein, 16 aa, synthetic construct
  REGION 1..16: note = Synthetic Peptide
SSSSSRSRSESESRSE

SEQ ID NO: 319 - protein, 16 aa, synthetic construct
  REGION 1..16: note = Synthetic Peptide
SSSSSKSDSKSDSKSD

SEQ ID NO: 320 - protein, 16 aa, synthetic construct
  REGION 1..16: note = Synthetic Peptide
SSSSSESHSESHSESH

SEQ ID NO: 321 - protein, 16 aa, synthetic construct
  REGION 1..16: note = Synthetic Peptide
SSSSSESESHSHSESE

SEQ ID NO: 322 - protein, 16 aa, synthetic construct
  REGION 1..16: note = Synthetic Peptide
SSSSSRSRSRSRSRSR

SEQ ID NO: 323 - protein, 16 aa, synthetic construct
  REGION 1..16: note = Synthetic Peptide
SSSSSRSRSRSRSDSD

SEQ ID NO: 324 - protein, 16 aa, synthetic construct
  REGION 1..16: note = Synthetic Peptide
SSSSSRSRSRSDSDSD

SEQ ID NO: 325 - protein, 16 aa, synthetic construct
  REGION 1..16: note = Synthetic Peptide
SSSSSHSDSHSDSHSD

SEQ ID NO: 326 - protein, 16 aa, synthetic construct
  REGION 1..16: note = Synthetic Peptide
SSSSSHSHSHSHSHSH

SEQ ID NO: 327 - protein, 16 aa, synthetic construct
  REGION 1..16: note = Synthetic Peptide
SSSSSHSDSDSHSDSD

SEQ ID NO: 328 - protein, 16 aa, synthetic construct
  REGION 1..16: note = Synthetic Peptide
SSSSSHSESESHSESE

SEQ ID NO: 329 - protein, 16 aa, synthetic construct
  REGION 1..16: note = Synthetic Peptide
TTTTTRTDTRTDTRTD

SEQ ID NO: 330 - protein, 16 aa, synthetic construct
  REGION 1..16: note = Synthetic Peptide
TTTTTRTRTDTDTRTR

SEQ ID NO: 331 - protein, 16 aa, synthetic construct
  REGION 1..16: note = Synthetic Peptide
TTTTTETKTETKTETK

SEQ ID NO: 332 - protein, 16 aa, synthetic construct
  REGION 1..16: note = Synthetic Peptide
TTTTTETETKTKTETE

SEQ ID NO: 333 - protein, 16 aa, synthetic construct
  REGION 1..16: note = Synthetic Peptide
TTTTTRTETRTETRTE

SEQ ID NO: 334 - protein, 16 aa, synthetic construct
  REGION 1..16: note = Synthetic Peptide
TTTTTRTRTETETRTE

SEQ ID NO: 335 - protein, 16 aa, synthetic construct
  REGION 1..16: note = Synthetic Peptide
TTTTTKTDTKTDTKTD

SEQ ID NO: 336 - protein, 16 aa, synthetic construct
  REGION 1..16: note = Synthetic Peptide
TTTTTETHTETHTETH

SEQ ID NO: 337 - protein, 16 aa, synthetic construct
  REGION 1..16: note = Synthetic Peptide
TTTTTETETHTHTETE

SEQ ID NO: 338 - protein, 16 aa, synthetic construct
  REGION 1..16: note = Synthetic Peptide
TTTTTRTRTRTRTRTR

SEQ ID NO: 339 - protein, 16 aa, synthetic construct
  REGION 1..16: note = Synthetic Peptide
TTTTTRTRTRTRTDTD

SEQ ID NO: 340 - protein, 16 aa, synthetic construct
  REGION 1..16: note = Synthetic Peptide
TTTTTRTRTRTDTDTD

SEQ ID NO: 341 - protein, 16 aa, synthetic construct
  REGION 1..16: note = Synthetic Peptide
TTTTTHTDTHTDTHTD

SEQ ID NO: 342 - protein, 16 aa, synthetic construct
  REGION 1..16: note = Synthetic Peptide
TTTTTHTHTHTHTHTH

SEQ ID NO: 343 - protein, 16 aa, synthetic construct
  REGION 1..16: note = Synthetic Peptide
TTTTTHTDTDTHTDTD

SEQ ID NO: 344 - protein, 16 aa, synthetic construct
  REGION 1..16: note = Synthetic Peptide
TTTTTHTETETHTETE

SEQ ID NO: 345 - protein, 16 aa, synthetic construct
  REGION 1..16: note = Synthetic Peptide
CCCCCRCDCRCDCRCD

SEQ ID NO: 346 - protein, 16 aa, synthetic construct
  REGION 1..16: note = Synthetic Peptide
CCCCCRCRCDCDCRCR

SEQ ID NO: 347 - protein, 16 aa, synthetic construct
  REGION 1..16: note = Synthetic Peptide
CCCCCECKCECKCECK

SEQ ID NO: 348 - protein, 16 aa, synthetic construct
  REGION 1..16: note = Synthetic Peptide
CCCCCECECKCKCECE

SEQ ID NO: 349 - protein, 16 aa, synthetic construct
  REGION 1..16: note = Synthetic Peptide
CCCCCRCECRCECRCE

SEQ ID NO: 350 - protein, 16 aa, synthetic construct
  REGION 1..16: note = Synthetic Peptide
CCCCCRCRCECECRCE

SEQ ID NO: 351 - protein, 16 aa, synthetic construct
  REGION 1..16: note = Synthetic Peptide
CCCCCKCDCKCDCKCD

SEQ ID NO: 352 - protein, 16 aa, synthetic construct
  REGION 1..16: note = Synthetic Peptide
CCCCCECHCECHCECH

SEQ ID NO: 353 - protein, 16 aa, synthetic construct
  REGION 1..16: note = Synthetic Peptide
CCCCCECECHCHCECE

SEQ ID NO: 354 - protein, 16 aa, synthetic construct
  REGION 1..16: note = Synthetic Peptide
CCCCCRCRCRCRCRCR

SEQ ID NO: 355 - protein, 16 aa, synthetic construct
  REGION 1..16: note = Synthetic Peptide
CCCCCRCRCRCRCDCD

SEQ ID NO: 356 - protein, 16 aa, synthetic construct
  REGION 1..16: note = Synthetic Peptide
CCCCCRCRCRCDCDCD

SEQ ID NO: 357 - protein, 16 aa, synthetic construct
  REGION 1..16: note = Synthetic Peptide
CCCCCHCDCHCDCHCD

SEQ ID NO: 358 - protein, 16 aa, synthetic construct
  REGION 1..16: note = Synthetic Peptide
CCCCCHCHCHCHCHCH

SEQ ID NO: 359 - protein, 16 aa, synthetic construct
  REGION 1..16: note = Synthetic Peptide
CCCCCHCDCDCHCDCD

SEQ ID NO: 360 - protein, 16 aa, synthetic construct
  REGION 1..16: note = Synthetic Peptide
CCCCCHCECECHCECE

SEQ ID NO: 361 - protein, 16 aa, synthetic construct
  REGION 1..16: note = Synthetic Peptide
YYYYYRYDYRYDYRYD

SEQ ID NO: 362 - protein, 16 aa, synthetic construct
  REGION 1..16: note = Synthetic Peptide
YYYYYRYRYDYDYRYR

SEQ ID NO: 363 - protein, 16 aa, synthetic construct
  REGION 1..16: note = Synthetic Peptide
YYYYYEYKYEYKYEYK

SEQ ID NO: 364 - protein, 16 aa, synthetic construct
  REGION 1..16: note = Synthetic Peptide
YYYYYEYEYKYKYEYE

SEQ ID NO: 365 - protein, 16 aa, synthetic construct
  REGION 1..16: note = Synthetic Peptide
YYYYYRYEYRYEYRYE

SEQ ID NO: 366 - protein, 16 aa, synthetic construct
  REGION 1..16: note = Synthetic Peptide
YYYYYRYRYEYEYRYE

SEQ ID NO: 367 - protein, 16 aa, synthetic construct
  REGION 1..16: note = Synthetic Peptide
YYYYYKYDYKYDYKYD

SEQ ID NO: 368 - protein, 16 aa, synthetic construct
  REGION 1..16: note = Synthetic Peptide
YYYYYEYHYEYHYEYH

SEQ ID NO: 369 - protein, 16 aa, synthetic construct
  REGION 1..16: note = Synthetic Peptide
YYYYYEYEYHYHYEYE

SEQ ID NO: 370 - protein, 16 aa, synthetic construct
  REGION 1..16: note = Synthetic Peptide
YYYYYRYRYRYRYRYR

SEQ ID NO: 371 - protein, 16 aa, synthetic construct
  REGION 1..16: note = Synthetic Peptide
YYYYYRYRYRYRYDYD

SEQ ID NO: 372 - protein, 16 aa, synthetic construct
  REGION 1..16: note = Synthetic Peptide
YYYYYRYRYRYDYDYD

SEQ ID NO: 373 - protein, 16 aa, synthetic construct
  REGION 1..16: note = Synthetic Peptide
YYYYYHYDYHYDYHYD

SEQ ID NO: 374 - protein, 16 aa, synthetic construct
  REGION 1..16: note = Synthetic Peptide
YYYYYHYHYHYHYHYH

SEQ ID NO: 375 - protein, 16 aa, synthetic construct
  REGION 1..16: note = Synthetic Peptide
YYYYYHYDYDYHYDYD

SEQ ID NO: 376 - protein, 16 aa, synthetic construct
  REGION 1..16: note = Synthetic Peptide
YYYYYHYEYEYHYEYE

SEQ ID NO: 377 - protein, 16 aa, synthetic construct
  REGION 1..16: note = Synthetic Peptide
NNNNNRNDNRNDNRND

SEQ ID NO: 378 - protein, 16 aa, synthetic construct
  REGION 1..16: note = Synthetic Peptide
NNNNNRNRNDNDNRNR

SEQ ID NO: 379 - protein, 16 aa, synthetic construct
  REGION 1..16: note = Synthetic Peptide
NNNNNENKNENKNENK

SEQ ID NO: 380 - protein, 16 aa, synthetic construct
  REGION 1..16: note = Synthetic Peptide
NNNNNENENKNKNENE

SEQ ID NO: 381 - protein, 16 aa, synthetic construct
  REGION 1..16: note = Synthetic Peptide
NNNNNRNENRNENRNE

SEQ ID NO: 382 - protein, 16 aa, synthetic construct
  REGION 1..16: note = Synthetic Peptide
NNNNNRNRNENENRNE

SEQ ID NO: 383 - protein, 16 aa, synthetic construct
  REGION 1..16: note = Synthetic Peptide
NNNNNKNDNKNDNKND

SEQ ID NO: 384 - protein, 16 aa, synthetic construct
  REGION 1..16: note = Synthetic Peptide
NNNNNENHNENHNENH

SEQ ID NO: 385 - protein, 16 aa, synthetic construct
  REGION 1..16: note = Synthetic Peptide
NNNNNENENHNHNENE

SEQ ID NO: 386 - protein, 16 aa, synthetic construct
  REGION 1..16: note = Synthetic Peptide
NNNNNRNRNRNRNRNR

SEQ ID NO: 387 - protein, 16 aa, synthetic construct
  REGION 1..16: note = Synthetic Peptide
NNNNNRNRNRNRNDND

SEQ ID NO: 388 - protein, 16 aa, synthetic construct
  REGION 1..16: note = Synthetic Peptide
NNNNNRNRNRNDNDND

SEQ ID NO: 389 - protein, 16 aa, synthetic construct
  REGION 1..16: note = Synthetic Peptide
NNNNNHNDNHNDNHND

SEQ ID NO: 390 - protein, 16 aa, synthetic construct
  REGION 1..16: note = Synthetic Peptide
NNNNNHNHNHNHNHNH

SEQ ID NO: 391 - protein, 16 aa, synthetic construct
  REGION 1..16: note = Synthetic Peptide
NNNNNHNDNDNHNDND

SEQ ID NO: 392 - protein, 16 aa, synthetic construct
  REGION 1..16: note = Synthetic Peptide
NNNNNHNENENHNENE

SEQ ID NO: 393 - protein, 16 aa, synthetic construct
  REGION 1..16: note = Synthetic Peptide
QQQQQRQDQRQDQRQD

SEQ ID NO: 394 - protein, 16 aa, synthetic construct
  REGION 1..16: note = Synthetic Peptide
QQQQQRQRQDQDQRQR

SEQ ID NO: 395 - protein, 16 aa, synthetic construct
  REGION 1..16: note = Synthetic Peptide
QQQQQEQKQEQKQEQK

SEQ ID NO: 396 - protein, 16 aa, synthetic construct
  REGION 1..16: note = Synthetic Peptide
QQQQQEQEQKQKQEQE

SEQ ID NO: 397 - protein, 16 aa, synthetic construct
  REGION 1..16: note = Synthetic Peptide
QQQQQRQEQRQEQRQE

SEQ ID NO: 398 - protein, 16 aa, synthetic construct
  REGION 1..16: note = Synthetic Peptide
QQQQQRQRQEQEQRQE

SEQ ID NO: 399 - protein, 16 aa, synthetic construct
  REGION 1..16: note = Synthetic Peptide
QQQQQKQDQKQDQKQD

SEQ ID NO: 400 - protein, 16 aa, synthetic construct
  REGION 1..16: note = Synthetic Peptide
QQQQQEQHQEQHQEQH

SEQ ID NO: 401 - protein, 16 aa, synthetic construct
  REGION 1..16: note = Synthetic Peptide
QQQQQEQEQHQHQEQE

SEQ ID NO: 402 - protein, 16 aa, synthetic construct
  REGION 1..16: note = Synthetic Peptide
QQQQQRQRQRQRQRQR

SEQ ID NO: 403 - protein, 16 aa, synthetic construct
  REGION 1..16: note = Synthetic Peptide
QQQQQRQRQRQRQDQD

SEQ ID NO: 404 - protein, 16 aa, synthetic construct
  REGION 1..16: note = Synthetic Peptide
QQQQQRQRQRQDQDQD

SEQ ID NO: 405 - protein, 16 aa, synthetic construct
  REGION 1..16: note = Synthetic Peptide
QQQQQHQDQHQDQHQD

SEQ ID NO: 406 - protein, 16 aa, synthetic construct
  REGION 1..16: note = Synthetic Peptide
QQQQQHQHQHQHQHQH

SEQ ID NO: 407 - protein, 16 aa, synthetic construct
  REGION 1..16: note = Synthetic Peptide
QQQQQHQDQDQHQDQD

SEQ ID NO: 408 - protein, 16 aa, synthetic construct
  REGION 1..16: note = Synthetic Peptide
QQQQQHQEQEQHQEQE

SEQ ID NO: 409 - protein, 8 aa, synthetic construct
  REGION 1..8: note = Synthetic Peptide
  SITE 1: note = MISC_FEATURE - Pyridoxamine phosphate group
  MOD_RES 1: note = Methylated tyrosine
  MOD_RES 7: note = X = Ornithine
  SITE 8: note = C-terminal amine (NH2)
YITNCPXY

SEQ ID NO: 410 - protein, 7 aa, synthetic construct
  REGION 1..7: note = Synthetic Peptide
  SITE 1: note = MISC_FEATURE - 3-mercaptopropionyl group
YFQNCPR

SEQ ID NO: 411 - protein, 9 aa, synthetic construct
  REGION 1..9: note = Synthetic Peptide
  SITE 9: note = C-terminal amine (NH2)
CYFQNCPRG

SEQ ID NO: 412 - protein, 8 aa, synthetic construct
  REGION 1..8: note = Synthetic Peptide
CYFQNCPR

SEQ ID NO: 413 - protein, 20 aa, synthetic construct
  REGION 1..20: note = Synthetic Peptide
  MOD_RES 1: note = Acetylated Arginine
  SITE 20: note = MISC_FEATURE - C-terminal amide
RADARADARADACVSVPQAL

SEQ ID NO: 414 - protein, 12 aa, synthetic construct
  REGION 1..12: note = Synthetic Peptide
RARARADADADA

SEQ ID NO: 415 - protein, 16 aa, synthetic construct
  REGION 1..16: note = Synthetic Peptide
RARARARADADADADA

SEQ ID NO: 416 - protein, 8 aa, synthetic construct
  REGION 1..8: note = Synthetic Peptide
RRRRDDDD

SEQ ID NO: 417 - protein, 8 aa, synthetic construct
  REGION 1..8: note = Synthetic Peptide
GGGGSSSS

SEQ ID NO: 418 - protein, 4 aa, synthetic construct
  REGION 1..4: note = Synthetic Peptide
KKKK

SEQ ID NO: 419 - protein, 4 aa, synthetic construct
  REGION 1..4: note = Synthetic Peptide
RRRR

SEQ ID NO: 420 - protein, 4 aa, synthetic construct
  REGION 1..4: note = Synthetic Peptide
HHHH

SEQ ID NO: 421 - protein, 4 aa, synthetic construct
  REGION 1..4: note = Synthetic Peptide
DDDD

SEQ ID NO: 422 - protein, 4 aa, synthetic construct
  REGION 1..4: note = Synthetic Peptide
EEEE

SEQ ID NO: 423 - protein, 4 aa, synthetic construct
  REGION 1..4: note = Synthetic Peptide
GQGQ

SEQ ID NO: 424 - protein, 6 aa, synthetic construct
  REGION 1..6: note = Synthetic Peptide
GGQQGG

SEQ ID NO: 425 - protein, 7 aa, synthetic construct
  REGION 1..7: note = Synthetic Peptide
GQQGQQG

SEQ ID NO: 426 - protein, 8 aa, synthetic construct
  REGION 1..8: note = Synthetic Peptide
GGQGGQGG

SEQ ID NO: 427 - protein, 16 aa, synthetic construct
  REGION 1..16: note = Synthetic Peptide
  SITE 2: note = beta alanine
  SITE 4: note = beta alanine
  SITE 6: note = beta alanine
  SITE 8: note = beta alanine
  SITE 10: note = beta alanine
  SITE 12: note = beta alanine
  SITE 14: note = beta alanine
  SITE 16: note = beta alanine
EAKAEAKAEAKAEAKA

SEQ ID NO: 428 - protein, 8 aa, synthetic construct
  REGION 1..8: note = Synthetic Peptide
  SITE 2: note = beta alanine
  SITE 4: note = beta alanine
  SITE 6: note = beta alanine
  SITE 8: note = beta alanine
EAKAEAKA

SEQ ID NO: 429 - protein, 16 aa, synthetic construct
  REGION 1..16: note = Synthetic Peptide
  SITE 2: note = beta alanine
  SITE 4: note = beta alanine
  SITE 6: note = beta alanine
  SITE 8: note = beta alanine
  SITE 10: note = beta alanine
  SITE 12: note = beta alanine
  SITE 14: note = beta alanine
  SITE 16: note = beta alanine
RADARADARADARADA

SEQ ID NO: 430 - protein, 8 aa, synthetic construct
  REGION 1..8: note = Synthetic Peptide
  SITE 2: note = beta alanine
  SITE 4: note = beta alanine
  SITE 6: note = beta alanine
  SITE 8: note = beta alanine
RADARADA

(19) United States
(12) Patent Application Publication
(10) Pub. No.: US 2024/0051997 A1
(43) Pub. Date: Feb. 15, 2024

(54) SAP AND PEPTIDOMIMETIC COMPOSITIONS FOR REDUCING SYMPTOMS OF INFLAMMATION

(71) Applicant: Arch Biosurgery, Inc., Framington, MA (US)

(72) Inventors: Terrence Norchi, Natick, MA (US); Rutledge Ellis-Behnke, Myrtle Beach, SC (US)

(21) Appl. No.: 18/074,227

(22) Filed: Dec. 2, 2022

Related U.S. Application Data
(63) Continuation of application No. 16/363,890, filed on Mar. 25, 2019, now abandoned.
(60) Provisional application No. 62/647,082, filed on Mar. 23, 2018.

(51) Int. Cl. C07K 14/00; A61P 29/00; A61P 19/02; A61K 9/00; C07K 2/00; C07K 7/06; C07K 7/08; A61K 9/16; A61P 11/06; A61K 38/03; A61K 38/10; A61K 38/08; A61K 38/16; A61K 38/07

(52) U.S. Cl. CPC C07K 14/001; A61P 29/00; A61P 19/02; A61K 9/0019; A61K 9/0014; C07K 2/00; C07K 7/06; C07K 7/08; A61K 9/1658; A61P 11/06; A61K 38/03; A61K 38/10; A61K 38/08; A61K 38/16; A61K 38/07; A61K 38/00

(57) ABSTRACT

Self-assembling peptides or self-assembling peptidomimetics (“SAP”) can treat inflammation or inflammatory diseases, or reduce one or more symptoms of diseases and disorders associated with undesirable inflammation. Topical and injectable compositions of SAP for local administration to a site of inflammation for reduction or prevention of symptoms of inflammatory diseases and disorders are described. The compositions include one or more SAP in an amount and concentration effective to reduce or prevent one or more symptoms of undesirable inflammation. The SAP can assemble prior to or after the composition is administered. The SAP form a structure within or at the surface of the body that prevents and/or reduces symptoms associated with inflammation and other dysregulated immune processes. The peptides can assemble upon contact with bodily fluids (e.g., synovial fluid), or can be contacted with ionic solutions to initiate assembly.

### CROSS-REFERENCE TO RELATED APPLICATIONS

This application is a continuation of U.S. Ser. No. 16/363,890, filed Mar. 25, 2019, which claims the benefit of and priority to U.S. Ser. No. 62/647,082 filed Mar. 23, 2018, and which is incorporated by reference in its entirety.

### REFERENCE TO SEQUENCE LISTING

The Sequence Listing submitted as a text file named “CNS_110_CON_ST26.xml” created on Oct. 24, 2023, and having a size of 579,898 bytes is hereby incorporated by reference pursuant to 37 C.F.R. § 1.834(c)(1).

### FIELD OF THE INVENTION

This invention is in the field of therapeutic reagents, particularly compositions of SAPs which control (e.g., reduce or prevent) undesired signs or symptoms of inflammation and inflammatory diseases and disorders.

### BACKGROUND OF THE INVENTION

Suppression of unwanted symptoms of inflammation resulting from injury, diseases and surgery can be of high clinical importance in many diseases. Inflammation is the physiologic response of the body to harmful stimuli, such as pathogens, damaged cells, or irritants. As part of a complex biological reaction, inflammation is a protective attempt by the organism to remove the injurious stimuli and to initiate the healing process. Inflammation is thus an essential component of the healing process of wounds and infections.

Cytokines mediate and control immune and inflammatory responses. Complex interactions between cytokines, inflammation and the adaptive responses maintain homeostasis. The inflammatory reaction is crucial for survival and is meant to be tailored to the stimulus and time. Systemic inflammatory reactions can be categorized in four major reactions, including the acute-phase reaction, the sickness syndrome, the pain program, and the stress response, mediated by the hypothalamic-pituitary-adrenal axis and the sympathetic nervous system. Common human diseases such as atopy/allergy, autoimmunity, chronic infections and sepsis are characterized by a dysregulation of the pro-versus anti-inflammatory and T helper 1 (Th1) versus Th2 cytokine balance. Recent evidence also indicates the involvement of pro-inflammatory cytokines in the pathogenesis of atherosclerosis and major depression, and conditions such as visceral-type obesity, metabolic syndrome and sleep disturbances. During inflammation, the activation of the stress system, through induction of a Th2 shift, protects the organism from systemic ‘overshooting’ with Th1/pro-inflammatory cytokines. Under certain conditions, however, stress hormones may actually facilitate inflammation through induction of interleukin (IL)-1, IL-6, IL-8, IL-18, tumor necrosis factor-alpha and C-reactive protein production and through activation of the corticotropin-releasing hormone/substance P-histamine axis. Thus, a dysfunctional neuroendocrine-immune interface associated with abnormalities of the ‘systemic anti-inflammatory feedback’ and/or ‘hyperactivity’ of the local pro-inflammatory factors may play a role in the pathogenesis of atopic/allergic and autoimmune diseases, obesity, depression, and atherosclerosis. These abnormalities and the failure of the adaptive systems to resolve inflammation affect the well-being of the individual, including behavioral parameters, quality of life and sleep, as well as indices of metabolic and cardiovascular health.

Overshooting inflammation, particularly if it becomes chronic, can also lead to, or can be associated with, a full panoply of symptoms associated with diseases such as rheumatoid arthritis and atherosclerosis (Choy and Panayi, N Engl J Med. March 22; 344(12):907-16 (2001); Hansson, N Engl J Med. April 21; 352(16):1685-95 (2005); and Ross, N Engl J Med. January 14; 340(2):115-26 (1999)). Medical regimens which so far have widely been applied in clinical medicine have usually been based on steroids and non-steroidal anti-inflammatory drugs (Rhen, N Engl J Med., 353(16):1711-23 (2005); and Simon and Mills, N Engl J Med., 302(22):1237-43 (1980)). Both groups of drugs, however, can show serious side-effects, such as the full spectrum of Cushing's syndrome if steroids are given, and gastric ulcerations if non-steroidal inflammatory drugs are applied.

There remains a need for therapies for mitigating the symptoms of unwanted inflammation and inflammatory diseases and disorders, including autoimmune diseases and disorders.

It is therefore an object of the present invention to provide compositions for reducing or preventing one or more of the signs or symptoms of inflammation in the absence of undesirable side effects.

It is also an object of the present invention to provide methods and compositions for reducing or preventing the symptoms of chronic inflammatory disorders and diseases.

It is also an object of the present invention to provide methods and compositions for reducing or preventing symptoms of host-versus-graft disease, graft-versus-host disease and transplant rejection.

It is still a further object of the present invention to provide methods and compositions for reducing and preventing symptoms of auto-immune diseases, and symptoms of disorders associated with dis-regulated immune cell functions.

### SUMMARY OF THE INVENTION

Compositions of self-assembling peptides and/or self-assembling peptidomimetics (referred to herein as “SAP”, unless designated otherwise), and methods of use thereof for reducing or preventing one or more signs or symptoms of inflammation and/or inflammatory diseases by application directly onto or into the site of inflammation, are described. Symptoms that may be reduced or prevented include pain, irritation, swelling, redness or other discoloration, loss of sensation, reduced mobility, fever, headache, itching, discharge of pus, headache, chills, muscle stiffness, immobility of a joint, loss of function of an organ, stimulation of nerve endings by bradykinin, increased blood flow, malaise, and physiological responses associated with production of histamine and/or heparin.

The SAP may be assembled prior to application or may be applied as non-assembled precursor peptides and/or peptidomimetics, which assemble during or following application. Assembly can be initiated upon contact with bodily fluids (e.g., blood or serum), or upon contact with an ionic solution.

In some embodiments the SAP have a sequence of amino acid residues conforming to one or more of the following formulas:

((Xaa^{neu}−Xaa⁺)ₓ(Xaa^{neu}−Xaa⁻)_{y})ₙ; (I)

((Xaa^{neu}−Xaa⁻)ₓ(Xaa^{neu}−Xaa⁺)_{y})ₙ; (II)

((Xaa⁺−Xaa^{neu})ₓ(Xaa⁻−Xaa^{neu})_{y})ₙ; (III) and

((Xaa⁻−Xaa^{neu})ₓ(Xaa⁺−Xaa^{neu})_{y})ₙ, (IV)

where each Xaa^{neu} represents an amino acid residue having a neutral charge, Xaa⁺ represents an amino acid residue having a positive charge, Xaa⁻ represents an amino acid residue having a negative charge, x and y are integers having a value of 1, 2, 3, or 4, independently, and n is an integer having a value of 1-5.

In certain embodiments, up to 100% of the SAP in the composition are of the same size and have the same amino acid sequence, for example, 75% or more, such as 80%, 85%, 90% or 95%, or 99% of the SAP are of the same size and have the same amino acid sequence. In other embodiments, compositions include two or more different SAP, having different sizes and sequences. The compositions can also include polymers and can be partly biodegradable, fully biodegradable, or non-biodegradable. Compositions can optionally include a scaffold or support material.

Compositions including one or more SAP may contain one or more agents, such as live cells, therapeutic agents, prophylactic agents, and/or diagnostic agents. Examples include non-self-assembling anti-inflammatory drugs, local anesthetics, antimicrobial agents, anti-angiogenesis agents, immunosuppressants, chemotherapeutic agents, anti-hypertensive drugs, and combinations thereof. In other embodiments, the agent is a pH-adjusting agent, a dye, a filler, or combinations thereof. The agent can be covalently or non-covalently bound to the SAP. The compositions are suitable for administration into the bloodstream or onto one or more surfaces of the body, for example, by instillation or injection. In preferred embodiments, compositions of SAP are formulated for administration directly onto tissue by topical administration, or into the interior structure(s) of an organ by injection.

Bandages and other support structures incorporating or otherwise associated with SAP are also described. The bandages or support structures can include one or more therapeutic agents, prophylactic agents, or diagnostic agents. The backing material or support scaffold can be a non-peptide material.

Methods of making compositions that contain SAP for administration to the body for reduction or prevention of one or more symptoms of inflammation are also provided. The methods can include injection molding, stamping, templating onto a surface having a desired shape, coating of a solid substrate, electro-spinning, or combinations of these. The SAP can be assembled by contacting the composition with a solution of cations. Self-assembly of the peptides can occur at the time of manufacture of the composition, or immediately prior to, during or after application of the composition to the body.

Methods for reducing or preventing one or more symptoms of inflammatory or auto-immune diseases in a subject including applying to or implanting into the body of a patient one or more compositions including SAP are also provided. The patient can suffer from a primary, secondary, or acquired metabolic disorder, such as diabetes. In some embodiments, the administration of compositions of SAP also enhances the healing of one or more damaged or diseased tissues. For example, in some embodiments, the methods decrease the amount of time required for the damages or diseased tissue to heal by, for example, at least about 10%, at least about 30%, or at least about 50%, relative to an untreated control, as well as reduce inflammation.

In some embodiments, the self-assembled structure acts as a barrier to the passage of fluid. The self-assembled structure is optically clear and prevents the contamination and/or infection of diseased or damaged tissue(s) to which it is applied.

### BRIEF DESCRIPTION OF THE DRAWINGS

FIG. 1 is a schematic depicting the migration and morphological changes of microglial cells upon activation. The morphological changes that accompany microglial cell activation are characterized as each of 12 different stages (1A-6A and 6R-1R, respectively). This is used as a marker of inflammation.

FIGS. 2A-2H are line graphs showing the cytokine release profiles of THP-1 cells (arbitrary units on y axis), in the presence of 0 μg, 1 μg, 10 μg, and 100 μg of (RADA)4 in the absence of LPS (first set of “0, 1, 10, 100” on y axis) and presence of LPS (second set of “0, 1, 10, 100” on y axis), for each of complement component C5a (FIG. 2A), TNFSF2 (FIG. 2B), IL-6 (FIG. 2C), IL-8/CXCL8 (FIG. 2D), MCP-1/CCL2 (FIG. 2E), MIP-1/CCL3 (FIG. 2F), MIP-1/CCL4 (FIG. 2G), and IL-1/IL1-F2 (FIG. 2H), respectively.

FIGS. 3A-3B are bar graphs showing the levels of inflammation in the livers of pigs (FIG. 3A; n=2 for each condition) or rats (FIG. 3B; n=3-4 for each condition) at the indicated time points post application of the EARA-16 (SEQ ID NO: 89) and RADA-16 (SEQ ID NO: 1) SAP. Values are normalized to the saline control.

FIG. 4 is a bar graph showing the levels of inflammation in the kidneys of rats treated with EARA-16 (SEQ ID NO: 89) or KS (Ac-(RADA)₃CVSVPQAL-CONH₂; SEQ ID NO: 413) at the indicated time points. Inflammation was assayed thorough Ed-1 reactivity (IHC staining). Values are normalized to the saline (uninjured kidney) control which is set to 100%.

FIG. 5 is a bar graph showing the levels of inflammation in the olfactory bulb after damage and administration of saline as a control, or one of RADA-16 (SEQ ID NO: 1) or EARA-16 (SEQ ID NO: 89) SAP solutions. Inflammation was assayed using GFAP to reactive astrocytes. Values are normalized to the saline control at day 2. N=7 for each condition.

### DETAILED DESCRIPTION OF THE INVENTION

### I. Definitions

The term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%.

“Biocompatible” refers to compatibility with living tissue or a living system by not being toxic, injurious, or physiologically reactive and not causing immunological rejection. Biocompatible materials, along with any metabolites or degradation products thereof, are generally non-toxic to the recipient and do not cause any significant adverse effects to the recipient. Biocompatible materials are generally materials which do not elicit a significant inflammatory or immune response when administered.

“Biodegradable” generally refers to a material that under physiologic conditions degrades or erodes to smaller units or chemical species that are capable of being metabolized, eliminated, or excreted by the subject. The degradation time is a function of composition and morphology and can last, for instance, hours, weeks or months. In some embodiments, degradation can include disassembly of SAP structures.

“Complementary” means having the capability of forming ionic or hydrogen bonding interactions between hydrophilic residues in adjacent peptides in a structure. Hydrophilic residues in a peptide contain either hydrogen bonds or ionically pair with a hydrophilic residue on an adjacent peptide, or is exposed to solvent. In most cases, the peptides assemble hydrophobic to hydrophobic and hydrophilic to hydrophilic, although hydrophobic to hydrophilic can occur. The structure of the molecule will change during assembly over time. Alignment can and does change during packing. In the case of an SAP such as RADA (SEQ ID NO: 57), the hydrophobic face will assemble while the hydrophilic face will assemble in aqueous solvent; in the case of an oil solvent, the peptide will assemble hydrophilic-hydrophilic, with the hydrophobic face oriented into the oil. Pairing may also involve van der Waals forces.

“Effective amount” refers to the amount necessary to elicit a desired response, which may vary depending on such factors as the desired outcome, the agent being delivered, the nature of the site, the nature of the conditions for which the agent is administered, etc. For example, the effective amount of a composition for treatment of a disease or disorder may be an amount sufficient to promote recovery to a greater extent than would occur in the absence of the composition.

“Preventing” refers to reducing the risk that a condition, state, disease, or symptom, or the manifestation or worsening thereof, will occur.

The terms “treat”, “treatment” and “treating” refer to the reduction or amelioration of the progression, severity and/or duration of one or more symptoms of an injury, disease or disorder, delay of the onset of a disease or disorder, or the amelioration of one or more consequences, indications or symptoms (preferably, one or more discernible symptoms) of an injury, disease or disorder, resulting from the administration of one or more therapies (e.g., one or more therapeutic agents such as a compound as described).

“Increase,” “enhance,” “stimulate,” “induce” and like terms generally refer to the act of directly or indirectly improving or increasing a function or behavior relative to the natural, expected, or average or relative to current conditions. For instance, something that increases, stimulates, induces or enhances anti-inflammatory effects might induce the production, and/or secretion of anti-inflammatory cytokines, and/or infiltration of immune cells that mediate anti-inflammatory responses, such as Treg or Th17 cells.

The term “self-assembling” refers to the spontaneous or induced assembly of molecules into defined, stable, non-covalently bonded structures that are held together by intermolecular and/or intramolecular forces.

“Small Molecule” refers to a molecule having a relatively low molecular weight, such as less than about 1000 or 1,500 g/mol. Typically, small molecules are not peptides or nucleic acids.

The term “carrier” or “excipient” refers to an organic or inorganic, natural or synthetic inactive ingredient in a formulation, with which one or more additional ingredients are combined. Typically, a carrier or an excipient is an inert substance added to a pharmaceutical composition to further facilitate its administration, does not interfere with its activity or properties, and/or does not cause significant irritation to the recipient.

The term “topical administration”, means the non-invasive administration to the skin, orifices, or mucosa. Topical administrations can be administered locally and can provide a local effect in the region of application without systemic exposure. Topical formulations can provide systemic effect via adsorption into the blood stream of the individual. Topical administration can include, but is not limited to, cutaneous, transdermal, intravesicular, or mucosal (rectal, buccal, intranasal, or intravaginal, and rectal) administration.

### II. Compositions

It has been established that SAP are useful for reducing and/or preventing one or more symptoms of immune processes that constitute inflammation. In some embodiments, the SAP compositions are in an amount and concentration effective to reduce or prevent one or more symptoms of inflammation, such as pain, irritation, swelling, redness or other discoloration, loss of sensation, reduced mobility, fever, headache, itching, discharge of pus, headache, chills, muscle stiffness, immobility of a joint, loss of function of an organ, stimulation of nerve endings by bradykinin, increased blood flow, malaise, and physiological responses associated with production of histamine and/or heparin associated with undesirable inflammation.

In some embodiments, the compositions are formulated for topical application directly onto inflamed tissue. In other embodiments, the compositions are formulated for administration into one or more internal structures, for example, by injection or implantation. The compositions can be partially biodegradable, completely biodegradable, or non-biodegradable. Because the SAP form a self-assembled matrix structure capable of surrounding or containing a prosthetic implant or tissue graft, they are particularly useful for preventing or reducing the inflammatory processes associated with graft rejection.

In some embodiments, compositions for administration to inflamed tissue include non-assembled, precursor SAP. In other embodiments, compositions for administration into inflamed tissue include self-assembled structures formed from assembly of the precursor peptides or peptidomimetics thereof. In other embodiments, compositions for administration into the body include combinations of assembled and non-assembled precursor peptides or peptidomimetics thereof. Therefore, the SAP can be assembled prior to administration or after the composition has been administered.

Typically, the assembly of the SAP is initiated upon contact with physiological fluids. Therefore, in some embodiments, compositions of substantially non-assembled SAP are induced to assemble in vivo upon administration to the body (e.g., upon contact with blood, pus, or other bodily fluids). In other embodiments, compositions of substantially non-assembled SAP are induced to assemble in vitro (e.g., by contacting the SAP with an ionic solution to initiate assembly), prior to administration to the body.

Typically, the SAP assemble to form a continuous peptide structure upon contacting physiological fluids at the site of administration. In some embodiments, the structure acts as a barrier to the passage of bodily fluids and/or contaminants. In some embodiments, where assembly is initiated upon contact with physiological fluids upon contacting the body, the compositions form a fluid-impermeable, self-assembled barrier structure that coats the surface of the inflamed tissue. The barrier structure prevents the movement of substances such as bodily fluids, cells and contaminants through the structure. At the site of administration, the assembled SAP form a continuous surface and reduces undesirable evaporation of fluids from diseased or damaged tissue, whilst providing a barrier to infectious agents and contaminants.

In some embodiments, the SAP compositions can optionally include one or more additional agents, such as tissue-binding motifs, tissue-recognition motifs, therapeutic agents, diagnostic agents, cells, and combinations thereof. The one or more additional agents may not be bound to the SAP, or may be covalently or non-covalently bound to the SAP.

A. SAPs

Compositions for reducing or preventing one or more symptoms of inflammation include SAP, amino acid residues or peptidomimetics that are capable of self-assembly, or combinations thereof. In some embodiments, SAP compositions include mixtures of self-assembling peptides and self-assembling peptidomimetics. In other embodiments, self-assembling peptides include combinations of standard amino acids and non-standard amino acids.

1. SAP

The term “peptide” includes “polypeptide,” “oligopeptide,” and “protein,” and refers to a chain of at least two α-amino acid residues linked together by covalent bonds (peptide bonds). “Peptide” may refer to an individual peptide or to a collection of peptides having the same or different sequences, any of which may contain naturally occurring α-amino acid residues, non-naturally occurring α-amino acid residues, and combinations thereof. In particular, the D-enantiomer (“D-α-amino acid”) of residues may be used. When D-α-amino acid residues (Xaa) are included within a sequence, they are annotated as “Xaa^{D}”. α-Amino acid analogs are also known in the art and may be employed. Suitable non-naturally occurring amino acids include, but are not limited to, D-alloisoleucine(2R,3S)-2-amino-3-methylpentanoic acid, L-cyclopentyl glycine (S)-2-amino-2-cyclopentyl acetic acid.

Peptides can be represented as amino acid residue sequences. Those sequences are written left to right in the direction from the amino (“N-”) to the carboxyl (“-C”) terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). A “variant” of a peptide refers to a polypeptide that differs from a reference polypeptide but retains essential properties. A variant and reference polypeptide may differ in amino acid sequence by one or more modifications (e.g., substitutions, additions, and/or deletions of one or more residues) relative to the reference peptide.

Modifications and changes (e.g., a conservative amino acid substitution) can be made in the structure of the polypeptides without substantially affecting the self-assembly characteristics of the polypeptide. For example, certain amino acids can be substituted for other amino acids in a sequence without appreciable variation in activity. In making such changes, the hydropathic index of amino acids can be considered. It is known that certain amino acids can be substituted for other amino acids having a similar hydropathic index or score and still result in a polypeptide with similar functional activity. It is known in the art that an amino acid can be substituted by another amino acid having a similar hydropathic index and still obtain a functionally equivalent polypeptide.

Substitution of like amino acids can also be made on the basis of charge. In certain embodiments, the substitution of amino acids having an equivalent charge under physiological conditions can be made in the structure of the polypeptides of the disclosure without substantially affecting the self-assembly characteristics of the polypeptide. Charge states negative (“−ve”), positive (“+ve”), and non-charged or neutral (“neu”) can be assigned to amino acid residues under physiological conditions as follows: aspartate (−ve); glutamate (−ve); arginine (+ve); lysine (+ve); histidine (neu or +ve); serine (neu); asparagine (neu); glutamine (neu); glycine (neu); proline (neu); threonine (neu); alanine (neu); cysteine (neu); methionine (neu); valine (neu); leucine (neu); isoleucine (neu); tyrosine (neu); phenylalanine (neu); tryptophan (neu).

Useful peptides can vary in length so long as they retain the ability to self-assemble to an extent useful for one or more of the purposes. The number of amino acid residues in the peptide may range from as few as four α-amino acid residues to as many as 100 residues. Typically, peptides which self-assemble have from about 4 to about 64 residues, more preferably from about 8 to about 36 residues, most preferably from about 8 to about 16 residues. In preferred embodiments, the peptide has from about 8 to about 12 residues, or about 12 to about 16 residues, or about 16 to about 20 residues. In yet another embodiment, the peptide has from about 16 to about 24 residues, or from about 16 to about 28 residues, or from about 16 to about 32 residues.

One or more of the amino acid residues in a SAP can be altered or derivatized by the addition of one or more chemical entities including, but not limited to, acyl groups, carbohydrate groups, carbohydrate chains, phosphate groups, farnesyl groups, isofarnesyl groups, fatty acid groups, or a linker which allows for conjugation or functionalization of the peptide. For example, either or both ends of a given peptide can be modified. The carboxyl and/or amino groups of the carboxyl- and amino-terminal residues, respectively can be protected or not protected. The charge at a terminus can also be modified. For example, a group or radical such as an acyl group (RCO—, where R is an organic group (e.g., an acetyl group (CH₃CO—)) can be present at the N-terminus of a peptide to neutralize an “extra” positive charge that may otherwise be present (e.g., a charge not resulting from the side chain of the N-terminal amino acid). Similarly, a group such as an amine group (RNH—, where R is an organic group (e.g., an amino group —NH₂)) can be used to neutralize an “extra” negative charge that may otherwise be present at the C-terminus (e.g., a charge not resulting from the side chain of the C-terminal amino acid residue). Where an amine is used, the C-terminus bears an amide (—CONHR). The neutralization of charges on a terminus may facilitate self-assembly. One of ordinary skill in the art will be able to select other suitable groups.

Useful peptides can also be branched, in which case they will contain at least two peptide “branches”, each of which includes at least three amino acid residues joined by peptide bonds. The two peptide branches may be linked by a bond other than a peptide bond.

The peptides can have an amphiphilic nature (e.g., the peptides can contain approximately equal numbers of hydrophobic and hydrophilic amino acid residues), which can be complementary and structurally compatible. Complementary peptides have the ability to form ionic or hydrogen bonds with residues on adjacent peptides in a structure. For example, one or more hydrophilic residues in a peptide can either hydrogen bond or ionically pair with one or more hydrophilic residues on an adjacent peptide. Hydrophilic residues typically contain a polar functional group or a functional group that is charged at physiological conditions. Exemplary functional groups include, but are not limited to, carboxylic acid groups, amino groups, sulfate groups, hydroxyl groups, halogen groups, nitro groups, phosphate groups, etc. Hydrophobic residues are those residues that contain non-polar functional groups. Exemplary functional groups include, but are not limited to, alkyl groups, alkene groups, alkyne groups, and phenyl groups.

In one embodiment, the hydrophilic residue has the formula —NH—CH(X)—COO—, wherein X has the formula (CH₂)_{y}Z, wherein y=0-8, preferably 1-6, more preferably 1-4, and most preferably 1-3, and Z is a polar or charged functional group including, but not limited to, a carboxylic acid group, an amino group, a sulfate group, a hydroxyl group, a halogen group, a nitro group, a phosphate group, or a functional group containing a quaternary amine. The alkyl chain can be in a linear, branched, or cyclic arrangement. X may also contain one or more heteroatoms within the alkyl chain and/or X may be substituted with one or more additional substituents. In a preferred embodiment, Z is a carboxylic acid group or an amino group. In one embodiment, the hydrophobic residue has the formula —NH—CH(X)—COO—, wherein X has the formula (CH₂)_{y}Z, wherein y=0-8, preferably 1-6, more preferably 1-4, and more preferably 1-3, and Z is a non-polar functional group including, but not limited to, an alkyl group, an alkene group, an alkyne group, or a phenyl group. The alkyl, alkene, or alkyne chain can be in a linear, branched, or cyclic arrangement. X may also contain one or more heteroatoms within the alkyl chain and/or X may be substituted with one or more additional substituents. In a preferred embodiment, X is an alkyl group, such as a methyl group.

In one embodiment, the SAP includes peptides having a sequence of amino acid residues conforming to one or more of Formulas I-IV:

((Xaa^{neu}−Xaa⁺)ₓ(Xaa^{neu}−Xaa⁻)_{y})ₙ (I)

((Xaa^{neu}−Xaa⁻)ₓ(Xaa^{neu}−Xaa⁺)_{y})ₙ (II)

((Xaa⁺−Xaa^{neu})ₓ(Xaa⁻−Xaa^{neu})_{y})ₙ (III)

((Xaa⁻−Xaa^{neu})ₓ(Xaa⁺−Xaa^{neu})_{y})ₙ (IV)

wherein each Xaa^{neu} represents an amino acid residue having a neutral charge; Xaa⁺ represents an amino acid residue having a positive charge; Xaa⁻ represents an amino acid residue having a negative charge; x and y are integers having a value of 1, 2, 3, or 4, independently; and n is an integer having a value of 1-5.

Useful peptides can also include one or more amino acid residues having a neutral charge between one or more sets of residues conforming to any one of Formulas I-IV. For example, in some embodiments, peptides include Formulas III and IV, linked with a single amino acid residue having a neutral charge, or linked with two amino acid residues having a neutral charge, or linked with three amino acid residues having a neutral charge.

Peptides with modulus I (i.e., peptides having alternate positively and negatively charged R groups on one side (e.g., the polar face of the 0-sheet) are described by each of Formulas I-IV, where x and y are 1. Examples of peptides of modulus I include, but are not limited to, RADA (SEQ. ID NO. 57) and RADARADARADARADA (SEQ. ID NO. 1). Examples of peptides of modulus II (i.e., peptides having two residues bearing one type of charge (e.g., a positive charge) followed by two residues bearing another type of charge (e.g., a neutral charge)) are described by the same formulas where both x and y are 2. Examples of peptides of modulus III (i.e., peptides having three residues bearing one type of charge (e.g., a positive charge) followed by three residues bearing another type of charge (e.g., a negative charge)) include, but are not limited to, RARARADADADA (SEQ. ID NO. 414). Examples of peptides of modulus IV (i.e., peptides having three residues bearing one type of charge (e.g., a positive charge) followed by three residues bearing another type of charge (e.g., a negative charge)) include, but are not limited to, RARARARADADADADA (SEQ. ID NO. 415).

In some embodiments, the SAP includes peptides having a sequence of amino acid residues including one or more of Formulas V-XII:

Xaa^{neu}((Xaa^{neu}−Xaa⁺)ₓ(Xaa^{neu}−Xaa⁻)_{y})ₙ; (V)

Xaa^{neu}((Xaa^{neu}−Xaa⁻)ₓ(Xaa^{neu}−Xaa⁺)_{y})ₙ; (VI)

((Xaa⁺−Xaa^{neu})ₓ(Xaa⁻−Xaa^{neu})_{y})ₙXaa^{neu}; (VII)

((Xaa⁻−Xaa^{neu})ₓ(Xaa⁺−Xaa^{neu})_{y})ₙXaa^{neu}; (VIII)

((Xaa^{neu}−Xaa⁺)ₓ(Xaa^{neu}−Xaa⁻)_{y})ₙXaa^{neu}; (IX)

((Xaa^{neu}−Xaa⁻)ₓ(Xaa^{neu}−Xaa⁺)_{y})ₙXaa^{neu}; (X)

Xaa^{neu}((Xaa⁺−Xaa^{neu})ₓ(Xaa⁻−Xaa^{neu})_{y})ₙ; (XI)

Xaa^{neu}((Xaa⁻−Xaa^{neu})ₓ(Xaa⁺−Xaa^{neu})_{y})ₙ; (XII)

Wherein each Xaa^{neu} represents an amino acid residue having a neutral charge; Xaa⁺ represents an amino acid residue having a positive charge; Xaa⁻ represents an amino acid residue having a negative charge; x and y and z are integers having a value of 1, 2, 3, or 4, independently; and n is an integer having a value of 1-5.

Where SAP are used, it is thought that their side chains (or R groups) partition into two faces, a polar face with positively and/or negatively charged ionic side chains (e.g., side chains containing —OH, —NH, —CO₂H, or —SH groups), and a nonpolar face with side chains that are considered neutral or uncharged at physiological pH (e.g., the side chain of an alanine residue or residues having other hydrophobic groups). The positively charged and negatively charged amino acid residues on the polar face of one peptide can form complementary ionic pairs with oppositely charged residues of another peptide. These peptides may therefore be called ionic, self-complementary peptides. If the ionic residues alternate with one positively and one negatively charged residue on the polar face (−+−+−+−+), the peptides may be described as “modulus I;” if the ionic residues alternate with two positively and two negatively charged residues (−−++−−++) on the polar face, the peptides are described as “modulus II;” if the ionic residues alternate with three positively and three negatively charged residues (++++−−−+++−−−) on the polar face, the peptides are describe as “modulus III;” if the ionic residues alternate with four positively and four negatively charged residues (++++−−−−++++−−−−) on the polar face, they are described as “modulus IV.” A peptide having four repeating units of the sequence EAKA (SEQ ID NO: 77) may be designated EAKA16-I (SEQ ID NO: 76), and peptides having other sequences may be described by the same convention.

Other hydrophilic residues that form hydrogen bonds including, but not limited to, asparagine and glutamine, may be incorporated into the peptides. If the alanine residues in the peptides are changed to more hydrophobic residues, such as leucine, isoleucine, phenylalanine or tyrosine, the resulting peptides have a greater tendency to self-assemble and form peptide matrices with enhanced strength. Some peptides that have similar amino acids sequences and lengths as the peptides form alpha-helices and random-coils, rather than beta-sheets, and do not form macroscopic structures. In addition to self-complementarity, other factors are likely to be important for the formation of macroscopic structures, such as the peptide length, the degree of intermolecular interaction, and the ability to form staggered arrays.

Unpaired residues can interact (e.g., form hydrogen bonds, etc.) with the solvent. Peptide-peptide interactions may also involve van der Waals forces and/or forces that do not constitute covalent bonds. The peptides are structurally compatible when they are capable of maintaining a sufficiently constant intrapeptide distance to allow self-assembly and structure formation. The intrapeptide distance can vary. The term “intrapeptide distance” refers to the average of a representative number of distances between adjacent amino acid residues. In one embodiment, the intrapeptide distance is less than about 4 angstroms, preferably less than about 3 angstroms, more preferably less than about 2 angstroms, and most preferably less than about 1 angstrom. The intrapeptide distance may be larger than this, however. These distances can be calculated based on molecular modeling or based on a simplified procedure described in U.S. Pat. No. 5,670,483 to Zhang, et al.

The compositions including SAP can be formed through self-assembly of the peptides described in U.S. Pat. Nos. 5,670,483; 5,955,343; 6,548,630; 6,800,481; 7,098,028; 9,327,010; and U.S. Pat. No. 9,364,513 to Zhang, et al.; U.S. Pat. Nos. 9,162,005; 9,415,084; and U.S. Pat. No. 9,339,476 to Ellis-Behnke, et al.; Holmes, et al., Proc. Natl. Acad. Sci. USA, 97:6728-6733 (2000); Zhang, et al., Proc. Natl. Acad. Sci. USA, 90:3334-3338 (1993); Zhang, et al., Biomaterials, 16:1385-1393 (1995); Caplan et al., Biomaterials, 23:219-227 (2002); Leon, et al., J. Biomater. Sci. Polym. Ed., 9:297-312 (1998); and Caplan, et al., Biomacromolecules, 1:627-631 (2000). See also WO 2007/142757.

In some embodiments, SAP include one or more segments of positively or negatively charged residues. For example, these segments can include a sequence of positively or negatively charged residues, for example, about 2 to about 50 amino acid residues, typically about 3 to about 30 residues, more typically about 10 to about 20 amino acid residues. In some embodiments, about half of the residues of an SAP can be positively charged and about half of the residues can be negatively charged. For example, an SAP can have the following sequence RRRRDDDD (SEQ ID NO: 416) or GGGGSSSS (SEQ ID NO: 417). A combination of these peptides can self-assemble by matching or aligning the positive end of a first SAP to the negative end of a second SAP. The SAP can stack-up or aggregate.

In some embodiments, an SAP can contain a segment of residues that have either a positive or negative charge under physiological conditions. For example, representative amino acid sequences for positively charged SAP include, but are not limited to, KKKK (SEQ ID NO: 418), RRRR (SEQ ID NO: 419), or HHHH (SEQ ID NO: 420). Representative amino acid sequences for negatively charged SAP include, but are not limited to, DDDD (SEQ ID NO: 421) or EEEE (SEQ ID NO: 422). When combined, a string of positively charged amino acid residues will align parallel and opposite with a string of negatively charged amino acid residues. In certain embodiments, strings of positively charged amino acids will alternate with strings of negatively charged amino acids to for a multilayered structure.

In some embodiments, an SAP can contain sequences in which at least one hydrophobic residue alternates with at least one hydrophilic residue (under physiological conditions). For example, the sequence of a representative SAP can be GQGQ (SEQ ID NO: 423), GGQQGG (SEQ ID NO: 424), GQQGQQG (SEQ ID NO: 425), GGQGGQGG (SEQ ID NO: 426), etc.

The partitioning of the SAP into a polar or non-polar environment can be controlled by altering the hydrophobic to hydrophilic amino acid residue ratio, wherein a ratio greater than 1:1 indicates that the peptide partitions more in hydrophobic conditions while a ratio of less than 1:1 indicates that the peptide partitions more in hydrophilic conditions.

The compositions, regardless of the precise form (e.g., whether in a liquid form or molded) and regardless of the overall compositions (e.g., whether combined with another agent, contained within a device, or packaged in a kit), can include a mixture of one or more peptides. Peptide-based structures can be formed of heterogeneous mixtures of peptides (i.e., mixtures containing more than one type of peptide conforming to a given formula or to two or more of the formulas). In some embodiments, each of the types of peptides in the mixture can self-assemble with the same type of peptide. In other embodiments, one or more of each type of peptide would not self-assemble alone, but the combination of heterogeneous peptides may self-assemble (i.e., peptides in the mixture are complementary and structurally compatible with each other). Thus, either a homogeneous mixture of self-complementary and self-compatible peptides of the same sequence or containing the same repeating subunit, or a heterogeneous mixture of different peptides, which are complementary and structurally compatible to each other, can be used.

In some embodiments, mixtures of one or more peptide sequences produce structures having combined properties of the different sequences used. The physical properties of self-assembled peptide structures vary according to the ratio of the different SAP from which they are formed.

In some embodiments, SAP structures include two or more layers of structurally distinct SAP structures, for example, formed by consecutive administration and assembly of each peptide onto the surface of the other. Therefore, in some embodiments, the structural and biochemical properties of each surface of a multi-layered SAP structure are different, according to the different properties of the SAP from which they are formed, respectively.

One or more short amino acid sequences that assists in self-assembly (referred to as assembly assist sequences) can be added to a homogeneous or heterogeneous mixture of amino acid sequences that alone do not self-assemble. The assembly assist sequences contain amino acids that are complementary with the amino acids in the sequences in the mixture. The assembly assist sequences may contain any number of amino acids. Preferably, the assembly assist sequences contain at least four amino acids. The assembly assist sequences may contain a flexible linker between the amino acids that assists in self-assembly. For example, the assembly assist sequence may contain a pair, a triad, or a quartet of assembly assisting amino acids at the termini of the sequence which are connected via a flexible linker. Suitable assembly assist sequences include, but are not limited to, RADA (SEQ ID NO: 57) and EAKA (SEQ ID NO: 77).

Suitable linkers include, but are not limited to, ether based tethers such as polyethylene glycol (PEG), N-succinimidyl 3-(2-pyridyldithio)propionate (SPDP, 3- and 7-atom spacer), long-chain-SPDP (12-atom spacer), (succinimidyloxycarbonyl-α-methyl-2-(2-pyridyldithio) toluene) (SMPT, 8-atom spacer), succinimidyl-4-(N-maleimidomethyl)cyclohexane-1-carboxylate) (SMCC, 11-atom spacer) and sulfosuccinimidyl-4-(N-maleimidomethyl)cyclohexane-1-carboxylate, (sulfo-SMCC, 11-atom spacer), m-maleimidobenzoyl-N-hydroxysuccinimide ester (MBS, 9-atom spacer), N-(γ-maleimidobutyryloxy) succinimide ester (GMBS, 8-atom spacer), N-(γ-maleimidobutyryloxy) sulfosuccinimide ester (sulfo-GMBS, 8-atom spacer), succinimidyl 6-((iodoacetyl) amino) hexanoate (SIAX, 9-atom spacer), succinimidyl 6-(6-(((4-iodoacetyl)amino)hexanoyl)amino)hexanoate (SIAXX, 16-atom spacer), and p-nitrophenyl iodoacetate (NPIA, 2-atom spacer). One of ordinary skill in the art also will recognize that a number of other linkers with varying numbers of atoms may be used.

SAP structures can be formed that have varying degrees of stiffness or elasticity. The structures typically have a low elastic modulus (e.g., a modulus in the range of between about 0.01 and about 1,000 kPa, preferably between about 1 and about 100 kPa, more preferably between about 1 and about 10 kPa as measured by standard methods, such as in a standard cone-plate rheometer). Low values may be preferable, as they permit structure deformation as a result of movement, in response to pressure, for instance in the event of cell contraction. Stiffness can be controlled in a variety of ways, including by changing the length, sequence, and/or concentration of the precursor molecules (e.g., SAP). Other methods for increasing stiffness can also be employed. For example, one can attach to the precursors either biotin or other molecules that can be subsequently cross-linked or otherwise bonded to one another. The molecules (e.g., biotin) can be included at an N- or C-terminus of a peptide/peptidomimetic or attached to one or more residues between the termini. Where biotin is used, cross-linking can be achieved by subsequent addition of avidin. Other cross-linkable molecules can be used, for example, amino acid residues with polymerizable groups such as vinyl groups may be incorporated and cross-linked by exposure to UV light. The extent of crosslinking can be precisely controlled by applying the radiation for a predetermined length of time. The extent of crosslinking can be determined by light scattering, gel filtration, scanning electron microscopy, or other methods well known in the art. Crosslinking can be assessed by HPLC or mass spectrometry analysis of the structure after digestion with a protease, such as a matrix metalloprotease. Material strength may be determined before and/or after cross-linking. Regardless of whether cross-linking is achieved by a chemical agent or light energy, the molecules may be cross-linked in the course of creating a mold or when peptide-containing solutions are applied to tissue.

SAP chains can be cross-linked (e.g., to form a spider web-type pattern) to reinforce the material in vivo. The crosslinks can serve to reinforce the material to provide increased rigidity and strength. For example, an SAP functionalized with a polymerizable group at the periphery can be applied to the surface of inflamed tissue. Upon crosslinking, the peripheral material becomes more rigid, anchoring the material to the surface of the tissue or other surrounding tissue while the interior material remains flexible to move with the body.

Factors influencing the physical properties of self-assembled peptide structures within or associated with inflamed tissue include, but are not limited to, peptide sequence, peptide length, presence of bound agents, presence of tissue-specific or tissue-binding motifs, for example, a tissue-specific peptide sequence, as well as the amount of peptide (e.g., concentration, mass and volume), peptide form (e.g., powder or solution) and assembly-state at application time.

The half-life (e.g., the in vivo half-life) of the structures can also be modulated by incorporating protease or peptidase cleavage sites into the precursors that subsequently form a given structure. Proteases or peptidases that occur naturally in vivo or that are administered can promote degradation by cleaving their cognate substrates.

Combinations of any of the modifications here can be made. For example, SAP that include a protease cleavage site and a cysteine residue and/or a cross-linking agent, kits and devices containing them, and methods of using them, can be utilized.

The peptide structures formed from any SAP made by any process can be characterized using various biophysical and optical techniques, such as circular dichroism (CD), dynamic light scattering, Fourier transform infrared (FTIR), atomic force (tension) microscopy (ATM), scanning electron microscopy (SEM), and transmission electron microscopy (TEM). For example, biophysical methods can be used to determine the degree of beta-sheet secondary structure in the peptide structure. Filament and pore size, fiber diameter, length, elasticity, and volume fraction can be determined using quantitative image analysis of scanning and/or transmission electron micrographs. The structures can also be examined using several standard mechanical testing techniques to measure the extent of swelling, the effect of pH and ion concentration on structure formation, the level of hydration under various conditions, the tensile strength, as well as the manner in which various characteristics change over the period of time required for the structures to form and degrade.

Typically, the SAP are biocompatible, non-toxic, fully or partially biodegradable, and do not cause local or systemic inflammation. Preferably, breakdown products of the SAP do not cause secondary toxicity and are preferably suitable for growth and repair of the surrounding tissues.

2. Peptidomimetics

Another class of materials that can self-assemble is peptidomimetics. The term “peptidomimetics”, refers to non-natural peptide molecules, which mimic peptide structure. Peptidomimetics typically retain the ability to produce the same biological effect as a parent peptide and can interact with the biological target of the parent peptide. Peptidomimetics may be used to circumvent some of the problems associated with a natural peptide: e.g. stability against proteolysis and poor bioavailability (Vagner J., et al. Curr. Opin. Chem. Biol., 12(3): 292-296 (2008)). Self-assembling peptidomimetics are molecules that are structurally similar to peptides having a segment of residues having a positive charge under physiological conditions joined to a segment of residues having a negative charge under physiological conditions.

Peptidomimetics have general features analogous to peptides, such as amphiphilicity. Examples of peptidomimetics are described in Moore et al., Chem. Rev. 101(12), 3893-4012 (2001), and in WO 2007/142757.

The peptidomimetics can be classified into four categories: α-peptides, β-peptides, γ-peptides, and δ-peptides. Peptides including combinations of more than one of α-amino acids, β-amino acids, γ-amino acids, and α-amino acids can also be used. For example, SAP can include alpha-amino and beta-amino acid residues (i.e., alpha-beta peptides), alpha-amino and delta-amino acid residues (i.e., alpha-delta peptides), and alpha-amino and gamma-amino acid residues (i.e., alpha-gamma peptides).

The alpha amino acids can be classical or non-classical alpha amino acids (i.e., L-form or D-form, or combinations thereof). Examples of α-peptide peptidomimetics that can be used include, but are not limited to, N,N′-linked oligoureas, oligopyrrolinones, oxazolidin-2-ones, azatides and azapeptides.

Examples of β-peptides include, but are not limited to, β-peptide foldamers, β-aminoxy acids, sulfur-containing β-peptide analogues, and hydrazino peptides.

Examples of γ-peptides include, but are not limited to, γ-peptide foldamers, oligoureas, oligocarbamates, and phosphodiesters.

Examples of δ-peptides include, but are not limited to, alkene-based δ-amino acids and carbopeptoids, such as pyranose-based carbopeptoids and furanose-based carbopeptoids.

SAP can be generated, for example, which differ from those exemplified by a single amino acid residue or by multiple amino acid residues (e.g., by inclusion or exclusion of a repeating quartet). For example, one or more cysteine residues may be incorporated into the peptides, and these residues may bond with one another through the formation of disulfide bonds. Structures bonded in this manner may have increased mechanical strength relative to structures made with comparable peptides that do not include cysteine residues and thus are unable to form disulfide bonds.

3. Exemplary SAP

In an exemplary embodiment, a self-assembling peptidomimetic includes both alpha amino acids (annotated as Xaa) and beta amino acids (annotated as Xaa^{B}). Exemplary self-assembling peptidomimetic sequences include EA^{B}KA^{B}EA^{B}KA^{B}EA^{B}KA^{B}EA^{B}KA^{B} (SEQ ID NO: 427); EA^{B}KA^{B}EA^{B}KA^{B} (SEQ ID NO: 428); RA^{B}DA^{B}RA^{B}DA^{B}RA^{B}DA^{B}RA^{B}DA^{B} (SEQ ID NO: 429); and RA^{B}DA^{B}RA^{B}DA^{B} (SEQ ID NO: 430).

Examples of representative hydrophobic and hydrophilic SAP sequences are listed in Table 1.

TABLE 1
Representative SAP

| | SEQ ID |
|---|---|
| Sequence (N → C) | NO: |
| RADARADARADARADA | 1 |
| SGSGSGSGSGSGSGSG | 2 |
| SASASASASASASASA | 3 |
| SVSVSVSVSVSVSVSV | 4 |
| SLSLSLSLSLSLSLSL | 5 |
| SISISISISISISISI | 6 |
| SMSMSMSMSMSMSMSM | 7 |
| SFSFSFSFSFSFSFSF | 8 |
| SWSWSWSWSWSWSWSW | 9 |
| SPSPSPSPSPSPSPSP | 10 |
| TGTGTGTGTGTGTGTG | 11 |
| TATATATATATATATA | 12 |
| TVTVTVTVTVTVTVTV | 13 |
| TLTLTLTLTLTLTLTL | 14 |
| TITITITITITITITI | 15 |
| TMTMTMTMTMTMTMTM | 16 |
| TFTFTFTFTFTFTFTF | 17 |
| TWTWTWTWTWTWTWTW | 18 |
| TPTPTPTPTPTPTPTP | 19 |
| CGCGCGCGCGCGCGCG | 20 |
| CACACACACACACACA | 21 |
| CVCVCVCVCVCVCVCV | 22 |
| CLCLCLCLCLCLCLCL | 23 |
| CICICICICICICICI | 24 |
| CMCMCMCMCMCMCMCM | 25 |
| CFCFCFCFCFCFCFCF | 26 |
| CWCWCWCWCWCWCWC | 27 |
| CPCPCPCPCPCPCPCP | 28 |
| YGYGYGYGYGYGYGYG | 29 |
| YAYAYAYAYAYAYAYA | 30 |
| YVYVYVYVYVYVYVYV | 31 |
| YLYLYLYLYLYLYLYL | 32 |
| YIYIYIYIYIYIYIYI | 33 |
| YMYMYMYMYMYMYMYM | 34 |
| YFYFYFYFYFYFYFYF | 35 |
| YWYWYWYWYWYWYWYW | 36 |
| YPYPYPYPYPYPYPYP | 37 |
| NGNGNGNGNGNGNGNG | 38 |
| NANANANANANANANA | 39 |
| NVNVNVNVNVNVNVNV | 40 |
| NLNLNLNLNLNLNLNL | 41 |
| NINININININININI | 42 |
| NMNMNMNMNMNMNMNM | 43 |
| NFNFNFNFNFNFNFNF | 44 |
| NWNWNWNWNWNWNWNW | 45 |
| NPNPNPNPNPNPNPNP | 46 |
| QGQGQGQGQGQGQGQG | 47 |
| QAQAQAQAQAQAQAQA | 48 |
| QVQVQVQVQVQVQVQV | 49 |
| QLQLQLOLOLOLOLOL | 50 |
| QIQIQIQIQIQIQIQI | 51 |
| QMQMQMQMQMQMQMQM | 52 |
| QFQFQFQFQFQFQFQF | 53 |
| QWQWQWQWQWQWQWQW | 54 |
| QPQPQPQPQPQPQPQP | 55 |
| AEAKAEAKAEAKAEAK | 56 |
| RADA | 57 |
| RAEARAEARAEARAEA | 58 |
| KADAKADAKADAKADA | 59 |
| ARADARADARADA | 60 |
| RADARADARADARADARADA | 61 |
| ARADARADARADARADARADA | 62 |
| ARADARADARADARADA | 63 |
| RLDLRLDLRLDLRLDL | 64 |
| RLDL | 65 |
| RLDLRL | 66 |
| RADARA | 67 |
| LRLDLR | 68 |
| IEIKIEIKIEIKI | 69 |
| IEIKIEIKIEIKIEIK | 70 |
| IEIKIEIKIEIKIEIKI | 71 |
| IEIKIEIKIEIKIEIKIEIK | 72 |
| IEIKIEIKIEIKIEIKIEIKI | 73 |
| IEIKIEIKIEIK | 74 |
| EIKIEIKIEIKIEIKI | 75 |
| EAKAEAKAEAKAEAKA | 76 |
| EAKA | 77 |
| EAKAEAKAEA | 78 |
| EAKAEAKAEAKAEAKAEAKA | 79 |
| AEAKAEAKAEAKAEAKA | 80 |
| AEAKAEAKAEAKA | 81 |
| RADARADARADARADLRA | 82 |
| RDADDDADRDADDDADRDADDDADRDADDDAD | 83 |
| RDADDDADRDADDDADRDADDDAD | 84 |
| EDADKDADEDADKDADEDADKDAD | 85 |
| EDADKDAD | 86 |
| RDADDDAD | 87 |
| RADARADA | 88 |
| EARAEARAEARAEARA | 89 |
| EARAEARAEARA | 90 |
| EARAEARAE | 91 |
| EARA | 92 |

B. Tissue-Specific Components

The SAP may contain a tissue-specific component (“TSC”), which can be peptides, polysaccharides, or glycoproteins that are present within the body, or that are specific to the tissue surrounding or in contact with inflamed tissue. For example, a TSC may bind to a single cell type or to cells found in one type of tissue, in connective tissue, to epithelial cells, or by species (human) cells, or specific organs or organelles.

In some embodiments, a TSC selectively binds to tissues that express or exhibit one or more markers of inflammation or the inflammatory response. Exemplary markers include, but are not limited to, cytokines, and/or the presence or absence of immune effector cells. Exemplary immune effector cells include macrophage cells, such as microglial cells in the brain and central nervous system, alveolar cells in the lungs, Kupffer cells in the liver, histocytes in connective tissue, mesangial cells in the kidney, osteoclasts in the bones and Langerhans cells in the skin.

The TSC can target cell specific surface carbohydrates. For example, cell surface carbohydrates are major components of the outer surface of mammalian cells and are very often characteristic of cell types. Cell type-specific carbohydrates are involved in cell-cell interactions. In some embodiments, the TSC is a sequence of amino acids that recognizes and interacts with one or more components of injured or diseased tissue. In some embodiments, the TSC interacts with a ligand or component that is common to many tissues, and is also expressed at the site of inflammation. In other embodiments, the TSC interacts with a sequence that is not present or exposed in healthy tissue. In certain embodiments, the TSC interacts with one or more of the components of the extracellular matrix (ECM). The SAP can be modified such that they can anchor or interact with the structural ECM at the edges of blood vessels and/or tissues. ECM is any material part of a tissue that is not part of any cell, and is the defining feature of connective tissue. The ECM's main components are various glycoproteins, proteoglycans and hyaluronic acid. In most animals, the most abundant glycoproteins in the ECM are collagens. ECM also contains many other components: proteins such as fibrin, elastin, fibronectins, laminins, and nidogens, and minerals such as hydroxylapatite, or fluids such as blood plasma or serum with secreted free flowing antigens. In addition, the ECM sequesters a wide range of cellular growth factors, and acts as a local depot for them. Changes in physiological conditions can trigger protease activities that cause local release of such depots. This allows the rapid and local activation of cellular functions, without de novo synthesis. Given this diversity, ECM can serve many functions, such as providing support and anchorage for cells, providing a way of separating the tissues, and regulating intercellular communication.

1. TSC Sequences

Peptides or proteins can be used in combination with or alternation with the SAP. In some embodiments, the TSC is conjugated to the SAP (e.g., at the N-terminus and/or C-terminus). An exemplary motif that can serves as a TSC is the sequence MSCRAMM (SEQ ID NO: 141). Representative TSC are provided in Table 2.

TABLE 2
Tissue-specific Components

| Sequence (N → C) | SEQ ID NO: |
|---|---|
| Pmp(Y(Me)ITNCP-Orn-Y)NH₂ | 409 |
| Mpr(YFQNCPR) | 410 |
| (CYFQNCPRG)NH₂ | 411 |
| CYFQNCPR | 412 |
| (CYIQNCPRG)NH₂ | 93 |
| (YFQN(Asu)PRG)NH₂ | 94 |
| (YIQN(Asu)PRG)NH₂ | 95 |
| Mpr-D-PyridylAnine | 96 |
| (FQNCPRG)NH₂ | |
| (Deamino-Pen-YFVNCPDRG)NH₂ | 97 |
| Mpr(YFQNCPRG)NH₂ | 98 |
| Mpr(YFQNCPDRG)NH₂ | 99 |
| Mpr(YFQNCPK) | 100 |
| (CYFQNCPKG)NH₂ | 101 |
| CYFQNCPK | 102 |
| Mpr(YFVNCPDRG)NH₂ | 103 |
| (CFIQNCP-Orn-G)NH₂ | 104 |
| Pmp(DY(OEt)FVNCP-Cit-G)NH₂ | 105 |
| Pmp(Y(OEt)FVNCPRG)NH₂ | 106 |
| Pmp(Y(Me)FQNCPRG)NH₂ | 107 |
| Pmp(Y(Me)IQNCP-Orn-G)NH₂ | 108 |
| GDRGDSP | 109 |
| GDRGDSPASSK | 110 |
| G-Pen-GRGDSPCA | 111 |
| GRADSP | 112 |
| GRGDDSP | 113 |
| GRGDNP | 114 |
| GRGDS | 115 |
| GRGDSP | 116 |
| GRGDSPC | 117 |
| GRGDSPK | 118 |
| GRGDTP | 119 |
| GRGES | 120 |
| GRGESP | 121 |
| GRGETP | 122 |
| KGDS | 123 |
| GAVSTA | 124 |
| WTVPTA | 125 |
| TDVNGDGRHDL | 126 |
| REDV | 127 |
| RGDC | 128 |
| RGDS | 129 |
| RGDSPASSKP | 130 |
| RGDT | 131 |
| RGDV | 132 |
| RGES | 133 |
| SDGR | 134 |
| SDGRG | 135 |
| YRGDS | 136 |
| EGVNDNEEGFFSAR | 137 |
| YADSGEGDFLAEGGGVR | 138 |
| G(Glp)VNDNEEGFFSARY | 139 |
| GPR | N/A |
| CVSVPQAL | 140 |
| MSCRAMM | 141 |

Pmp = pyridoxamine phosphate
Mpr = 3-mercaptopropionyl
Deamino-Pen = deamino penicillamine
Pen = penicillamine
Asu = amino succinyl
OEt = ethoxy
Me = methyl
Cit = citrulline

C. Hydrophobic Peptide Sequences

Hydrophobic or hydrophilic tails can be added to the SAP. The tails can interact with cell membranes, thus anchoring the SAP on to the cell surface. Table 3 shows a list of peptides with hydrophobic tails.

TABLE 3
SAP including Hydrophobic Tails

| Sequence (N → C) | SEQ ID NO: |
|---|---|
| GGGGGDGDGDGDGDGD | 142 |
| GGGGGEGEGEGEGEGE | 143 |
| GGGGGKGKGKGKGKGK | 144 |
| GGGGGRGRGRGRGRGR | 145 |
| GGGGGHGHGHGHGHGH | 146 |
| AAAAADADADADADAD | 147 |
| AAAAAEAEAEAEAEAE | 148 |
| AAAAAKAKAKAKAKAK | 149 |
| AAAAARARARARARAR | 150 |
| AAAAAHAHAHAHAHAH | 151 |
| VVVVVDVDVDVDVDVD | 152 |
| VVVVVEVEVEVEVEVE | 153 |
| VVVVVKVKVKVKVKVK | 154 |
| VVVVVRVRVRVRVRVR | 155 |
| VVVVVHVHVHVHVHVH | 156 |
| LLLLLDLDLDLDLDLD | 157 |
| LLLLLELELELELELE | 158 |
| LLLLLKLKLKLKLKLK | 159 |
| LLLLLRLRLRLRLRLR | 160 |
| LLLLLHLHLHLHLHLH | 161 |
| IIIIIDIDIDIDIDID | 162 |
| IIIIIEIEIEIEIEIE | 163 |
| IIIIIKIKIKIKIKIK | 164 |
| IIIIIRIRIRIRIRIR | 165 |
| IIIIIHIHIHIHIHIH | 166 |
| MMMMMDMDMDMDMDMD | 167 |
| MMMMMEMEMEMEMEME | 168 |
| MMMMMKMKMKMKMKMK | 169 |
| MMMMMRMRMRMRMRMR | 170 |
| MMMMMHMHMHMHMHMH | 171 |
| FFFFFDFDFDFDFDFD | 172 |
| FFFFFEFEFEFEFEFE | 173 |
| FFFFFKFKFKFKFKFK | 174 |
| FFFFFRFRFRFRFRFR | 175 |
| FFFFFHFHFHFHFHFH | 176 |
| WWWWWDWDWDWDWDWD | 177 |
| WWWWWEWEWEWEWEWE | 178 |
| WWWWWKWKWKWKWKWK | 179 |
| WWWWWRWRWRWRWRWR | 180 |
| WWWWWHWHWHWHWHWH | 181 |
| PPPPPDPDPDPDPDPD | 182 |
| PPPPPEPEPEPEPEPE | 183 |
| PPPPPKPKPKPKPKPK | 184 |
| PPPPPRPRPRPRPRPR | 185 |
| PPPPPHPHPHPHPHPH | 186 |
| AAAAARADARADARAD | 187 |
| AAAAARARADADARAR | 188 |
| AAAAAEAKAEAKAEAK | 189 |
| AAAAAEAEAKAKAEAE | 190 |
| AAAAARAEARAEARAE | 191 |
| AAAAARARAEAEARAE | 192 |
| AAAAAKADAKADAKAD | 193 |
| AAAAAEAHAEAHAEAH | 194 |
| AAAAAEAEAHAHAEAE | 195 |
| AAAAARARARARADAD | 196 |
| AAAAARARARADADAD | 197 |
| AAAAAHADAHADAHAD | 198 |
| AAAAAHADADAHADAD | 199 |
| AAAAAHAEAEAHAEAE | 200 |
| GGGGGRGDGRGDGRGD | 201 |
| GGGGGRGRGDGDGRGR | 202 |
| GGGGGEGKGEGKGEGK | 203 |
| GGGGGEGEGKGKGEGE | 204 |
| GGGGGRGEGRGEGRGE | 205 |
| GGGGGRGRGEGEGRGE | 206 |
| GGGGGKGDGKGDGKGD | 207 |
| GGGGGEGHGEGHGEGH | 208 |
| GGGGGEGEGHGHGEGE | 209 |
| GGGGGRGRGRGRGDGD | 210 |
| GGGGGRGRGRGDGDGD | 211 |
| GGGGGHGDGHGDGHGD | 212 |
| GGGGGHGDGDGHGDGD | 213 |
| GGGGGHGEGEGHGEGE | 214 |
| VVVVVRVDVRVDVRVD | 215 |
| VVVVVRVRVDVDVRVR | 216 |
| VVVVVEVKVEVKVEVK | 217 |
| VVVVVEVEVKVKVEVE | 218 |
| VVVVVRVEVRVEVRVE | 219 |
| VVVVVRVRVEVEVRVE | 220 |
| VVVVVKVDVKVDVKVD | 221 |
| VVVVVEVHVEVHVEVH | 222 |
| VVVVVEVEVHVHVEVE | 223 |
| VVVVVRVRVRVRVDVD | 224 |
| VVVVVRVRVRVDVDVD | 225 |
| VVVVVHVDVHVDVHVD | 226 |
| VVVVVHVDVDVHVDVD | 227 |
| VVVVVHVEVEVHVEVE | 228 |
| LLLLLRLDLRLDLRLD | 229 |
| LLLLLRLRLDLDLRLR | 230 |
| LLLLLELKLELKLELK | 231 |
| LLLLLELELKLKLELE | 232 |
| LLLLLRLELRLELRLE | 233 |
| LLLLLRLRLELELRLE | 234 |
| LLLLLKLDLKLDLKLD | 235 |
| LLLLLELHLELHLELH | 236 |
| LLLLLELELHLHLELE | 237 |
| LLLLLRLRLRLRLDLD | 238 |
| LLLLLRLRLRLDLDLD | 239 |
| LLLLLHLDLHLDLHLD | 240 |
| LLLLLHLDLDLHLDLD | 241 |
| LLLLLHLELELHLELE | 242 |
| IIIIIRIDIRIDIRID | 243 |
| IIIIIRIRIDIDIRIR | 244 |
| IIIIIEIKIEIKIEIK | 245 |
| IIIIIEIEIKIKIEIE | 246 |
| IIIIIRIEIRIEIRIE | 247 |
| IIIIIRIRIEIEIRIE | 248 |
| IIIIIKIDIKIDIKID | 249 |
| IIIIIEIHIEIHIEIH | 250 |
| IIIIIEIEIHIHIEIE | 251 |
| IIIIIRIRIRIRIDID | 252 |
| IIIIIRIRIRIDIDID | 253 |
| IIIIIHIDIHIDIHID | 254 |
| IIIIIHIDIDIHIDID | 255 |
| IIIIIHIEIEIHIEIE | 256 |
| MMMMMRMDMRMDMRMD | 257 |
| MMMMMRMRMDMDMRMR | 258 |
| MMMMMEMKMEMKMEMK | 259 |
| MMMMMEMEMKMKMEME | 260 |
| MMMMMRMEMRMEMRME | 261 |
| MMMMMRMRMEMEMRME | 262 |
| MMMMMKMDMKMDMKMD | 263 |
| MMMMMEMHMEMHMEMH | 264 |
| MMMMMEMEMHMHMEME | 265 |
| MMMMMRMRMRMRMDMD | 266 |
| MMMMMRMRMRMDMDMD | 267 |
| MMMMMHMDMHMDMHMD | 268 |
| MMMMMHMDMDMHMDMD | 269 |
| MMMMMHMEMEMHMEME | 270 |
| FFFFFRFDFRFDFRFD | 271 |
| FFFFFRFRFDFDFRFR | 272 |
| FFFFFEFKFEFKFEFK | 273 |
| FFFFFEFEFKFKFEFE | 274 |
| FFFFFRFEFRFEFRFE | 275 |
| FFFFFRFRFEFEFRFE | 276 |
| FFFFFKFDFKFDFKFD | 277 |
| FFFFFEFHFEFHFEFH | 278 |
| FFFFFEFEFHFHFEFE | 279 |
| FFFFFRFRFRFRFDFD | 280 |
| FFFFFRFRFRFDFDFD | 281 |
| FFFFFHFDFHFDFHFD | 282 |
| FFFFFHFDFDFHFDFD | 283 |
| FFFFFHFEFEFHFEFE | 284 |
| WWWWWRWDWRWDWRWD | 285 |
| WWWWWRWRWDWDWRWR | 286 |
| WWWWWEWKWEWKWEWK | 287 |
| WWWWWEWEWKWKWEWE | 288 |
| WWWWWRWEWRWEWRWE | 289 |
| WWWWWRWRWEWEWRWE | 290 |
| WWWWWKWDWKWDWKWD | 291 |
| WWWWWEWHWEWHWEWH | 292 |
| WWWWWEWEWHWHWEWE | 293 |
| WWWWWRWRWRWRWDWD | 294 |
| WWWWWRWRWRWDWDWD | 295 |
| WWWWWHWDWHWDWHWD | 296 |
| WWWWWHWDWDWHWDWD | 297 |
| WWWWWHWEWEWHWEWE | 298 |
| PPPPPRPDPRPDPRPD | 299 |
| PPPPPRPRPDPDPRPR | 300 |
| PPPPPEPKPEPKPEPK | 301 |
| PPPPPEPEPKPKPEPE | 302 |
| PPPPPRPEPRPEPRPE | 303 |
| PPPPPRPRPEPEPRPE | 304 |
| PPPPPKPDPKPDPKPD | 305 |
| PPPPPEPHPEPHPEPH | 306 |
| PPPPPEPEPHPHPEPE | 307 |
| PPPPPRPRPRPRPDPD | 308 |
| PPPPPRPRPRPDPDPD | 309 |
| PPPPPHPDPHPDPHPD | 310 |
| PPPPPHPDPDPHPDPD | 311 |
| PPPPPHPEPEPHPEPE | 312 |
| SSSSSRSDSRSDSRSD | 313 |
| SSSSSRSRSDSDSRSR | 314 |
| SSSSSESKSESKSESK | 315 |
| SSSSSESESKSKSESE | 316 |
| SSSSSRSESRSESRSE | 317 |
| SSSSSRSRSESESRSE | 318 |
| SSSSSKSDSKSDSKSD | 319 |
| SSSSSESHSESHSESH | 320 |
| SSSSSESESHSHSESE | 321 |
| SSSSSRSRSRSRSRSR | 322 |
| SSSSSRSRSRSRSDSD | 323 |
| SSSSSRSRSRSDSDSD | 324 |
| SSSSSHSDSHSDSHSD | 325 |
| SSSSSHSHSHSHSHSH | 326 |
| SSSSSHSDSDSHSDSD | 327 |
| SSSSSHSESESHSESE | 328 |
| TTTTTRTDTRTDTRTD | 329 |
| TTTTTRTRTDTDTRTR | 330 |
| TTTTTETKTETKTETK | 331 |
| TTTTTETETKTKTETE | 332 |
| TTTTTRTETRTETRTE | 333 |
| TTTTTRTRTETETRTE | 334 |
| TTTTTKTDTKTDTKTD | 335 |
| TTTTTETHTETHTETH | 336 |
| TTTTTETETHTHTETE | 337 |
| TTTTTRTRTRTRTRTR | 338 |
| TTTTTRTRTRTRTDTD | 339 |
| TTTTTRTRTRTDTDTD | 340 |
| TTTTTHTDTHTDTHTD | 341 |
| TTTTTHTHTHTHTHTH | 342 |
| TTTTTHTDTDTHTDTD | 343 |
| TTTTTHTETETHTETE | 344 |
| CCCCCRCDCRCDCRCD | 345 |
| CCCCCRCRCDCDCRCR | 346 |
| CCCCCECKCECKCECK | 347 |
| CCCCCECECKCKCECE | 348 |
| CCCCCRCECRCECRCE | 349 |
| CCCCCRCRCECECRCE | 350 |
| CCCCCKCDCKCDCKCD | 351 |
| CCCCCECHCECHCECH | 352 |
| CCCCCECECHCHCECE | 353 |
| CCCCCRCRCRCRCRCR | 354 |
| CCCCCRCRCRCRCDCD | 355 |
| CCCCCRCRCRCDCDCD | 356 |
| CCCCCHCDCHCDCHCD | 357 |
| CCCCCHCHCHCHCHCH | 358 |
| CCCCCHCDCDCHCDCD | 359 |
| CCCCCHCECECHCECE | 360 |
| YYYYYRYDYRYDYRYD | 361 |
| YYYYYRYRYDYDYRYR | 362 |
| YYYYYEYKYEYKYEYK | 363 |
| YYYYYEYEYKYKYEYE | 364 |
| YYYYYRYEYRYEYRYE | 365 |
| YYYYYRYRYEYEYRYE | 366 |
| YYYYYKYDYKYDYKYD | 367 |
| YYYYYEYHYEYHYEYH | 368 |
| YYYYYEYEYHYHYEYE | 369 |
| YYYYYRYRYRYRYRYR | 370 |
| YYYYYRYRYRYRYDYD | 371 |
| YYYYYRYRYRYDYDYD | 372 |
| YYYYYHYDYHYDYHYD | 373 |
| YYYYYHYHYHYHYHYH | 374 |
| YYYYYHYDYDYHYDYD | 375 |
| YYYYYHYEYEYHYEYE | 376 |
| NNNNNRNDNRNDNRND | 377 |
| NNNNNRNRNDNDNRNR | 378 |
| NNNNNENKNENKNENK | 379 |
| NNNNNENENKNKNENE | 380 |
| NNNNNRNENRNENRNE | 381 |
| NNNNNRNRNENENRNE | 382 |
| NNNNNKNDNKNDNKND | 383 |
| NNNNNENHNENHNENH | 384 |
| NNNNNENENHNHNENE | 385 |
| NNNNNRNRNRNRNRNR | 386 |
| NNNNNRNRNRNRNDND | 387 |
| NNNNNRNRNRNDNDND | 388 |
| NNNNNHNDNHNDNHND | 389 |
| NNNNNHNHNHNHNHNH | 390 |
| NNNNNHNDNDNHNDND | 391 |
| NNNNNHNENENHNENE | 392 |
| QQQQQRQDQRQDQRQD | 393 |
| QQQQQRQRQDQDQRQR | 394 |
| QQQQQEQKQEQKQEQK | 395 |
| QQQQQEQEQKQKQEQE | 396 |
| QQQQQRQEQRQEQRQE | 397 |
| QQQQQRQRQEQEQRQE | 398 |
| QQQQQKQDQKQDQKQD | 399 |
| QQQQQEQHQEQHQEQH | 400 |
| QQQQQEQEQHQHQEQE | 401 |
| QQQQQRQRQRQRQRQR | 402 |
| QQQQQRORQRQRQDQD | 403 |
| QQQQQRQRQRQDQDQD | 404 |
| QQQQQHQDQHQDQHQD | 405 |
| QQQQQHQHQHQHQHQH | 406 |
| QQQQQHODQDQHQDQD | 407 |
| QQQQQHQEQEQHQEQE | 408 |

Hydrophilic tails can be added to the SAP, alone or in addition to hydrophobic tails, to facilitate interaction with the ECM of different vessels or tissues, such as the bladder.

D. Formulations of SAPs

The compositions can be used to prevent or limit movement of a bodily fluid, to stabilize tissue, components thereof or cells, or to prevent contamination when administered to a site in need thereof. The compositions can be in the form of a dry powder, a wafer, a disk, a tablet, a capsule, a liquid, a gel, a cream, a foam, an ointment, an emulsion, a coating on a medical device or implant (e.g., stent, catheter), incorporated into a microparticle, a polymeric matrix, a hydrogel, a fabric, a bandage, a suture, a tissue graft, or a sponge.

The concentration of the SAP in any given formulation can vary and can be between approximately 0.05% and 99%, inclusive, preferably between 0.1% and 10%. In one embodiment, the concentration of the SAP (e.g., in a liquid formulation) can be approximately 0.05-10.0% (0.5-100 mg/ml). The concentration of SAP can be higher in stock solutions and in solid (e.g., powdered) formulations. Solid preparations may have a concentration of SAP approaching 100% (e.g., the concentration of SAP can be 75, 80, 85, 90, 95, 96, 97, 98, 99% or more (e.g., 99.99%) of the composition).

In some embodiments, compositions of SAP are formulated for application to inflamed tissue as a dry powder. Dry powder formulations may contain at least 75% weight/weight (w/w) SAP, at least 80% w/w, at least 85% w/w, at least 90% w/w, at least 95% w/w, or more than 95% w/w.

In other embodiments, the SAP are formulated for application to a site of inflammation as a solution. SAP can be can be present in a solution that contains from about 0.25% weight:volume (w/v), to at least 7.5% w/v, preferably from about 1% w/v, to about 6% w/v, inclusive, for example, at least 0.1%, such as 0.1%-1%, 0.5%-5%, 1%-4%, 1%-5%, 1%-6%, 2%, 3%, 4% or 5% w/v. In some embodiments at least 75%, at least 80%, at least 85%, at least 90%, at least 95%, or more than 95% of the SAP have the same size and sequence. In particular embodiments, the SAP include two or more repeating units of the sequence RADA (SEQ ID NO: 57), two or more repeating units of the sequence EAKA (SEQ ID NO: 77), or combinations thereof.

In some embodiments, compositions of SAP formulated for application to inflamed tissue are preferably of low viscosity, and flow throughout the entire area to which they are applied. The concentration of SAP in the formulations for application to a site of inflammation can be between about 0.05% and about 0.5% weight to volume (wt/vol), for example, 0.1%-0.3% of the solution, in an amount sufficient to coat the entire surface or structure in need of treatment. Exemplary volumes for application of solutions of SAP to the body include an amount between about 10 μl and about 100 ml.

The viscosity of the SAP formulation may be adjusted to mimic that of a bodily substance, for example, synovial fluid. In other embodiments, the SAP forms a barrier to movement of fluids and bodily substances, for example, to prevent or reduce cell-cell interactions and cell-cell signaling within or at the surface of the tissue to which it is applied. In these embodiments, the concentration of the SAP within the solution is between about 1% and about 4% wt./vol., for example, 2.5% wt./vol.

Assembly of the SAP can be initiated or enhanced by the addition of an ionic solute or diluent to a solution of SAP or by a change in pH. For example, NaCl at a concentration of at least 5 mM can induce the assembly of macroscopic structures within a short period of time (e.g., within a few seconds to minutes). Lower concentrations of NaCl may also induce assembly but at a slower rate. Alternatively, self-assembly may be initiated or enhanced by introducing the SAP (whether dry, in a semi-solid gel, or dissolved in a liquid solution that is substantially free of ions) into a fluid (e.g., a physiological fluid such as blood or gastric juice) or an area (e.g., by topical application or by direct injection into tissue) including such ions. The gel does not have to be pre-formed prior to application to the desired site. Generally, self-assembly is expected to occur upon contacting the SAP with such a solution in any manner.

A wide variety of ions, including anions and cations (whether divalent, monovalent, or trivalent), can be used. For example, one can promote a phase transition by exposure to monovalent cations such as Li⁺, Na⁺, K⁺, Cs+ and Ca⁺. The concentration of such ions required to induce or enhance self-assembly is typically at least 5 nM to 5 mM. Lower concentrations also facilitate assembly, although at a reduced rate. When desired, SAP can be delivered with a hydrophobic material (e.g. pharmaceutically-acceptable oil) in a concentration that permits self-assembly, but at a reduced rate. When SAP are mixed with a hydrophobic agent such as an oil or lipid the assembly of the SAP forms different structures. In some cases when another material is added, the SAP will assemble into various other three dimensional structures that may be suitable for loading of a therapeutic agent. The hydrophilic part of the molecule will assemble in such a way as to minimize hydrophobic-hydrophilic interaction, thereby creating a barrier between the two environments. Several experiments have shown that the SAP aligns on the surface of the oil with the hydrophobic part of the molecule toward the surface and the hydrophilic portion of the molecule facing away from the oil, or will form toroidal-like structures with the hydrophobic material contained inside. This type of behavior enables the encapsulation of therapeutics or other molecules of interested for delivery in the body.

The composition may contain a salt scavenger to drive assembly to a preferred configuration. For example, circular dichroism (“CD”) experiments indicate that the assembly dynamics can be controlled using salt scavengers or salt enhancement to increase the formation of β-sheets, α-helices, or more random configurations. The compositions may optionally contain an indicator showing the configuration of the assembly (e.g., α-helix, β-sheet, lattice, etc.).

Alternatively, some of the described materials do not require ions to self-assemble but may self-assemble due to interactions with solvent, hydrophobic interactions, side chain interactions, and hydrogen bonding.

The materials can be formed within regularly or irregularly-shaped molds, which may include a surface of the body, or a cavity on the surface of the body, or a portion of a bodily structure (e.g., a tear in the skin, or de-epithelialized section of the skin) or which may be an inert material such as plastic or glass. The structures or scaffolds can be made to conform to a predetermined shape or to have a predetermined volume. To form a structure with a predetermined shape or volume (e.g., a desired geometry or dimension, including thin sheets or films), an aqueous solution of the SAP is placed in a pre-shaped casting mold, and the materials are induced to self-assemble by the addition of a plurality of ions. Alternately, the ions may be added to the solution shortly before placing the solution into the mold, provided that care is taken to place the solution into the mold before substantial assembly occurs. Where the mold is a tissue (whether in situ or not), the addition of an ionic solution may not be necessary. The resulting material characteristics, the time required for assembly, and the dimensions of the macroscopic structure that forms are governed by the concentration and amount of solution that is applied, the concentration of ions used to induce assembly of the structure, and the dimensions of the casting apparatus. The assembled material can achieve a gel-like or substantially solid form at room temperature, and heat may be applied to facilitate the molding (e.g., one can heat a solution used in the molding process (e.g., a precursor-containing solution) to a temperature ranging up to about body temperature (approximately 37° C.)). Once the assembled material has reached the desired degree of firmness, it can be removed from the mold and used for a described purpose. Alternatively, the materials described may be used to anchor host tissue to a tissue matrix or scaffold. For example, the described materials can be used as a “glue” to anchor host tissue that is to be regenerated to a tissue matrix or scaffold to ensure that the matrix or scaffold stays in place in the local environment to which it is injected or implanted. Tissue matrices and scaffolds are well known in the art and can be prepared from synthetic, semi-synthetic, and/or natural materials.

Materials that assemble and/or undergo a phase transition (e.g., a transition from a liquid state to a semi-solid, gel, etc.) when they come in contact with bodily fluids (e.g., the tear film) or an ionic solution are useful in providing an SAP structure at the surface of, or within one or more cavities of the body. The SAP structure is effective to reduce or prevent one or more symptoms of undesirable inflammation, or an inflammatory disease. It may be that the SAP reduce or prevent inflammation, reduce or prevent the formation of adhesions between diseased tissue and surrounding tissues, or induce and enhance the restoration of a damaged or diseased tissue.

Self-assembly or phase transition is triggered by components found in a subject's body (e.g., ions) or by physiological pH and is assisted by physiological temperatures. Self-assembly or phase transition can begin when the compositions are exposed to or brought into contact with a subject's body (e.g., at the surface of an organ such as the liver or kidney) and may be facilitated by the local application of heat to the area where the composition has been (or will be) deposited. Based on studies to date, self-assembly occurs rapidly upon contact with bodily fluids without the application of additional heat. The time required for effective assembly and/or phase transition can occur in 60 seconds or less (e.g., in 50, 40, 30, 20, or 10 seconds or less) following contact with a subject's tissue, or to conditions similar to those found within the body. For example, solutions containing SAP can form a self-assembled fluid-impermeable structure upon contact with physiological fluids within times as short as 10 seconds following application. In some circumstances, such as when conditions are sub-optimal (i.e., non-physiological), or when the concentration of self-assembling precursors is low, self-assembly or phase transition may take longer to achieve, for example, up to a minute, 5 minutes, 10 minutes, 30 minutes, an hour, or longer.

The compositions can form structures that are substantially rigid (e.g., solid or nearly solid) or that assume a definite shape and volume (e.g., structures that conform to the shape and volume of the location to which a liquid composition was administered, whether in vivo or ex vivo). The solidified SAP may be somewhat deformable or compressible after assembly or phase transition, but it will not substantially flow from one area to another, as compositions at a different point along the liquid to solid continuum may do, which may be due, at least in part, to their ability to undergo phase transitions. As a result, the compositions can also be used to prevent the movement of a bodily substance in a subject in need thereof. Self-assembly can be achieved in vivo or ex vivo by exposure to conditions within a certain range of physiological values, or non-physiological conditions. “Non-physiological conditions” refers to conditions within the body or at a particular site that deviate from normal physiological conditions at that site. Such conditions may result from trauma, surgery, injury, infection, or a disease, disorder, or condition. For example, a puncture wound in the stomach generally results in a decrease in the pH as stomach acid flows into the wound site. The SAP should self-assemble under such conditions. While liquid formulations are readily dispensed, the compositions administered may also be in a gel form that may become stiffer upon contact with physiological fluids at the site of application to the subject's body.

Regardless of the precise nature of the SAPs, upon exposure to conditions such as those described, the SAPs can form membranous two- or three-dimensional structures including a stable macroscopic porous matrix having ordered or non-ordered interwoven nanofibers (e.g., fibers approximately 5-20 nm in diameter, with a pore size of about 50-100 nm in a linear dimension). Three-dimensional macroscopic matrices can have dimensions large enough to be visible under low magnification (e.g., about 10-fold or less), and the membranous structures can be visible to the naked eye. Although three-dimensional, the structures can be exceedingly thin, including a limited number of layers of molecules (e.g., 2, 3, or more layers of molecules). Typically, each dimension of a given structure will be at least 10 μm in size (e.g., two dimensions of at least 100-1000 μm in size (e.g., 1-10 mm, 10-100 mm, or more)). The relevant dimensions may be expressed as length, width, depth, breadth, height, radius, diameter, or circumference in the case of structures that have a substantially regular shape (e.g., where the structure is a sphere, cylinder, cube, or the like) or an approximation of any of the foregoing where the structures do not have a regular shape.

The SAP can form a hydrated material when contacted with water under conditions such as those described (e.g., in the presence of a sufficient concentration (e.g., physiological concentrations) of ions (e.g., monovalent cations)). These may have a high water content (e.g., approximately 95% or more (e.g., approximately 96%, 97%, 98%, 99% or more)), and the compositions can be hydrated but not substantially self-assembled. A given value may be “approximate” in recognition of the fact that measurements can vary depending, for example, on the circumstances under which they are made and the skill of the person taking the measurement. Generally, a first value is approximately equal to a second when the first falls within 10% of the second (whether greater than or less than) unless it is otherwise clear from the context that a value is not approximate or where, for example, such value would exceed 100% of a possible value.

The properties and mechanical strength of the structures or scaffolds can be controlled as required through manipulation of the components therein. For example, the stiffness of an assembled gel can be increased by increasing the concentration of SAP therein. Alternatively, it may be desirable for different parts of the SAP structures to have different mechanical properties. For example, it may be advantageous to alter the stability or density of the SAP formulation by manipulating the amino acid sequence. This may be desirable when the SAP formulations are used to fill a void, such that the edges of the material self-assemble to attach to the tissue site while the rest of the SAP formulation flows out into the void. The sequences, characteristics, and properties of the SAP formulations and the structures formed by them upon self-assembly are discussed above.

E. Additional Agents

The compositions of SAP can include other agents, such as therapeutic, prophylactic or diagnostic agents. The additional agents are typically non-self-assembling. These can be a biomolecule which is a molecule such as a peptide, proteoglycan, lipid, carbohydrate, or a small molecule. Like small molecules, biomolecules can be naturally occurring or may be artificial (i.e., they may be molecules that have not been found in nature). For example, a protein having a sequence that has not been found in nature (e.g., one that does not occur in a publicly available database of sequences) or that has a known sequence modified in an unnatural way by a human hand (e.g., a sequence modified by altering a post-translational process such as glycosylation) is an artificial biomolecule. Nucleic acid molecules encoding such proteins (e.g., an oligonucleotide, optionally contained within an expression vector) are also biomolecules and can be incorporated into the compositions described. For example, a composition can include a plurality of SAP and cells that express, or that are engineered to express, a protein biomolecule (by virtue of containing a nucleic acid sequence that encodes the protein biomolecule).

Many different therapeutic, prophylactic or diagnostic agents can be incorporated into the formulation. One or more therapeutic, diagnostic and/or prophylactic agents can be administered simultaneously with the SAP in the same formulation, administered simultaneously in separate formulations, or sequentially. Alternatively, the agent(s) can be covalently or non-covalently coupled to the SAP, either directly or via an intermediate molecule. In some embodiments, compositions of SAP include one or more non-self-assembling therapeutic agents. In some embodiments, the compositions include one or more therapeutic agents, including, but not limited to, anti-angiogenesis agents, anti-infective agents, anti-inflammatory agents, analgesics, anesthetics, growth factors, immunosuppressant agents, anti-allergic agents, anti-oxidants, cytokines, and combinations thereof. For example, the additional agents can include one or more classes of therapeutic agents for the treatment of inflammatory or autoimmune diseases, such as anti-inflammatory agents, vasoactive agents, anti-infective agents, anesthetics, growth factors, vitamins or nutrients, and/or cells. Additional therapeutic agents can be selected according to the disease that is to be treated, and the route of administration. In some embodiments, additional therapeutic agents are suitable for topical application to the body. In other embodiments, additional therapeutic agents are suitable for parenteral application to the body.

In some embodiments, the therapeutic agent is present in its neutral form, or in the form of a pharmaceutically acceptable salt. In some cases, it may be desirable to prepare a formulation containing a salt of an agent due to one or more of the salt's advantageous physical properties, such as enhanced stability or a desirable solubility or dissolution profile. Generally, pharmaceutically acceptable salts can be prepared by reaction of the free acid or base forms of an agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, MD, 2000, p. 704.

In some embodiments, compositions of SAP include one or more non-self-assembling anti-inflammatory agents. The non-self-assembling anti-inflammatory agent can be non-steroidal, steroidal, or a combination thereof. One embodiment provides oral compositions containing about 1% (w/w) to about 5% (w/w), typically about 2.5% (w/w) of an anti-inflammatory agent. Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed.

Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

In some embodiments, compositions of SAP include one or more vasoconstrictor agents for local administration to inflamed tissue or surrounding tissues. Representative vasoconstrictors include epinephrine and phenylephrine. Vasoconstrictors such as phenylephrine can be included to prolong the effect of local anesthesia (e.g., 0.1-0.5% phenylephrine). Analgesic agents other than a local anesthetic agent, such as steroids, non-steroidal anti-inflammatory agents like indomethacin, platelet activating factor (PAF) inhibitors such as lexipafant, CV 3988, and/or PAF receptor inhibitors such as SRI 63-441 can be used.

In some embodiments, compositions of SAP include one or more local anesthetics for local administration to inflamed tissue or surrounding tissues. A local anesthetic is a substance that causes reversible local anesthesia and has the effect of loss of the sensation of pain. The SAP composition may include an anesthetic agent in an amount of, e.g., about 0.1%, about 0.2%, about 0.3%, about 0.4%, about 0.5%, about 0.6%, about 0.7%, about 0.8%, about 0.9%, about 1.0%, about 2.0%, about 3.0%, about 4.0%, about 5.0%, about 6.0%, about 7.0%, about 8.0%, about 9.0%, or about 10% by weight of the total composition. The concentration of local anesthetics in the compositions can be therapeutically effective meaning the concentration is adequate to provide a therapeutic benefit without inflicting harm to the patient.

In some embodiments, compositions of SAP include one or more anti-infective or antimicrobial agents (e.g., an antibiotic, antibacterial, antiviral, or antifungal agent) for local administration to inflamed tissue, or surrounding tissues.

Other therapeutic agents can be included in the compositions, such as growth factors to accelerate one or more aspects of the recovery from, or prevention of a disease or disorder (e.g., angiogenesis, cell migration, process extension, and cell proliferation). The growth factor or another agent can be a chemotactic substance, which has the ability, in vivo or in cell culture, to recruit cells to a site at which the substance is present. The cells recruited may have the potential to contribute to the formation of new tissue or to augment and reinforce existing, damaged tissue (e.g., by contributing structurally and/or functionally to the tissue (e.g., by providing growth factors or contributing to a desirable immune response)).

In some embodiments, the compositions include cells. Where cells are delivered to a patient (e.g., to treat or prevent one or more inflammatory or auto-immune diseases), autologous cells can be used.

F. Excipients, Carriers, and Devices

Compositions of SAP can include excipients suitable for administration onto or into the body. For example, compositions of SAP can be formulated into a composition suitable for topical administration onto the surface of inflamed tissue, or for injection into one or more of the internal bodily structures where inflammation is present, or the undesired symptom(s) is to be prevented. In some embodiments, formulations for application to the body are typically a liquid solution or suspension. These may be injected into the inflamed tissue, the tissue surrounding the inflamed tissue, or onto one or more exposed surfaces of an area where symptoms of inflammation are to be reduced or prevented. Topical administration can include application directly to exposed tissue, vasculature or to tissues or prostheses, for example, during surgery, or by direct administration to the skin.

In the preferred embodiment, the formulation is a liquid or reconstitutable powder, applied topically. The formulations can include a pharmaceutically acceptable carrier or are provided as part of a medical device or coating.

In some forms, the formulation is provided as a dry or lyophilized powder which can be administered directly as a powder which hydrates at the site of application.

Alternatively, the formulation is suspended or dissolved in a solvent, most preferably aqueous, and applied as a spray, paint, or injection. The formulation can also by administered in a hydrogel such as chitin, collagen, alginate, or a synthetic polymer. Any formulation suitable for application to the body (e.g., a liquid, which can be applied as a spray or a powder) can be used. In another embodiment, the formulation is provided as a coating on a device, for example a contact lens or adhesive bandage, which may be dissolved in an aqueous solution and dried on the device, or mixed with a polymeric carrier and applied to the device. In yet another embodiment, the formulation is provided in a bandage, foam or matrix, in which the peptides may be dispersed or absorbed.

The formulation can also be in the form of sutures, tape, or adhesive. In some embodiments, for example, where the formulation is administered to a patient that has a disease or disorder relating to a prior injury at the site of inflammation, the SAP are formulated either alone, or together with other agents (e.g., with anesthetics, anti-inflammatories, growth factors, anti-infectives, etc.), in the form of a foam, matrix or bandage, for example to reduce or stop suppuration, bleeding or loss of other bodily fluids, as required.

Suitable excipients can be selected based upon the desired assembly-state of the self-assembling precursor materials. For example, when the SAP are delivered as a solution, a suitable excipient may contain a concentration of ions below the threshold required to initiate assembly. Representative excipients include solvents, diluents, pH modifying agents, preservatives, antioxidants, suspending agents, wetting agents, viscosity modifiers, tonicity agents, stabilizing agents, and combinations thereof. A preferred excipient is water.

Solutions, suspensions, or emulsions for injection may be buffered, for example, with an effective amount of buffer necessary to maintain a pH suitable for administration to the intended site. Suitable buffers are well known by those skilled in the art and some examples of useful buffers are acetate, borate, carbonate, citrate, and phosphate buffers. For example, solutions, suspensions, or emulsions for intra-synovial administration may also contain one or more tonicity agents to adjust the isotonic range of the formulation. Suitable tonicity agents are well known in the art and include, for example, glycerin, mannitol, sorbitol, sodium chloride, and other electrolytes.

In some instances, the formulation is distributed or packaged in a liquid form. Alternatively, formulations for administration are packed as a solid, obtained, for example by lyophilization of a suitable liquid formulation. The solid can be reconstituted with an appropriate carrier or diluent prior to administration.

Typically, when the composition is dispensed in a multidose container that is to be used over a longer period of time, such as 24 hours, a preservative must be added to ensure microbiologic safety over the period of use.

Compositions of SAP can be formulated to include a pharmaceutically acceptable excipient for topical administration onto the surface of an inflamed tissue. They may contain suitable additives, such as preservatives, antioxidants, and stabilizing agents. In addition to direct administration to epidermal tissue, topical administration of SAP can be effective to reduce or prevent symptoms of inflammation if applied to the lungs, nasal, oral (sublingual, buccal), vaginal, or rectal mucosa.

Compositions can be delivered to the lungs and lung epithelial lining while inhaling when delivered either as an aerosol or spray dried particles having an aerodynamic diameter of less than about 5 microns.

A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.). Nektar, Alkermes and Mannkind all have inhalable insulin powder preparations approved or in clinical trials where the technology could be applied to the described SAP formulations.

Formulations for administration to the mucosa can typically be spray dried drug particles, which may be incorporated into a tablet, gel, capsule, suspension or emulsion. Standard pharmaceutical excipients are available from any formulator. Oral formulations may be in the form of chewing gum, gel strips, tablets or lozenges.

In some forms, compositions of SAP are formulated to include a pharmaceutically acceptable excipient for parenteral administration. For example, injectable solutions can be prepared by incorporating the SAP in the required amount in the appropriate solvent or dispersion medium with one or more pharmaceutically acceptable excipients, as required. Generally, dispersions are prepared by incorporating the various compositions into a sterile vehicle which contains the basic dispersion medium and the required other ingredients. In the case of powders for the preparation of injectable solutions, the preferred methods of preparation are vacuum-drying and freeze-drying techniques which yield a powder of the SAP plus any additional desired ingredient from a previously prepared solution thereof.

In some forms, pharmaceutical formulations for administration by injection are an aqueous solution or suspension of the SAPs. In preferred embodiments, formulations for injection into the body include less than 20 mM ions, for example, between 20 mM and 0.01 mM ions, inclusive. In a particular embodiment, formulations of SAP for injection into the body include a solution of the SAP in water. In other embodiments, the solutions of the SAP include one or more polymer conjugates. Exemplary solvents include, for example, water, Ringer's solution, phosphate buffered saline (PBS), and isotonic sodium chloride solution. The formulation may also be a sterile solution, suspension, or emulsion in a nontoxic, acceptable diluent or solvent such as 1,3-butanediol.

Solutions, suspensions, or emulsions for injection or instillation into the body may be buffered with an effective amount of buffer necessary to maintain a pH suitable for administration. Suitable buffers are well known by those skilled in the art. Non-limiting examples include acetate, borate, carbonate, citrate, and phosphate buffers.

Solutions, suspensions, or emulsions for injection or instillation administration may contain at least one tonicity agent to adjust the isotonic range of the formulation. Suitable tonicity agents are well known in the art. Examples include glycerin, mannitol, sorbitol, sodium chloride, and other electrolytes.

In some embodiments, SAP are formulated for injection into one or more cavities within a joint. In some embodiments, the SAP are formulated for intra-articular injection. Formulations for intra-articular injection can be formulated to a desired volume. For example, the volume suitable for intra-articular injection is typically an amount between about 0.01 and 20 ml, inclusive, for example, 0.5 ml, 1 ml, 2 ml, 3 ml, 5 ml, 10 ml or 15 ml. Preferred excipients for intra-articular injection are biocompatible, non-toxic and do not induce inflammation. The formulation for intra-articular injection can include an amount of SAP in solution sufficient to provide a viscosity equivalent to that of a physiological fluid, for example, synovial fluid. The injection volume and content is adjusted according to the site of application, the physiology of the joint, and in the disease or disorder for which treatment is sought. For example, in certain embodiments, the dose of SAP and volume for a single joint injection depends on amount of fluid in the joint, as well as the disease state (i.e., extent of bleeding, articular cartilage integrity, joint volume, and other variables).

Exemplary joints that can be injected with corresponding volumes of formulations of SAP include, but are not limited to, shoulder (e.g., 10 ml), elbow (e.g., 5 ml), wrist or thumb (e.g., 2 ml), fingers (e.g., 1 ml), hip (e.g., 5 ml), knee (e.g., 10 ml), ankle/foot (e.g., 5 ml), and toe (e.g., 1 ml). Solutions, suspensions, or emulsions for administration may also contain one or more preservatives to prevent bacterial contamination of the preparations. Suitable preservatives are known in the art, and include polyhexamethylene biguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

Solutions, suspensions, or emulsions for intra-articular administration may also contain one or more excipients known in the art, such as dispersing agents, wetting agents, and suspending agents.

The compositions of SAP can include additional organic and/or inorganic materials, for example, to provide a structure or physical support for the SAP. In some embodiments the additional materials provide structural support to the compositions, such as materials that provide a scaffold. Scaffold materials can be selected to provide physical strength, elasticity, porosity, solubility, volume and bulk, as required by the application. In certain embodiments, the scaffold material has mechanical and/or biological properties similar to the extracellular matrix (ECM).

Scaffold materials can include natural or synthetic polymers, including natural polymers such as polypeptides and proteins, which may create a scaffold onto which SAP, therapeutic agents, cells or other agents are attached or associated. In some embodiments, the described compositions include proteins, such as ECM proteins. Exemplary natural scaffold materials include alginate, fibrinogen, hyaluronic acid, starch, chitosan, silk, gelatin, dextran, elastin, collagen, and combinations thereof. In some embodiments, compositions of SAP include scaffold materials that are synthetic polymers. Exemplary synthetic polymers include poly(L-lactic acid co-ε-caprolactone) (PLCL); poly(DL-lactic acid) (PDLA) and poly(lactic-co-glycolicacid) (PLGA); poly(ethylene oxide) (PEO); poly(vinyl alcohol) (PVA); poly (methyl methacrylate) (PMMA); poly(ethylene-co-vinyl acetate) (PEVA); polystyrene; polyurethane; and mixtures thereof. In preferred embodiments the scaffold materials are biocompatible. In preferred embodiments the scaffold materials do not induce an immune response.

SAP structures can biodegrade at a time following application that is consistent with the time required for treatment, for example, the amount of time required for reduction or prevention of pain, swelling, redness, irritation, itching, discharge, headache, or fever, such as one day, one week, one month or more than one month following application.

Bandages including SAP are described. Bandages including SAP can be formulated for use in therapeutic or cosmetic applications, according to the needs of the intended recipient. In some embodiments, SAP are applied to commercially available wound bandages. Application of bandages to the site in need of reduced symptoms associated with inflammation can occur before or after assembly of the SAP has occurred, and can be carried out by any suitable means known in the art for application, such as spraying, coating, painting, etc.

In certain embodiments, compositions of SAP are applied in the form of a solution or powder directly onto gauze or other non-peptide structures. For example, SAP and/or scaffold materials can be contacted with tissue around and within the site of inflammation, and held in place by a bandage or gauze.

G. Kits

The SAP can be assembled in kits, together with instructions for use. The kit may also include one or more of a syringe (e.g., a barrel syringe or a bulb syringe), a needle, a pipette, gauze, sponges, or cotton, swabs, a bandage, a vascular patch, a disinfectant, surgical thread, scissors, a scalpel, a sterile fluid, a spray canister, including those in which a liquid solution is sprayed through a simple hand pump, a sterile container, disposable gloves or an eye dropper.

Exemplary kits include SAP, self-assembling peptidomimetics, or combinations of SAP and peptidomimetics, excipients, suitable means for application, and instruction for use. In some embodiments, kits include measured dosages of one or more SAP and/or self-assembling peptidomimetics for application at distinct times. For example, a kit can include one or more SAP having a different sequence, dose, or conjugated to a different or the same agent(s) for distinct applications to the same site. Dosages, and application regimens can be determined according to the needs of the patient, for example, as specified by a physician or within the instructions.

Kits can include one or more means for administration into or onto the body. Typically, the applicator will initiate or maintain contact between one or more SAP with one or more parts of the body. In some embodiments the applicator is pre-filled or loaded with the SAP. When SAP are contained within the applicator, the amount and formulation of the SAP formulation to be dispensed can be fixed or varied, for example, by the patient or physician. Exemplary, applicators include an eye-dropper, a syringe, a spray, an air-applicator, and a tube. In some embodiments, the SAP are provided in one or more containers, for example, in dried form (e.g., lyophilized), or a solution, tablet, wafer, or gel. In some embodiments the SAP are pre-packaged in a concentration stock powder or solution, with instructions for diluting to the desired concentration immediately prior to application.

Kits including dried SAP can be packaged with a desiccant. In some embodiments, kits include one or more pharmaceutically acceptable excipients for administration into or onto the body. The excipient can be contained within a separate container or within an applicator. Therefore, in some embodiments, an applicator can include one or more SAPs, and one or more excipients within the same or different compartments. In some embodiments, one or more SAP is diluted or otherwise mixed with one or more excipients within the applicator immediately prior to application. The amount and type of SAP to be administered can be pre-determined within the applicator, or varied according to the desired effects.

In some embodiments, kits include an air applicator for the directed application of SAP in the form of a powder to the surface of the body. The amount of SAP administered can be varied. The contacted surface area of the body can be adjusted, for example, to a narrowly-defined area, or the entire exposed portion of the body. In other embodiments, kits include apparatus for delivery of a powder into the body via a ballistic injection through the outer layers of the epidermis. In other embodiments, kits include an apparatus to deliver a solution or gel via a single or series of injections or infusion.

In some embodiments, kits include an applicator that will mix two or more different SAPs together, to be dispensed and/or delivered together in one application or in a series of applications. For example, the device can contain several chambers, each of which contains an SAP. The composition can be dispensed directly onto the site of administration, or can be mixed in a mixing chamber within the device prior to administration. In one embodiment, an applicator can be used to administer the same composition to the body on several different occasions, or different compositions to the body at the same or different times.

### III. Methods of Making SAP and Compositions Thereof

Compositions of SAP can be prepared using any techniques known in the art. SAP are typically synthesized using standard procedures, so any technique in the art suitable to prepare synthetic peptides can be used. All of the method steps can be performed in any suitable order unless otherwise indicated, or otherwise clearly contradicted by context.

A. Production of SAP

SAPcan be chemically synthesized or purified from natural or recombinantly-produced sources, by methods well known in the art. For example, peptides can be synthesized using standard F-moc chemistry.

Standard Fmoc (9-florenylmethoxycarbonyl) derivatives include Fmoc-Asp(OtBu)-OH, Fmoc-Arg(Pbf)-OH, and Fmoc-Ala-OH. Couplings are mediated with DIC (diisopropylcarbodiimide)/6-Cl-HOBT (6-chloro-1-hydroxybenzotriazole). In some embodiments, the last four residues of the peptide require one or more recoupling procedures. In particular, the final Fmoc-Arg(Pbf)-OH coupling can require recoupling. For example, a second or third recoupling can be carried out to complete the peptide using stronger activation chemistry such as DIC/HOAT (1-hydroxy-7-azabenzotriazole) or HATU (1-[bis(dimethylamino)methylene]-1H-1,2,3-triazolo[4,5-b]pyridinium 3-oxid hexafluorophosphate)/NMM (N-methylmorpholine).

Acidolytic cleavage of the peptide can be carried out with the use of carbocation scavengers (thioanisole, anisole and H₂O). Optimization can be achieved by varying the ratio of the components of the cleavage mixture. An exemplary cleavage mixture ratio is 90:2.5:2.5:5 (TFA-thioanisole-anisole-H₂O). The reaction can be carried out for 4 hours at room temperature.

In some embodiments the removal of residual impurities is carried out by wash steps. For example, trifluoroacetic acid (TFA) and organic impurities can be eliminated by precipitation and repeated washes with cold diethyl ether and methyl t-butyl ether (MTBE).

Peptides can be purified using high pressure liquid chromatography (HPLC). Suitable solvents for dissolving the peptides include neat TFA. In some embodiments, 8 mL TFA/g peptide is sufficient to fully dissolve peptides following precipitation. For example, TFA can be diluted into H₂O. Typically, the peptides remain soluble at TFA concentrations of 0.5% to 8% and can be loaded onto reverse phase (RP)-HPLC columns for salt exchange. Exemplary salt exchange methods use 3-4 column volumes of acidic buffer to wash away the TFA counter ion due to its stronger acidity coefficient. Buffers suitable for use in washing away the TFA counter ion include 0.1% HCl in H₂O.

Following removal of TFA, peptides can be eluted with a step gradient. Exemplary elution buffers include 30% acetonitrile (MeCN) vs. 0.1% HCl in H₂O. For acetate exchange, peptides can be loaded from the same diluted TFA solution, washed with 3-4 column volumes of 1% acetic acid (AcOH) in H₂O, followed by 2 column volumes of 0.1 M NH₄OAc in H₂O, pH 4.4. In some embodiments, the column is washed again with 3-4 column volumes of 1% AcOH in H₂O.

Peptides can be eluted from the columns using a step gradient of 30% MeCN vs. 1% AcOH in H₂O. In some embodiments the elution of peptides can be enhanced by acetate exchange. Exemplary buffers for acetate exchange include 0.1 M NH₄OAc in H₂O, pH 4.4.

Analytical HPLC can be carried out to assess the purity and homogeneity of peptides. An exemplary HPLC column for use in analytical HPLC is a PHENOMENEX® JUPITER® column. In some embodiments analytical HPLC is carried out using a column and buffer that are heated to a temperature that is greater than 25° C., for example 25-75° C. In a particular embodiment, analytical HPLC is carried out at temperatures of about 65° C. A step gradient can be used to separate the peptide composition. In some embodiments the gradient is from 1%-40% MeCN vs 0.05% TFA in H₂O. The change in gradient can be achieved over 20 min using a flow rate of 1 ml/min. Peptides can be detected using UV detection at 215 nm.

In some embodiments, methods of making the compositions for administration into the body include the step of sterilization. Where compositions are required to be sterilized or otherwise processed for the removal of undesirable contaminants and/or microorganisms, filtration is a preferred method. Filtration can be achieved using any system or procedures known in the art. In some embodiments, filtration removes contaminants or prevents the growth or presence of microorganisms. Exemplary microorganisms and contaminants that can be removed include bacteria, cells, protozoa, viruses, fungi, and combinations thereof. In some embodiments, the step of filtration is carried out to remove aggregated or oligomerized proteins. For example, solutions of self-assembling precursor peptides or peptidomimetics thereof can be filtered to remove assembled peptide structures or oligomers on the basis of size.

B. Fabrication of Compositions

When the SAP are used to form a gelled or solid structure, for example, to be applied to the body as part of a backing on a bandage or other support structure, the SAP can be produced using one or more techniques. Examples include templating onto the surface, injection molding of a formulation of SAP in a solvent, stamping of a dry powder or frozen formulation of SAP in a solvent, direct application of a slurry formulation containing SAP onto a stencil, or patterned surface, such as a bandage or adhesive bandage, or a composite structure formed by a combination of these methods.

In some embodiments, SAP formulations are dried or dehydrated to remove a solvent. Any methods known in the art can be used for the dehydration of compositions including SAP.

The term “dried” or “lyophilized” is used to describe the product of a process for the removal of the majority of the solvent from a material in solution, for example, by methods for dehydration, vacuum sublimation, or “lyophilization”. These methods typically remove a major portion of solvent, such as water, from the material, but can result in a residual amount of solvent within the “dry” product. For example, a lyophilized powder may include up to 10% w/w of water, for example, 5%, 3%, 2%, 1%, or 0.5% w/w of water.

Any composition or formulation of SAP can include up to 25% by mass of derivative molecules and/or degradation products. Exemplary derivative molecules and/or degradation products include, but are not limited to, amino acid substitutions, amino acid deletions, oligomers including dimer, trimmers, tetramers or higher-order oligomers, aggregates and impurities. In some embodiments, formulations of SAP include less than or equal to 20% derivative molecules and/or degradation products by mass, such as 15%, 10% or less than 10%, such as 5%, 1% or 0%.

### IV. Methods of Use

SAP can be used for the treatment of inflammation or inflammatory diseases, and/or the reduction or prevention of one or more symptoms of inflammation and/or inflammatory diseases. Methods of using SAP and compositions thereof for reducing or preventing one or more undesired symptoms of inflammation and/or inflammatory diseases are described. Symptoms of inflammation and/or inflammatory diseases that can be mitigated or prevented by the methods include pain, swelling, redness, irritation, itching, discharge of pus, headache, chills, muscle stiffness, immobility of a joint, loss of function of an organ, stimulation of nerve endings by histamine, stimulation of nerve endings by bradykinin, increased blood flow, fever, increased blood flow, malaise, and physiological responses associated with production of histamine and/or heparin.

Typically, the methods include administering the SAP directly to a site of undesired inflammation, or a site at risk of undesired inflammation. In an exemplary embodiment, the SAP are administered into or surrounding a joint in an amount effective to treat inflammation, inflammatory diseases or disorders, or one or more symptoms of diseases or disorders associated with inflammation or other undesired immune activity at or near the joint. For example, the methods can reduce or prevent one or more of pain, swelling, or stiffness associated with inflammation of a joint.

The SAP can be administered directly to a painful site, for example topically, or by injection or by instillation. In other embodiments, the SAP are administered into the bloodstream, for example, by intravenous injection, to prevent or reduce symptoms of systemic inflammatory processes at multiple locations distant from the point of administration.

Dosage units containing an amount of SAP to provide pain relief at or near a painful site resulting from inflammation are also provided.

SAP used in the compositions to treat inflammation or inflammatory diseases and/or to reduce or prevent one or more symptoms of inflammation and/or inflammatory diseases can be assembled prior to or at the time of application, either by contacting the composition with an ionic solution or allowing the composition to contact a bodily fluid.

All of the methods can include identification and selection of an individual in need of treatment, or identification of a site of inflammation. In preferred embodiments, SAP are administered once, twice, three times or more a day directly to the site of inflammation. The frequency will vary depending on the severity of symptoms. The formulation may be applied in the form of an emulsion or suspension, lotion, ointment, cream, gel, salve or powder and sustained or slow release, as well as lotion. The formulation may also be applied in a sprayable form.

In preferred embodiments, the dosage administered to the site of inflammation does not result in toxicity or other undesirable side effects at the site of administration, or elsewhere. For example, multiple applications per day for an extended period of time do not result in tissue damage or toxicity. The methods can reduce or prevent one or more symptoms of inflammation.

Methods for reducing or preventing pain and other symptoms of inflammatory responses associated with surgery and are also provided. In some embodiments, compositions of SAP are coated onto a medical device, a prosthetic devise or a tissue graft. An exemplary prosthetic or medical device that can be coated, treated or otherwise associated with self-assembling peptides or peptidomimetics is an exogenous tissue, for example, for use in transplant therapy. Exogenous tissue implants are implanted inside the body to replace or augment the recipient's own endogenous tissue, for example, an endogenous organ that is damaged, diseased or otherwise defective or removed. In some embodiments, coating, covering or otherwise associating a tissue implant with SAPs prior to, during or immediately following surgery to implant the tissue within a recipient, enhances the outcome of the transplant surgery. For example, in some embodiments, implanting an exogenous organ coated with SAP into a subject reduces or prevents graft-versus-host disease or host-versus-graft disease relative to implanting an untreated control tissue.

Methods of administering SAP into or onto a site of inflammation for treatment of inflammation or an inflammatory disease, reducing and preventing symptoms of inflammation or inflammatory diseases and disorders in a subject are described. In some embodiments, SAP can be administered into or onto the body prophylactically, to prevent symptoms of inflammation or inflammatory diseases and disorders in a subject identified at risk of injury, and/or at risk of inflammatory diseases or disorders. For example, in some embodiments, the methods reduce one or more of pain, irritation, swelling, redness or other discoloration, loss of sensation, reduced mobility, fever, headache, itching, discharge of pus, headache, chills, muscle stiffness, immobility of a joint, loss of function of an organ, stimulation of nerve endings by bradykinin, increased blood flow, malaise, and physiological responses associated with production of histamine and/or heparin in a subject who has recently undergone surgery, is about to undergo surgery, or in a subject during the course of surgery.

SAP can be administered via any means which is effective to deliver an effective amount of the SAP to the site of inflammation, or at risk of inflammation. For example, compositions can be administered by topical, intravenous, intramuscular, intra-articular, or intraperitoneal routes.

The SAP can be applied to the outer surface of the inflamed tissue, tissues contacting or surrounding the inflamed tissue, or into one or more inner bodily compartments to be treated. Exemplary tissues that can be contacted with the SAP include the skin, the mucosa, liver, lung, intestines, brain, stomach, muscle, bone, spleen, kidney, bladder, genitourinary system, heart, central nervous system, joints (including the intra-articular cavity, cartilage and tendon).

The SAP formulations can be administered using conventional techniques for administration to the intended tissue, including but not limited to, topical administration and via injection. The formulations of SAP can be injected using a syringe, or other suitable means for delivery.

In some embodiments, formulations of SAP are delivered directly to the site of inflammation or at risk of inflammation using mechanical delivery means. For administration, the SAP can be administered to a subject having a particular inflammatory condition or disorder. For example, SAP can be administered to a subject having been diagnosed with an inflammatory disease or disorder, or having one or more symptoms of an inflammatory disease or disorder. Typically, the subject is a human. Therefore, methods of using SAP can include one or more steps of identifying and/or selecting a subject in need of treatment.

In some embodiments, the subject is a patient that has one or more underlying medical conditions. Exemplary underlying medical conditions include metabolic diseases, autoimmune diseases, immuno-deficiencies, infectious diseases, cancer, and neurological diseases. In some embodiments, the subject is a patient that is undergoing therapy for an underlying medical condition. Therefore, in some embodiments, the subject can have one or more diseases or disorders that is associated with or caused by a therapeutic intervention. For prophylactic administration, the SAP can be administered to a subject at risk of developing symptoms associated with a particular inflammatory condition or disorder. Alternatively, prophylactic administration can be applied to avoid the onset of symptoms in a patient diagnosed with the underlying condition or disorder.

The SAP can be assembled before, during or after application to the body. For example, the SAP can be synthesized and the finished formulation exposed to an ionic solution to induce gel formation. The gelled structure can be stored until use. The gelled structure can be dehydrated prior to storage. The structure can be gelled in a mold to form a particular shape, for example, to fill or accommodate the area of the body to which it is applied. In other embodiments, the SAP precursors are synthesized and stored in a substantially non-assembled form. The non-assembled SAP precursors can be dried prior to storage. Immediately prior to use, the dehydrated or dried non-assembled SAP can be exposed to an ionic solution to initiate assembly. In still other embodiments, the gelled structure is applied or implanted in an unassembled form and the peptides assemble upon contact with a bodily fluid, such as blood. This can be useful, for instance, to allow the SAP to assemble and conform to the shape of the application site.

In some embodiments, methods include administering formulations of SAP including one or more therapeutic, prophylactic, and/or diagnostic agents, as discussed above. When the SAP are applied in the form of a gelled structure, the agent can be impregnated into a SAP structure and/or coated on the surface of the structure. In other embodiments, the methods include administering an agent that is covalently coupled to one or more of the SAPs. For example, in some embodiments, the formulations of SAP include a pH-adjusting agent which is released at the site of administration to alter the pH at the site of administration.

Methods for reducing or preventing the symptoms associated with inflammation can include the steps of identifying a subject and site in need of treatment, treating the site by administering SAP to or near the site, and monitoring the efficacy of treatment. In some embodiments, the steps of monitoring the efficacy of treatment includes repeating the administration.

Identification of a subject and site in need of treatment can include the step of identifying one or more markers of inflammation and/or autoimmune disorder at a site within a subject. In some embodiments, SAP include one or more ligands that selectively bind to tissues that express of exhibit one or more. Exemplary markers of inflammation or inflammatory responses include, but are not limited to cytokines, and/or the presence or absence of immune effector cells. Exemplary immune effector cells include macrophage cells, such as microglial cells in the brain and central nervous system, alveolar cells in the lungs, Kupffer cells in the liver, histocytes in connective tissue, mesangial cells in the kidney, osteoclasts in the bones and Langerhans cells in the skin. In some embodiments, the inflammatory state of a tissue is determined by assessing the activation state of immune effector cells. For example, in some embodiments, inflammatory status is determined based upon the different morphological stages of macrophage cells.

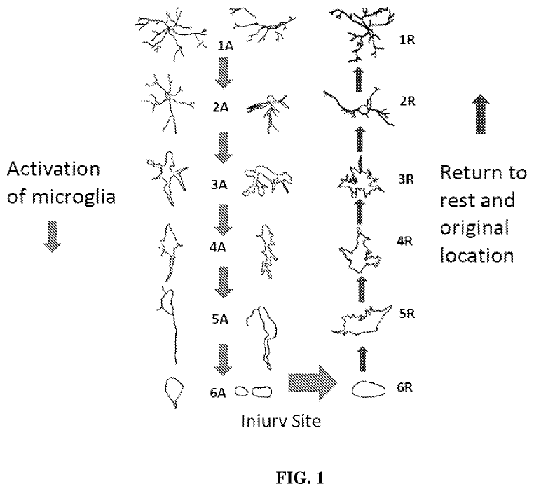

An exemplary method for assessing macrophage activation is via histological examination, according to the methods of Jonas, et al., PLoS One., 7(2):e30763 (2012), which is incorporated herein by reference in its entirety. Six stages of bidirectional microglial activation (A) and deactivation (R) have been observed (i.e., stage 1A to 6A). The cell body size increased, the cell process number decreased, and the cell processes retracted and thickened, orienting toward the direction of the injury site; until stage 6A, when all processes disappeared. In contrast, in deactivation stages 6R to 1R, the microglia returned to the original site exhibiting a stepwise retransformation to the original morphology. Thin highly branched processes re-formed in stage 1R, similar to those in stage 1A. This reverse transformation mirrored the forward transformation except in stages 6R to 1R: cells showed multiple nuclei which were slowly absorbed. A schematic representation of morphologically defined stepwise activation and deactivation of microglia cells is set forth in FIG. 1.

SAPs reduce the cytokine release associated with antigen-recognition by immune cells. It may be that the SAP assemble to form a physical barrier at the site of inflammation that mitigates the undesirable symptoms of inflammation. Therefore, SAP can be used for reducing or preventing one or more symptoms of inflammatory diseases (e.g., autoimmune diseases) characterized by undesirable activation of T-cells, cytokine release and corresponding biological functions of immune cells. For example, in some embodiments, SAPs reduce or prevent pain, irritation, swelling, redness or other discoloration, loss of sensation, reduced mobility, fever, headache, itching, discharge of pus, headache, chills, muscle stiffness, immobility of a joint, loss of function of an organ, stimulation of nerve endings by bradykinin, increased blood flow, malaise, and physiological responses associated with production of histamine and/or heparin resulting from diseases or disorders that are characterized by inflammation. Examples include allergy, asthma, autoimmune diseases, coeliac disease, glomerulonephritis, hepatitis, inflammatory bowel disease, reperfusion injury and transplant rejection.

Suppression of unwanted symptoms associated with inflammation resulting from disease and/or injury can be of high clinical importance in many environments. It has been demonstrated that SAP are capable of reducing the cytokine production of THP-1 cells (see Example 1). Therefore, SAP and compositions thereof, optionally including one or more additional therapeutic agents can be used to treat inflammation, an inflammatory disease or disorder, and/or to reduce or prevent one or more of the symptoms of inflammation or inflammatory diseases or disorders.

A. Autoimmune Diseases

In some embodiments, SAP and compositions thereof are used to reduce or prevent one or more symptoms associated with autoimmune diseases and disorders. In some embodiments, the autoimmune disease/disorder is rheumatoid arthritis (RA), systemic lupus erythematosus (SLE), Sjögren's syndrome, lupus nephritis, or multiple sclerosis.

Chronic and persistent inflammation is a major cause for the pathogenesis and progression of systemic autoimmune diseases such as rheumatoid arthritis (RA) and systemic lupus erythematosus (SLE). RA is a highly inflammatory polyarthritis often leading to joint destruction, deformity and loss of function. Additive, symmetric swelling of peripheral joints is the hallmark of the disease. Extra-articular features and systemic symptoms can commonly occur and may antedate the onset of joint symptoms. Chronic pain, disability and excess mortality are unfortunate sequelae. During progression of RA, the synovial lining layer of the inflamed joints increases its thickness as a result of synovial hyperplasia and infiltration into synovial stroma by CD4+ T cells, B cells, CD8+ T cells, macrophages, dendritic cells and neutrophils (Feldmann, et al., Cell, 85:307-10 (1996); Moreland, et al., N Engl J Med, 337:141-7 (1997)). In SLE, the production of autoantibodies results in the deposition of immune complex in many tissues and organs including glomeruli, skin, lungs and synovium, thereby generating rheumatic lesions with characteristic chronic inflammation and tissue damage.

B. Inflammatory Diseases

In some embodiments, SAP and compositions thereof are used to reduce or prevent one or more signs or symptoms of inflammatory responses within or around tissue affected by an inflammatory disease. Therefore, methods of administering SAP to one or more sites of inflammation for reducing and preventing symptoms of inflammatory diseases are provided. In some embodiments, the presence of SAP in or around tissue that is diseased or damaged by undesirable inflammation reduces or prevents the extent of damage, as compared to an untreated control.

Exemplary inflammatory diseases and disorders associated with undesirable symptoms of the inflammatory response include, but are not limited to, asthma, encephalitis, inflammatory bowel disease, chronic obstructive pulmonary disease (COPD), allergic disorders, septic shock, pulmonary fibrosis, undifferentiated spondyloarthropathy, undifferentiated arthropathy, arthritis, degenerative joint disease, inflammatory osteolysis, pelvic inflammatory disease, inflammation due to trauma, pharyngitis, dermatitis (including allergic contacts dermatitis), chronic peptic ulcers, periodontitis, and chronic inflammation resulting from chronic viral or bacterial infections.

Some, but not all, types of arthritis are the result of misdirected inflammation. Arthritis is a general term that describes inflammation in the joints. Some types of arthritis associated with inflammation include Rheumatoid arthritis, Psoriatic arthritis and Gouty arthritis. Other painful conditions of the joints and musculoskeletal system that may not be associated with inflammation include osteoarthritis, fibromyalgia, muscular low back pain, and muscular neck pain.

C. Effective Amounts and Controls

SAP can be administered therapeutically to achieve a therapeutic benefit, or prophylactically to achieve a prophylactic benefit, or to achieve both a therapeutic and prophylactic benefit.

An effective amount may refer to the amount of SAP sufficient to reduce or minimize one or more symptoms of an autoimmune response or an inflammatory response or a transplant rejection. An effective amount may also refer to the amount of the SAP that provides a benefit in the management of a symptom of a disease.

In preferred embodiments, SAP are retained at the site of administration (e.g., at the site of inflammation) for periods of time sufficient to yield a beneficial effect. Typically, SAP are administered to the site of undesirable inflammation in an effective amount in order to achieve a clinically significant result. Effective dosages and concentrations are those that provide a benefit, and can be determined according to the desired therapeutic or prophylactic result. For example, the SAP can be effective to reduce or prevent one or more symptoms of an inflammatory disease or disorder. Exemplary symptoms that can be prevented, reduced or otherwise moderated by SAP include, but are not limited to, pain, irritation, inflammation and swelling of the tissue, redness or discoloration of the surrounding tissues, headache, fever, and combinations thereof.

In a particular embodiment, the SAP are administered in an amount effective to reduce or prevent pain and/or swelling due to inflammation at a joint.

In some embodiments, the SAP are administered to a site of inflammation, or a site at risk of inflammation in an amount effective to provide a fluid impermeable barrier at the site of administration. In some embodiments, the barrier structure is effective to prevent the movement of bodily fluids and contaminants through the structure. Therefore, the SAP can reduce or prevent the passage of pro-inflammatory cells and/or signals from one location in the body to another. For example, in some embodiments, the SAP reduce inflammation at a site of infected or damaged tissue by preventing or reducing movement of endogenous pro-inflammatory cells into a site of infection or tissue damage. In other embodiments, the SAP prevent or reduce one or more symptoms of inflammation at a site of infected or damaged tissue by preventing or reducing movement of pro-inflammatory cells or substances away from the site of infection or tissue damage. Reducing inflammation, for example, by providing a physical barrier to the passage of contaminants, pathogens, pro-inflammatory cells, or other agents, can induce reduction or prevention of the physiological responses of tissue damage, induce or enhance healing, reduce or preventing scaring, or combinations of these.

For guidance (e.g., on dosages and concentrations), one can consult texts such as Goodman and Gilman's The Pharmacological Basis of Therapeutics, 10th Ed., and Katzung, Basic and Clinical Pharmacology.

Symptoms of inflammation and immune cell activation can be measured before and after administration of the compositions by methods known in the art. A subject, for example, a human patient, is evaluated on several post-administration days, for example at 1, 7, 14 and 30 days post-administration, using the same evaluation procedure on each day. One or more measurements, for example about five, are obtained for each subject at each time point. The examination can involve observation of criteria such as the presence of cells, flare and fibrin.

All references cited herein are incorporated by reference in their entirety. The present description will be further understood by reference to the following non-limiting examples.

### Example 1: SAP Reduce Immune Cell Action

The anti-inflammatory effect of a SAP was examined using an in vitro assay to assess cytokine release by human THP-1 cells in the presence of SAP. Measurement of THP-1 cell activation in response to antigen was assessed by cytokine release.

Measurement of Effects of SAPs on Control (Resting) T-Cells

Human THP-1 cells in culture media (1×10⁶ cells in 1 ml of Culture medium) were exposed to the self-assembling peptide RADARADARADARADA (“RADA-16”; SEQ ID NO: 1), at a concentration of 1 μg/well, 10 μg/well, or 100 μg/well.

Human THP-1 cells in the absence of RADA-16 were used as a control to assess the baseline level of cytokine production in “resting” non-actived THP-1 cells.

To assess cytokine release upon THP-1 cell activation, lipopolysaccharides (LPS) from Salmonella typhimurium (catalog no. L-7261; Sigma-Aldrich, St. Louis, MO) were added to THP-1 cells, at a concentration of 1 μg/ml.

Measurement of Effects of SAPs on T-Cell Response to Activation

To determine the effects of exposing THP-1 cells to RADA-16 (SEQ ID NO: 1) upon cytokine release, LPS was added to each of the 1 μg/well, 10 μg/well, or 100 μg/well RADA-16 samples, and then added to THP-1 cells.

A potential direct interaction between the lipopolysaccharides (LPS) and the SAP material was examined by gel electrophoresis.

The release of cytokines in response to exposure of THP-1 cells to RADA-16 (SEQ ID NO: 1) or LPS was measured using the PROTEOME PROFILER™ Array kit, commercially available as a test strip kit for detection of cytokines in solution (R&D Systems, Inc., Catalog Number ARY005). The assay was carried out according to the manufacturer's guide lines.

### Results

RADA-16 (SEQ ID NO: 1) did not induce cytokine release beyond baseline levels at any of the concentrations tested, as set forth in Table 4, below.

Electrophoresis revealed no interaction between the lipopolysaccharides and SAP.

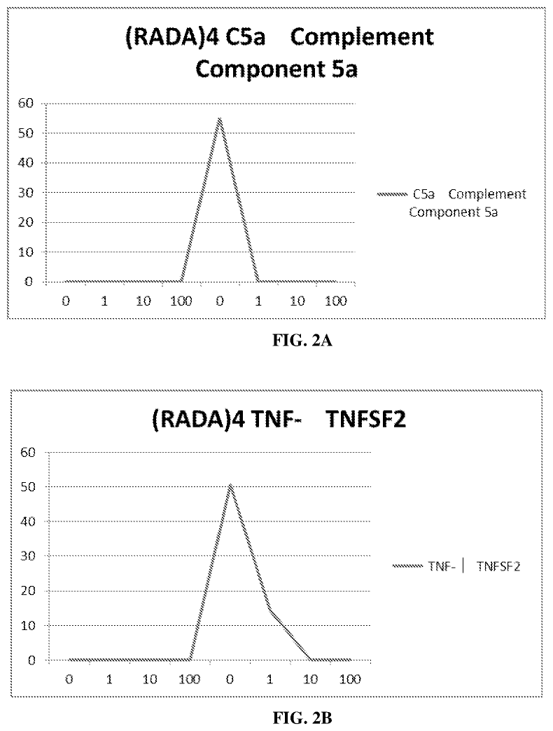

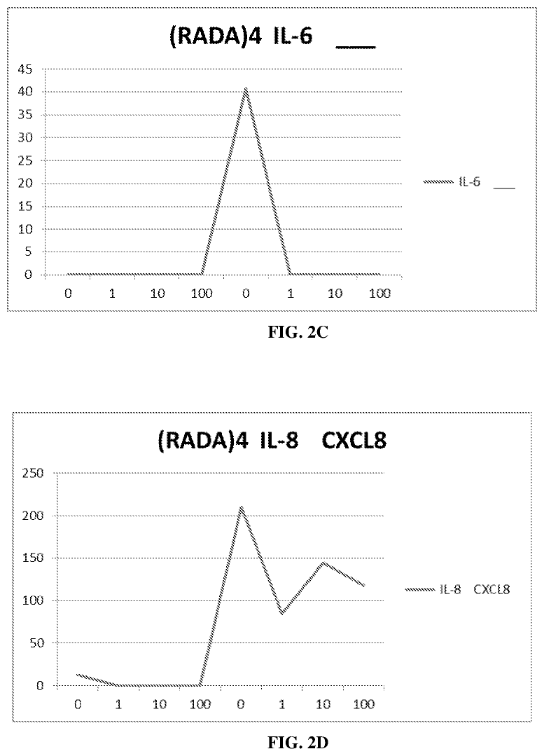

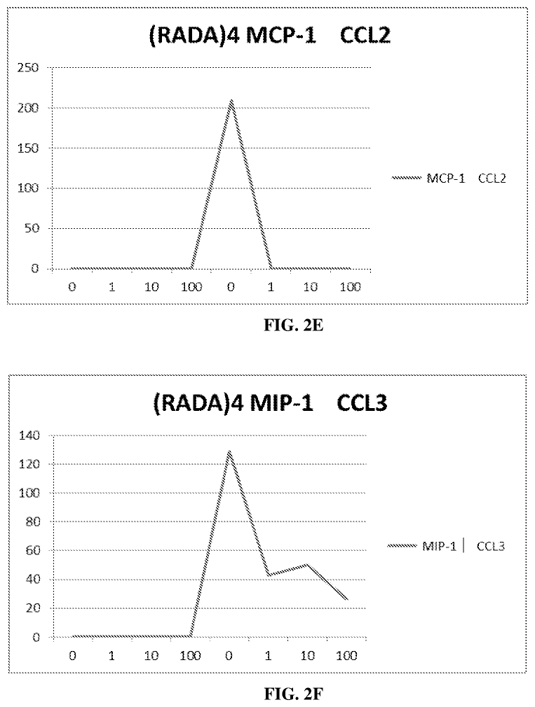

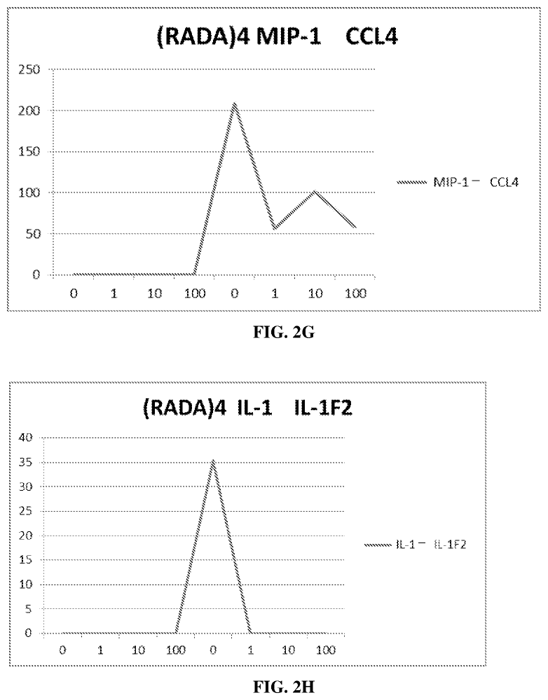

When LPS was added to the RADA-16 (SEQ ID NO: 1) solution and cells were exposed to the LPS/RADA mixture, reduced levels of cytokine release were observed for each of complement component C5a, TNFSF2, IL-6, IL-8/CXCL8, MCP-1/CCL2, MIP-1/CCL3, MIP-1/CCL4, and IL-1/IL1-F2, as depicted in FIGS. 2A-2H.

The presence of RADA-16 (SEQ ID NO: 1) prevented or reduced the release of cytokines by THP-1 cells upon exposure to antigen (LPS). Similar results were observed for other self-assembling sequences.

TABLE 4
Results of cytokine release assay for cells stimulated with 1 μg/well, 10
μg/well, or 100 μg/well RADA-16 (SEQ ID NO: 1), or LPS, and untreated cells
only, as a control (baseline).

| Target/Control | Alternate Nomenclature | Cells Only | 1 mcg | 10 mcg | 100 mcg | LPS only |
|---|---|---|---|---|---|---|
| C5a | Complement Component 5a | 0 | 0 | 0 | 0 | 54.92455 |
| CD40 Ligand | CD154 | 0 | 0 | 0 | 0 | 0 |
| G-CSF | CSF −, CSF-3 | 0 | 0 | 0 | 0 | 0 |
| GM-CSF | CSF \|, CSF-2 | 0 | 0 | 0 | 0 | 0 |
| GRO \| | CXCL1 | 0 | 0 | 0 | 0 | 184.9265 |
| I-309 | CCL1 | 0 | 0 | 0 | 0 | 18.14816 |
| sICAM-1 | CD54 | 0 | 0 | 0 | 0 | 63.70129 |
| IFN-• | Type II IFN | 0 | 0 | 0 | 0 | 0 |
| IL-1 alpha | IL-1F1 | 0 | 0 | 0 | 0 | 0 |
| IL-1 beta | IL-1F2 | 0 | 0 | 0 | 0 | 61.95086 |
| IL-1ra | IL-1F3 | 28.261596 | 22.59339 | 0 | 0 | 206.5893 |
| IL-2 | — | 0 | 0 | 0 | 0 | 0 |
| IL-4 | — | 0 | 0 | 0 | 0 | 0 |
| IL-5 | — | 0 | 0 | 0 | 0 | 0 |
| IL-6 | — | 0 | 0 | 0 | 0 | 40.68697 |
| IL-8 | CXCL8 | 0 | 0 | 0 | 0 | 203.5043 |
| IL-10 | — | 0 | 0 | 0 | 0 | 0 |
| IL-12 p 70 | — | 0 | 0 | 0 | 0 | 0 |
| IL-13 | — | 0 | 0 | 0 | 0 | 0 |
| IL-16 | LCF | 3.665289 | 0 | 0 | 0 | 0 |
| IL-17 | — | 0 | 0 | 0 | 0 | 0 |
| IL-17E | — | 0 | 0 | 0 | 0 | 0 |
| IL-23 | — | 0 | 0 | 0 | 0 | 37.50221 |
| IL-27 | — | 0 | 0 | 0 | 0 | 0 |
| IL-32 | — | 0 | 0 | 0 | 0 | 0 |
| IP-10 | CXCL10 | 0 | 0 | 0 | 0 | 207.8142 |
| I-TAC | CXCL11 | 0 | 0 | 0 | 0 | 0 |
| MCP-1 | CCL2 | 0 | 0 | 0 | 0 | 208.6499 |
| MIP-1 \| | CCL3 | 0 | 0 | 0 | 0 | 172.1709 |
| MIP-1− | CCL4 | 0 | 0 | 0 | 0 | 202.7394 |
| SDF-1 | CXCL12 | 0 | 0 | 0 | 0 | 0 |
| TNF- \| | TNFSF2 | 0 | 0 | 0 | 0 | 73.54276 |
| sTREM-1 | — | 0 | 0 | 0 | 0 | 0 |
| Negative Control | — | 0 | 0 | 0 | 0 | 0 |

### Example 2: SAP Exhibit Anti-Inflammatory Effects in the Liver

### Methods

The anti-inflammatory effects of EARA-16 (SEQ ID NO: 89) and RADA-16 (SEQ ID NO: 1) SAPs were examined using a liver injury model. Levels of inflammation were assayed through immuno-histochemical ED-1 reactivity (ED-1 serves as a marker of macrophage activation).

Pigs or adult Sprague-Dawley rats were anesthetized and their intraperitoneal cavities were opened under aseptic conditions. For each animal, the liver was exposed and then subjected to an 8 mm (pig) or 4 mm (rat) punch biopsy.

The punch sites were then cauterized or treated with acid (for pH control), saline, or RADA-16 (SEQ ID NO: 1) or EARA-16 (SEQ ID NO: 89) solutions. The animals were allowed to survive for up to 14 days. The animals were then sacrificed and their livers were harvested and sectioned. An ED-1 antibody was used to determine the level of inflammation and activation of macrophages in the liver via immunohistochemistry (IHC).

### Results

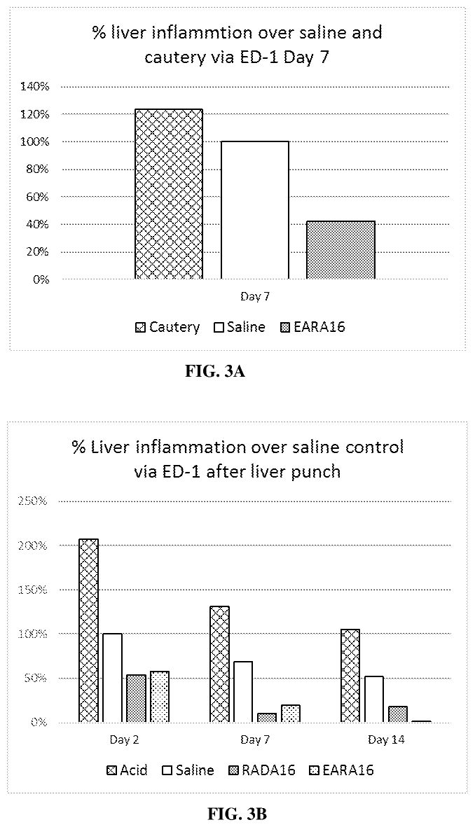

Using the pig liver injury model, application of EARA-16 (SEQ ID NO: 89) markedly reduced the levels of inflammation compared to the saline or cauterization controls observed on day 7 post-treatment (FIG. 3A).

In the rat liver injury model, compared to the saline or acid controls, application of EARA-16 (SEQ ID NO: 89) or RADA-16 (SEQ ID NO: 1) markedly reduced the levels of inflammation at all time-points observed (FIG. 3B).

These data demonstrate that self-assembling peptides such as RADA-16 and EARA-16 reduce and/or prevent inflammation when applied to an organ or tissue (e.g., at a site of injury thereon).

### Example 3: SAP Exhibit Anti-Inflammatory Effects in an Acute Kidney Injury Model

### Methods

The anti-inflammatory effects of EARA-16 (SEQ ID NO: 89) and a RADA-kidney specific peptide conjugate (Ac-(RADA)₃CVSVPQAL-CONH₂; SEQ ID NO: 413; referred to as KS) were examined. Specifically, the effects of EARA-16 and KS on inflammation caused by burn damage were examined using an acute kidney injury model. Levels of inflammation were assayed through immuno-histochemical ED-1 reactivity (ED-1 serves as a marker of macrophage activation).

Adult Sprague-Dawley rats were anesthetized and their intraperitoneal cavities were opened under aseptic conditions. For each animal, the kidney was exposed and then subjected to a stab wound injury.

Cautery was then performed at the injury site to stop bleeding. After cauterization, the injury site was either left untreated and the animals were closed or the site was treated with a 3% solution of EARA-16 (SEQ ID NO: 89) or a 1% solution of KS (SEQ ID NO: 413). The animals were allowed to survive for up to 14 days. The animals were then sacrificed and their kidneys were harvested and sectioned. An ED-1 antibody was used to determine the level of inflammation and activation of macrophages in the kidney via immunohistochemistry (IHC). For each experimental condition, n=3 (max).

Animals receiving no injury, but in which saline was applied to the kidney in a similar location to that of the experimental animals, were used as controls (n=8).

### Results

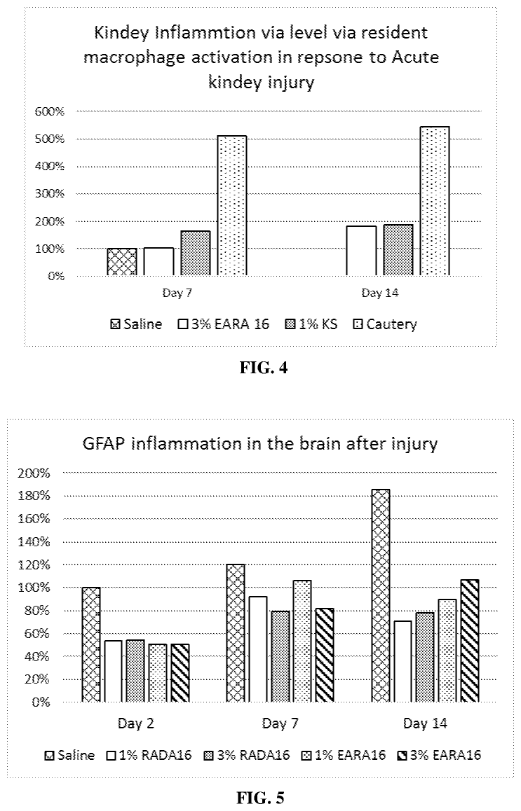

After the animals were sacrificed, their kidneys were examined and IHC was performed to determine the activation level of the kidney resident macrophages. The SAPs effectively reduced inflammation after acute kidney injury (FIG. 4). A reduction in inflammation was observed in the treated cases compared to the cautery only controls (FIG. 4). The cautery only condition showed extensive inflammation in the injury site and surrounding kidney tissue (FIG. 4). At 7-day and 14-day survival, there was a 68% and 66% reduction respectively in the KS treated group compared to the cautery only condition. At 7-day and 14-day survival, the EARA-16 (SEQ ID NO: 89) treated group showed an 80% and 66% reduction in inflammation respectively compared to the cautery only condition.

This data demonstrates that SAP, including SAP conjugated to a tissue-specific peptide, can be used effectively to reduce inflammation after injury such as a burn or surgery.

### Example 4: SAP Exhibit Anti-Inflammatory Effects in an Olfactory Bulb Injury Model

### Methods

The anti-inflammatory effects of EARA-16 (SEQ ID NO: 89) and RADA-16 (SEQ ID NO: 1) were examined using an olfactory bulb injury model. Levels of inflammation were assayed through immunohistochemical staining for glial fibrillary acidic protein (GFAP) as an indicator of reactive astrocytes.

In brief, the olfactory bulbs of adult Sprague-Dawley rats were unilaterally transected and the cuts were then treated with either saline as a control, or one of RADA-16 (SEQ ID NO: 1) or EARA-16 (SEQ ID NO: 89) solutions (each used at 1% and 3%). The SAP materials were injected into the cuts post transection at the time of surgery. After 2, 7 or 14 days, the animals were sacrificed.

A more detailed protocol is as follows: adult Sprague-Dawley rats were anesthetized with a mixture of ketamine (80 mg/kg, i. p.) and xylazine (8 mg/kg, i. p.). The head was shaved and a thorough disinfection of the head surface was carried out with iodine 5% solution. The bilateral olfactory bulbs were exposed by drilling two burr holes (2 mm diameter, 8 mm anterior to the bregma) on either side at a distance of 2 mm from the midline of the frontal bone in the region overlying the olfactory bulbs. A cataract knife with a blunted end and one sharpened edge was used for surgery. The cut was performed on the middle of olfactory bulb. The blunted edge of the knife was inserted along the middle line of the olfactory bulb to prevent damage of the sagittal sinus, until the tip of the knife lightly touched the bottom of the brain without penetration of the dura. The knife was then moved laterally to transect the bulb until it reached the lateral edge of the hole. The knife was then rotated and pulled out to have a complete cut of the bulb at lateral side. This procedure was modified from the method described in several previous studies attempting to isolate the olfactory bulb from other parts of the brain (Ahrens & Freeman 2001; Allison 1953; Chaput 1983; Freeman 1968; Koliatsos et al 2004; Munirathinam et al 1997).

After performing the cut, one of the solutions was used to treat the injury site: either 10 μl saline (control group) or the EARA-16 (SEQ ID NO: 89) or RADA-16 (SEQ ID NO: 1) SAP (at a concentration of 1% and 3%) was injected evenly into the wound site using a self-constructed hydraulic pressure injection system without micro syringe. This technique was modified from similar methods as described by Bullier and others (Bullier et al 1980; Fish & Rhoades 1981; Kratskin et al 1997; Meyers & Snow 1981; Price et al 1977; Shipley 1982). Care was taken to control the speed of the injection of the saline or the SAP. After the injection, the wound was closed and the animals were placed in a warm place for recovery before they were brought back to their home cages.

At 2, 7 and 14 days after the incision, the animals were sacrificed with an overdose of pentobarbital (160 mg/kg body weight) and perfused transcardially with 40.01M phosphate buffer solution (PBS) followed by 4% paraformaldehyde in 0.1 M phosphate buffer (pH 7.4).PBS. The brains were excised and post-fixed in 4% paraformaldehyde overnight at a temperature of 4° C. The brains were then cryoprotected with a 30% sucrose solution at 4° C. The brains were embedded with Optimal Cutting Temperature compound (Tissue-Tek® O.C.T., Ted Pella, Inc. Redding, CA). The brains including the olfactory bulbs were sectioned and placed on subbed slides. Sagittal sections were cut with a thickness of 15 μm using a freezing cryostat (Leica CM1900, Leica Micorsystems, 35578 Wetzlar, Germany). Fifteen sections were obtained. The sections were mounted on superfrost plus pre-coated slides (Thermo Scientific; Thermo Fisher Scientific, Waltham, MA 02454 USA) and were processed for immunohistochemistry.

Briefly, each step was preceded with 3 washes in 0.01M PBS for 5 min each. Pre-blocking was performed with immersion in a blocking solution containing 0.3% triton, 2.5% bovine serum albumin (BSA; Proliant Co, Ankeny, IA, USA) and 10% goat serum at room temperature for 1 hour. The sections were then incubated in a primary antibody diluted solution overnight at 4° C., containing rabbit anti-IBA1 (Ionized calcium Binding Adaptor molecule 1; 1:500; Wako Pure Chemical Industries, Ltd. Osaka, Japan) and mouse anti rat CD 68 (ED1) (1:1000; AbD Serotech, D-40470 Dusseldorf, Germany). Serial sections were incubated with mouse anti-GFAP (sigma, 1:1000). The sections were then washed in 0.01M phosphate buffered saline (10 minutes, 3 times), and incubated with secondary antibody diluted solution at room temperature for 2 hours (Goat-anti-rabbit 568, Goat-anti-mouse Alexa 488, (Invitrogen, Carlsbad, California, 1:400)). The slides were then washed in 0.01M phosphate buffered saline (10 minutes, 3 times) cover slipped with fluorescein mounting medium with DAPI (4′,6-diamidin-2′-phenylindol-dihydrochlorid; Dako Ltd, Glostrup, Denmark). All images were taken under a 20× objective with a Carl Zeiss Flour microscope (Carl Zeiss Inc. Oberkochen, Germany) fitted with a spot camera. All illumination levels were fixed during image acquisition.

### Results

Across all time points, it was observed that treatment with the SAP (at both concentrations evaluated) reduced the relative inflammation when compared to the saline controls (FIG. 5). In several cases (1% and 3% RADA-16 (SEQ ID NO: 1) and 1% EARA-16 (SEQ ID NO: 89)), there was an increased activation of reactive astrocytes on day 7 which subsequently reduced by day 14 (FIG. 5). Inflammation levels in the saline controls continued to increase over the 14 days. It also appeared that the 3% EARA-16 (SEQ ID NO: 89) group exhibited increasing inflammation levels, but at a much slower rate than the saline controls. N=7 for each case.

1. (canceled)

2. The method of claim 1, wherein the self-assembling peptides or self-assembling peptidomimetics consist of a sequence of amino acid residues conforming to one or more of Formulas I-IV:
((Xaa^{neu}−Xaa⁺)ₓ(Xaa^{neu}−Xaa⁻)_{y})ₙ (I)
((Xaa^{neu}−Xaa⁻)ₓ(Xaa^{neu}−Xaa⁺)_{y})ₙ (II)
((Xaa⁺−Xaa^{neu})ₓ(Xaa⁻−Xaa^{neu})_{y})ₙ (III)
((Xaa⁻−Xaa^{neu})ₓ(Xaa⁺−Xaa^{neu})_{y})ₙ (IV)
wherein Xaa^{neu} represents alanine; Xaa⁺ represents arginine or lysine; Xaa⁻ represents aspartic acid or glutamic acid; x and y are 1; and n is an integer having a value of 2 to 4.

3. The method of claim 12, wherein between about 70% and 100% of all of the self-assembling peptides or self-assembling peptidomimetics are of the same size and have the same amino acid sequence.

4. The method of claim 12, wherein the composition further comprises a pharmaceutically acceptable excipient for administration into or onto a site of inflammation.

5. (canceled)

6. The method of claim 12, further comprising one or more therapeutic agents, prophylactic agents, antimicrobial agents, diagnostic agents, and combinations thereof for treatment and/or alleviation of one or more symptoms of a disorder associated with the inflammation.

7. The method of claim 12, wherein the composition is in a form selected from the group consisting of powders, liquids, gels, a coating on a medical device, emulsions, eye patches, gauzes and bandages.

8. The method of claim 12, wherein the composition is dried.

9. The method of claim 12, wherein the concentration of self-assembling peptides or self-assembling peptidomimetics in the composition is between about 1.0% w/v and about 6% w/v, inclusive.

10. The method of claim 12, wherein the concentration of ions in the composition is less than 5 mM.

11. The method of claim 12, wherein the composition is formulated for administration into a joint in the form of an intra-articular injection.

12. A method for reducing or preventing one or more of the symptoms of inflammation in a subject in need thereof, the method comprising
administering to the subject at a site of inflammation or at risk of inflammation a composition comprising self-assembling peptides or self-assembling peptidomimetics in an amount effective to reduce or prevent one or more symptoms selected from the group consisting of pain, irritation, swelling, redness or other discoloration, loss of sensation, reduced mobility, fever, headache, itching, discharge of pus, headache, chills, muscle stiffness, immobility of a joint, loss of function of an organ, stimulation of nerve endings by bradykinin, increased blood flow, malaise, and physiological responses associated with production of histamine and/or heparin,
wherein the self-assembling peptides or self-assembling peptidomimetics consist of a sequence of amino acid residues conforming to one or more of Formulas I-XII:
((Xaa^{neu}−Xaa⁺)ₓ(Xaa^{neu}−Xaa⁻)_{y})ₙ (I)
((Xaa^{neu}−Xaa⁻)ₓ(Xaa^{neu}−Xaa⁺)_{y})ₙ (II)
((Xaa⁺−Xaa^{neu})ₓ(Xaa⁻−Xaa^{neu})_{y})ₙ (III)
((Xaa⁻−Xaa^{neu})ₓ(Xaa⁺−Xaa^{neu})_{y})ₙ (IV)
Xaa^{neu}((Xaa^{neu}−Xaa⁺)ₓ(Xaa^{neu}−Xaa⁻)_{y})ₙ (V)
Xaa^{neu}((Xaa^{neu}−Xaa⁻)ₓ(Xaa^{neu}−Xaa⁺)_{y})ₙ (VI)
((Xaa⁺−Xaa^{neu})ₓ(Xaa⁻−Xaa^{neu})_{y})ₙXaa^{neu} (VII)
((Xaa⁻−Xaa^{neu})ₓ(Xaa⁺−Xaa^{neu})_{y})ₙXaa^{neu} (VIII)
((Xaa^{neu}−Xaa⁺)ₓ(Xaa^{neu}−Xaa⁻)_{y})ₙXaa^{neu} (IX)
((Xaa^{neu}−Xaa⁻)ₓ(Xaa^{neu}−Xaa⁺)_{y})ₙXaa^{neu} (X)
Xaa^{neu}((Xaa⁺−Xaa^{neu})ₓ(Xaa⁻−Xaa^{neu})_{y})ₙ (XI)
Xaa^{neu}((Xaa⁻−Xaa^{neu})ₓ(Xaa⁺−Xaa^{neu})_{y})ₙ (XII)
wherein Xaa^{neu} represents an amino acid residue having a neutral charge; Xaa⁺ represents an amino acid residue having a positive charge; Xaa⁻ represents an amino acid residue having a negative charge; x and y are integers having a value of 1, 2, 3, or 4, independently; and n is an integer having a value of 1-5; and
wherein the self-assembling peptides or self-assembling peptidomimetics form a barrier to the movement of bodily substances at the site of administration.

13. The method of claim 12, wherein the subject has or is at risk of developing a disease or disorder selected from the group consisting of asthma, encephalitis, inflammatory bowel disease, chronic obstructive pulmonary disease, allergic disorders, septic shock, pulmonary fibrosis, undifferentiated spondyloarthropathy, undifferentiated arthropathy, arthritis, inflammatory osteolysis, and chronic inflammation resulting from chronic viral or bacterial infections.

14. The method of claim 13, wherein the subject suffers from arthritis.

15. The method of claim 12, wherein the self-assembling peptides or peptidomimetics are self-assembled immediately prior to, at the time of, or after administration of the composition.

16. (canceled)

17. The method of claim 15, wherein the peptides are self-assembled by contacting the self-assembling peptides or peptidomimetics with a solution of cations.

18. The method of claim 12, comprising multiple administrations, separated in time by one or more minutes, hours or days, wherein the multiple administrations are carried out for a period of up to one week, up to one month, or up to one year.

19. The method of claim 18, wherein each administration comprises administering a different formulation of self-assembling peptides or self-assembling peptidomimetics to the same site.

20. The method of claim 12, wherein the composition is administered at a site of inflammation or at risk of inflammation selected from skin, mucosa, liver, lung, intestine, brain, stomach, muscle, bone, spleen, kidney, bladder, heart, joints, tissues of the genitourinary system, or tissues of the central nervous system.

21. The method of claim 12, wherein the composition is partially or completely biodegradable.

22. The method of claim 12, wherein the composition is packaged with a desiccant and/or a pH-adjusting agent.

23. The method of claim 9, wherein the concentration of self-assembling peptides or self-assembling peptidomimetics in the composition is between about 1.0% w/v and about 4% w/v, inclusive.
